(19) United States
(12) Patent Application Publication
(10) Pub. No.: US 2015/0313892 A1
(43) Pub. Date: Nov. 5, 2015

(54) PHARMACEUTICAL COMPOSITIONS FOR TREATING PAIN ASSOCIATED WITH DYSMENORRHEA

(71) Applicant: Trinity Laboratories, Inc., San Antonio, TX (US)

(72) Inventors: Chandra U. Singh, San Antonio, TX (US); Jagaveerabhadra Rao Nulu, Austin, TX (US); David Woody, San Antonio, TX (US)

(21) Appl. No.: 14/617,730

(22) Filed: Feb. 9, 2015

Related U.S. Application Data
(60) Provisional application No. 61/937,055, filed on Feb. 7, 2014.

(51) Int. Cl. A61K 31/485; A61K 9/48; A61K 31/197; A61K 33/06; A61K 31/135; A61K 31/195

(52) U.S. Cl. CPC A61K 31/485; A61K 31/135; A61K 31/195; A61K 31/197; A61K 33/06; A61K 9/4808

(57) ABSTRACT

Pain associated with primary and secondary dysmenorrhea is relieved in a human suffering there from by administering to the human a pain relieving amount of a synergistically acting sub-therapeutic combination of a nontoxic N-methyl-D-aspartate receptor antagonist such as dextromethorphan, magnesium, dextrorphan, ketamine or pharmaceutically acceptable salt thereof, tramadol or its analog such as recemic tramadol or an analogously acting molecular entity or pharmaceutically acceptable salt thereof, and an anticonvulsant and/or a tricyclic anti-depressant or pharmaceutically acceptable salt thereof, and optionally in sustained release dosage form.

### CROSS REFERENCE TO RELATED APPLICATIONS

This application claims the benefit of U.S. Provisional Appl. No. 61/937,055, filed Feb. 7, 2014, which is incorporated by reference herein in its entirety.

### BACKGROUND OF THE INVENTION

Dysmenorrhea (or dysmenorrhoea) is a gynecological medical condition of pain during menstruation that interferes with daily activities, as defined by, The American Congress of Obstetricians and Gynecologists (ACOG). Still, dysmenorrhea is often defined simply as menstrual pain, or at least menstrual pain that is excessive. Menstrual pain is often used synonymously with menstrual cramps, but the latter may also refer to menstrual uterine contractions, which are generally of higher strength, duration and frequency than in the rest of the menstrual cycle. Dysmenorrhea can feature different kinds of pain, including sharp, throbbing, dull, nauseating, burning, or shooting pain. Dysmenorrhea may precede menstruation by several days or may accompany it, and it usually subsides as menstruation tapers off (Harel Z. Dysmenorrhea in Adolescents and Young Adults: Etiology and Management. J Pediatr Adolesc Gynecol. 2006; 19(6):363-371).

Secondary dysmenorrhea is diagnosed when symptoms are attributable to an underlying disease, disorder, or structural abnormality either within or outside the uterus. Primary dysmenorrhea is diagnosed when none of these are detected. Dysmenorrhea can be classified as either primary or secondary based on the absence or presence of an underlying cause. The most common cause of secondary dysmenorrhea is endometriosis (Cramer D W, Missmer S A. The epidemiology of endometriosis. Ann N Y Acad Sci. 2002; 955:11-22). Other causes include leiomyoma, adenomyosis, ovarian cysts, and pelvic congestions. The presence of a copper IUD can also cause dysmenorrhea.

Menstrual pain was reported by 84.1% of women, with 43.1% reporting that pain occurred during every period, and 41% reporting that pain occurred during some periods (Giovanni G, et al. Prevalence of menstrual pain in young women: what is dysmenorrhea?. J Pain Res. 2012; 5:169-174). Reports of dysmenorrhea are greatest among individuals in their late teens and 20s, with reports usually declining with age. The prevalence in adolescent females has been reported to be 67.2% by one study (Sharma P, et al. Problems related to menstruation amongst adolescent girls. Indian J Pediatr 2008; 75 (2): 125-9). It has been stated that there is no significant difference in prevalence or incidence between races.

The uterus frequently contracts throughout the entire menstrual cycle, and these contractions have been termed endometrial waves or contractile waves (Aguilar H N, et al. Physiological pathways and molecular mechanisms regulating uterine contractility. Human Reproduction Update 2010; 16 (6): 725-744). These appear to involve only the sub-endometrial layer of the myometrium. In the early follicular phase, these contractions occur once or twice per minute and last 10-15 seconds with a low amplitude of usually 30 mmHg. The frequency increases to 3-4 per minute towards ovulation. During the luteal phase, the frequency and amplitude decrease, possibly to facilitate any implantation. If implantation does not occur, the frequency remains low, but the amplitude increases dramatically to between 50 and 200 mmHg producing labor-like contractions at the time of menstruation. A shift in the myosin expression of the uterine smooth muscle has been hypothesized to avail for changes in the directions of uterine contractions that are seen during the menstrual cycle.

The main symptom of dysmenorrhea is pain concentrated in the lower abdomen, in the umbilical region or the suprapubic region of the abdomen. It is also commonly felt in the right or left abdomen. It may radiate to the thighs and lower back. Symptoms often co-occurring with menstrual pain include nausea and vomiting, diarrhea or constipation, headache, dizziness, disorientation, hypersensitivity to sound, light, smell and touch, fainting, and fatigue. Symptoms of dysmenorrhea often begin immediately following ovulation and can last until the end of menstruation. This is because dysmenorrhea is often associated with changes in hormonal levels in the body that occur with ovulation. The use of certain types of birth control pills can prevent the symptoms of dysmenorrhea, because the birth control pills stop ovulation from occurring. During a woman's menstrual cycle, the endometrium thickens in preparation for potential pregnancy. After ovulation, if the ovum is not fertilized and there is no pregnancy, the built-up uterine tissue is not needed and thus shed.

Prostaglandins are released during menstruation, due to the destruction of the endometrial cells, and the resultant release of their contents (Lethaby A, et al. (2007). Lethaby, Anne. ed. “Nonsteroidal anti-inflammatory drugs for heavy menstrual bleeding”. Cochrane Database Syst Rev (4): CD000400). Release of prostaglandins and other inflammatory mediators in the uterus cause the uterus to contract. These substances are thought to be a major factor in primary dysmenorrhea (Wright, et al. The Washington Manual Obstetrics and Gynecology Survival Guide. Lippincott Williams and Wilkins, 2003). When the uterine muscles contract, they constrict the blood supply to the tissue of the endometrium, which, in turn, breaks down and dies. These uterine contractions continue as they squeeze the old, dead endometrial tissue through the cervix and out of the body through the vagina. These contractions, and the resulting temporary oxygen deprivation to nearby tissues, are responsible for the pain or “cramps” experienced during menstruation.

Compared with other women, females with primary dysmenorrhea have increased activity of the uterine muscle with increased contractility and increased frequency of contractions (Rosenwaks Z, Seegar-Jones G. Menstrual pain: its origin and pathogenesis. J Reprod Me, 1980; 25 (4 Supply: 207-12). In one research study using MRI, visible features of the uterus were compared in dysmenorrheic and eumenorrheic (normal) participants. The study concluded that in dysmenorrheic patients, visible features on cycle days 1-3 correlated with the degree of pain, and differed significantly from the control group (Kataoka M, et al. Dysmenorrhea: evaluation with cine-mode-display MR imaging—initial experience”. Radiology 2005; 235 (1): 124-31).

Non-steroidal anti-inflammatory drugs (NSAIDs) are effective in relieving the pain of primary dysmenorrhea (Marjoribanks J, et al., in Marjoribanks, Jane. ed. “Nonsteroidal anti-inflammatory drugs for dysmenorrhoea”. Cochrane database of systematic reviews (Online) (1): CD001751). They can have side effects of nausea, dyspepsia, peptic ulcer, and diarrhea. Patients who are unable to take the more common NSAIDs, may be prescribed a COX-2 inhibitor (Chantler I, et al. The effect of three cyclo-oxygenase inhibitors on intensity of primary dysmenorrheic pain. Clin J Pain 2008; 24 (1): 39-44). Besides these drugs anti-spasmodic's like drotravine is used that relax the muscles and helps to reduce the pain.

Although use of hormonal contraception can improve or relieve symptoms of primary dysmenorrhea, a 2001 systematic review found that no conclusions can be made about the efficacy of commonly used modern lower dose combined oral contraceptive pills for primary dysmenorrhea (Proctor M L, et al. (2001). Wong, Chooi L. ed. “Combined oral contraceptive pill (OCP) as treatment for primary dysmenorrhoea”. Cochrane Database Syst Rev (4): CD002120). A review indicated the effectiveness of use of transdermal nitroglycerin (Morgan P J, et al. Nitroglycerin as a uterine relaxant: a systematic review. J Obstet Gynaecol Can 2008; 24 (5): 403-9).

A number of alternative therapies have been studied in the treatment of dysmenorrhea. The effectiveness of acupressure, behavioral interventions, thiamine, vitamin E, topical heat, and transcutaneous electrical nerve stimulation is likely while the effects of acupuncture, fish oil, magnets and vitamin B12 is unknown. A 2008 systematic review found promising evidence for Chinese herbal medicine for primary dysmenorrhea, but that the evidence was limited by its poor methodological quality (Zhu X, et al. (2008). Zhu, Xiaoshu. ed. “Chinese herbal medicine for primary dysmenorrhoea”. Cochrane Database Syst Rev (2): CD005288).

Behavioral therapies assume that the physiological process underlying dysmenorrhea is influenced by environmental and psychological factors, and that dysmenorrhea can be effectively treated by physical and cognitive procedures that focus on coping strategies for the symptoms rather than on changes to the underlying processes. A 2007 systematic review found some scientific evidence that behavioral interventions may be effective, but that the results should be viewed with caution due to poor quality of the data (Proctor M L, et al. (2007). Proctor, Michelle. ed. Behavioural interventions for primary and secondary dysmenorrhoea. Cochrane Database Syst Rev (3): CD002248). Acupuncture and acupressure are used to treat dysmenorrhea. A review cited four studies, two of which were patient-blind, indicating that acupuncture and acupressure were effective (White A. A review of controlled trials of acupuncture for women's reproductive health care. J Fam Plann Reprod Health Care 2003; 29 (4): 233-6).

Pain is generally defined as an unpleasant sensory and emotional experience associated with actual or potential tissue damage or described in terms of such damage. The noxious stimulation causes a release of chemical mediators from the damaged cells including: prostaglandin, bradykinin, serotonin, substance P, potassium and histamine. These chemical mediators activate and/or sensitise the nociceptors to the noxious stimuli. In order for a pain impulse to be generated, an exchange of sodium and potassium ions (de-polarisation and re-polarisation) occurs at the cell membranes. This results in an action potential and generation of a pain impulse. In the case of neuropathic pain, the pain impulse is generated by the injured neurons through misfiring.

The transmission process of pain occurs in three stages (Dermot J K, et al. Preemptive analgesia I. physiological pathways and pharmacological modalities, 2001; 48:1000-1010). The pain impulse is transmitted: from the site of transduction along the nociceptor fibers to the dorsal horn in the spinal cord; from the spinal cord to the brain stem; through connections between the thalamus, cortex and higher levels of the brain. The C fibre and Aδ fibers terminate in the dorsal horn of the spinal cord. There is a synaptic cleft between the terminal ends of the C fiber and Aδ fibers and the nociceptive dorsal horn neurons (NDHN). In order for the pain impulses to be transmitted across the synaptic cleft to the NDHN, excitatory neurotransmitters are released, which bind to specific receptors in the NDHN. These neurotransmitters are: adenosine triphosphate, glutamate, calcitonin gene-related peptide, bradykinin, nitrous oxide and substance P. The pain impulse is then transmitted from the spinal cord to the brain stem and thalamus via two main nociceptive ascending pathways. These are the spinothalamic pathway and the spinoparabrachial pathway. The brain does not have a discrete pain centre, so when impulses arrive in the thalamus they are directed to multiple areas in the brain where they are processed.

The modulation of pain involves changing or inhibiting transmission of pain impulses in the spinal cord. The multiple, complex pathways involved in the modulation of pain are referred to as the descending modulatory pain pathways (DMPP) and these can lead to either an increase in the transmission of pain impulses (excitatory) or a decrease in transmission (inhibition).

Descending inhibition involves the release of inhibitory neurotransmitters that block or partially block the transmission of pain impulses, and therefore produce analgesia. Inhibitory neurotransmitters involved with the modulation of pain include: endogenous opioids (enkephalins and endorphins), serotonin (5-HT), norepinephirine (noradrenalin), gamma-aminobutyric acid (GABA), neurotensin, acetylcholine and oxytocin. Endogenous opioids are found throughout the central nervous system (CNS) and prevent the release of some excitatory neurotransmitters, for example, substance P, therefore, inhibiting the transmission of pain impulses.

The modern concept of pain treatment emphasizes the significance of prophylactic prevention of pain, as pain is more easily prevented than it is relieved. Pain is generally controlled by the administration of short acting analgesic agents, steroids and non-steroidal anti-inflammatory drugs. Analgesic agents include opiates, agonistic-antagonistic agents, and anti-inflammatory agents.

Opiates, a class of centrally acting compounds, are the most frequently used agents for pain control. Opiates are narcotic agonistic analgesics and are drugs derived from opium, such as morphine, codeine, and many synthetic congeners of morphine, with morphine and hydrocodone preparations being the most widely used opiates. Opiates are natural and synthetic drugs with morphine-like actions. Opiates are narcotic agonistic analgesics which produce drug dependence of the morphine type and are subject to control under Federal narcotics law and the laws of most other nations and international organizations because of their addicting properties and the subsequent destructive toll exacted on the abusers and those with any connection to them. The term “opiates” also includes opiate antagonists that are essentially devoid of agonist activity at any opiate receptor, partial agonists, and opiates with mixed actions, that is they are mixed function agonist-antagonists, which are agonists at some receptors and antagonists at other receptors.

The chemical classes of opiates with morphine like activity are the purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives. The principal phenanthrenes are morphine, codeine, and thebaine. The principal benzoisoquinolines are papaverine, a smooth muscle relaxant, and noscapine. Semi-synthetic derivatives of morphine include diacetylmorphine (heroin), hydromorphone, oxymorphone, hydrocodone, apomorphine, etorpine, and oxycodone. Phenylpiperidine derivatives include meperidine and its congeners diphenoxylate and loperamide, alphaprodine, anileridine hydrochloride or phosphate, and piminodine mesylate. The currently used morphinan derivative is levorphanol. The diphenyl-heptane derivatives include methadone and its congeners, and propoxyphene. Propionanilide derivatives include fentanyl citrate and its congeners sufentanil citrate and alfentanil hydrochloride. These opiate analgesics are discussed in detail in Goodman and Gilman's The Pharmacological Basis of Therapeutics, Chapter 21, Opiate Analgesics and Antagonists, pp. 485-521 (8^{th} ed. 1990), which is incorporated herein by reference.

The potency of all opiates is roughly comparable and can be effective against the most severe pain with appropriate dosing at intervals. In addition to the μ-opiate receptor agonists such as morphine, other classes of analgesic agents that are commonly used include agonistic-antagonistic analgesic agents, non-steroidal anti-inflammatory drugs (NSAIDS), steroids, cyclooxygenase inhibitors, anti-depressants, minerals such as magnesium, tryptan drugs for migraines, ergotamine and related compounds for migrainous headache and dissociative psychoactive drugs. Agonistic-antagonistic analgesic agents are effective for the alleviation of moderate to severe pain, but due to their antagonistic properties, their analgesic efficacy does not increase by increasing the dosage above a certain level.

However, all opiates have a wide variety of side effects that can decrease their clinical utility in certain situations. The side effects associated with the use of opiates include respiratory depression, reduced cough reflex, bronchial spasms, nausea, vomiting, release of histamine, peripheral vasodilation, orthostatic hypotension, alteration of vagal nerve activity of the heart, hyperexcitability of smooth muscles (sphincters), reduction of peristaltic motility in the gastrointestinal tract and urinary retention. Opiates also stimulate the release of adrenaline, anti-diuretic hormone, cause changes in the regulation of body temperature and sleep pattern, and are liable to promote the development of tolerance and addiction.

Furthermore, higher doses of agonistic-antagonistic analgesic agents are often associated with unpleasant sympathomimetic side effects such as tachycardia, increase in blood pressure, seizure and psychotomimetic effects such as drug induced psychosis, hyper-aggressive behavior and agitation. However, the risk of respiratory depression also decreases proportionately with the diminished analgesic activity of the higher doses. Agonistic-antagonistic analgesic agents with pharmacological activity similar to the morphine like opiates include pentazocine, nalbuphine, butorphanol, nalorphine, buprenorphine (a partial agonist), meptazinol, dezocine, and cyclazocine.

The NSAIDs include the salicylates such as salicylamide and acetylsalicylic acid (aspirin). Non-aspirin NSAIDs include para-aminophenol derivatives such as phenacetin, the pyrazole derivatives such as antipyrine, aminopyrine, dypyrone, nefenamic acid, indomethacin, methimazole, paracetamol, diclophenac sodium/potassium, ibuprofen, naproxen, and ketorolac tromethamine, all of which can be combined with opiates or used alone to alleviate milder pain. The mechanism of action of NSAIDs is by direct action at the site of tissue injury. NSAIDs peripherally inhibit cyclooxygenases (COX), the enzymes responsible for providing an activated substrate molecules for the synthesis of prostaglandins, which are a group of short-acting mediators of inflammation. The maximal analgesic effect of a standard 325 mg dose of aspirin or of NSAIDs is adjusted to provide the level of pain relief comparable to that achieved by the administration of five milligrams of morphine administered intramuscularly. The analgesic acetaminophen is often categorized as a NSAID even though the compound does not exhibit significant anti-inflammatory activity. Unless otherwise indicated, acetaminophen will be referred to herein as a NSAID.

Another difficulty that has recently been gaining increasing attention is the negative side effects of non-steroidal anti-inflammatory agents. Side effects of NSAIDs include gastrointestinal irritation, clotting difficulty and secondary anemia, bronchospastic effects in asthmatic mammalian patients, and tinnitus. The overuse of NSAIDS is in fact largely due to the inappropriate under-treatment of pain in individuals who for whatever reason do not use more effective drugs that operate on other parts of the pain pathway.

The analgesic agents are all used in similar ways to treat pain in humans. However, humans will develop tolerance to the analgesic effect and develop psychological and physical dependencies on these agents, especially the opiates, thereby reducing the effectiveness of the pain treatment and exacerbating the suffering of the patient. The long term administration of narcotic analgesics to patients suffering from various types of chronic pain such as causalgia, hyperesthesia, sympathetic dystrophy, phantom limb syndrome, denervation, etc., is subject to a number of serious drawbacks including the development of opiate tolerance and/or dependence, severe constipation, and so forth.

Physical dependence or drug addiction to narcotic drugs has been traditionally treated by drug withdrawal through withholding the opiate from the drug dependent individual, gradually decreasing the amount of opiate taken by the individual, administering an opiate antagonistic drug, or substituting another drug, such as methadone, buprenorphine, or methadyl acetate for the opiate to ameliorate the physical need for the opiate. In addition the psychology of the person is treated through therapeutic interventions such as individual and group therapies. When an opiate is discontinued withdrawal symptoms appear. The character and severity of the withdrawal symptoms are dependent upon such factors as the particular opiate being withdrawn, the daily dose of the opiate, the duration of use of the opiate and the health of the drug dependent individual. The physical and psychological pain associated with withdrawal symptoms can be quite severe.

An alternative approach to pain treatment employing the analgesic agents described above was tried in which an aromatic amino acid, tryptophan, was administered to persons undergoing third molar surgery to alleviate the pain and reduce or eliminate the consumption of other analgesics. The rationale was that serotonin, a neurotransmitter and a component of the serotonergic pain suppression pathway, is synthesized from tryptophan after the tryptophan is transported across the blood-brain barrier. Tryptophan is a precursor of serotonin and it was assumed that it would have analgesic effects. It was found however that tryptophan had no effect on post-operative pain or on the consumption of other analgesics (Ekblom, A, et al. Tryptophan supplementation does not affect post-operative pain intensity or consumption of analgesics. Pain 1991; 44:249-254).

Other treatments include the use of antidepressants, specifically, the tricyclic antidepressants (TCA's), such as amytriptiline. These relieve pain by altering levels of serotonin in the body. The antineuralgic properties of TCA's were shown to be independent from their antidepressant properties. TCA's are associated with a number of adverse side effects such as sedation, orthostatic hypotension, dry mouth, urinary retention, constipation, and weight gain. These side effects are more pronounced in the elderly. TCA's should be used with caution in the elderly, patients with heart disease, narrow angle glaucoma, and prostatism. Another class of antidepressants, the selective serotonin reuptake inhibitors (SSRI's), may also be used. In general, the SSRI's have not been found to be as effective as the TCA's for the treatment of neuroptahic pain, but are better tolerated. The side effects of the SSRI's include sweating, stomach upset, somnolence, dizziness, decreased libido, and ejaculatory disturbances.

Changes in serotonin transport function and in neuroreceptor loading that occur over the course of antidepressant use create a dependence on the drug that takes some time to be eliminated even when the drug is no longer needed to stabilize depression. Adverse effects that can arise from reducing the drug dose have been given a name: SSRI Withdrawal Syndrome or SSRI Discontinuation Syndrome (Bull S A, et al., Discontinuing or switching selective serotonin-reuptake inhibitors, Annals of Pharmacotherapy 2002; 36(4): 578-584). To avoid this syndrome, very gradual withdrawal—as little as 5% dosage decline per week has been recommended; rarely are the drugs withdrawn at a rate of more than 20% per week. Unfortunately, many patients are hesitant to spend this much time withdrawing from the drug, and many physicians do not recommend such gradual dosage decline, believing that the majority of the patients will do well with relatively rapid withdrawal, so SSRI Withdrawal Syndrome can readily occur; some patients may experience the symptoms even with very gradual tapering of dosage.

Dextromethorphan (frequently abbreviated as DM) is the common name for (+)-3-methoxy-N-methylmorphinan (FIG. 1). It widely used as a cough syrup, and is described in references such as Rodd 1960 (full citations to articles are provided below) and Goodman and Gilman's Pharmacological Basis of Therapeutics.

DM is a non-addictive opioid comprising a dextrorotatory enantiomer (mirror image) of the morphinan ring structure which forms the molecular core of most opiates. DM acts at a class of neuronal receptors known as sigma receptors. These are often referred to as sigma opiate receptors, but there is some question as to whether they are opiate receptors, so many researchers refer to them simply as sigma receptors, or as high-affinity dextromethorphan receptors. They are inhibitory receptors, which mean that their activation by DM or other sigma agonists causes the suppression of certain types of nerve signals.

Dextromethorphan also acts at another class of receptors known as N-methyl-D-aspartate (NMDA) receptors, which are one type of excitatory amino acid (EAA) receptor. Unlike its agonist activity at sigma receptors, DM acts as an antagonist at NMDA receptors, which means that DM suppresses the transmission of nerve impulses mediated via NMDA receptors. Since NMDA receptors are excitatory receptors, the activity of DM as an NMDA antagonist also leads to the suppression of certain types of nerve signals, which may also be involved in some types of coughing.

Due to its activity as an NMDA antagonist, DM and one of its metabolites, dextrorphan, are being actively evaluated as possible treatments for certain types of excitotoxic brain damage caused by ischemia (low blood flow) and hypoxia (inadequate oxygen supply), which are caused by events such as stroke, cardiac arrest, and asphyxia. The anti-excitotoxic activity of dextromethorphan and dextrorphan, and the blockade of NMDA receptors by these drugs, are discussed in items such as Choi (Choi D W. Dextrorphan and dextromethorphan attenuate glutamate neurotoxicity. Brain Res 1987; 403: 333-6), and, Steinberg et al (Steinberg G K et al, Delayed treatment with dextromethorphan and dextrorphan reduces cerebral damage after transient focal ischemia, Neurosci Letters 1988; 89: 193-197). Dextromethorphan has also been reported to suppress activity at neuronal calcium channels (Carpenter C L, et al, Dextromethorphan and dextrorphan as calcium channel antagonists, Brain Research 1988; 439: 372-375). Further, both DM and dextrorphan are both 5-HT and NE Reuptake inhibitors (Table 1).

TABLE 1
Receptor Binding Affinity and Reuptake Inhibition of
Dextromethorphan, Tramadol, Gabapentin and their Metabolites

| | Receptors and Binding Constant Kᵢ (nM) | | | | | | |
|---|---|---|---|---|---|---|---|
| Compound | μ-opioid | δ-opioid | κ-opioid | NE | 5-HT | NMDA | Sigma |
| Dextromethorphan | 1280 | 11500 | 7000 | 240 | 23 | 2913 | 365 |
| Dextrorphan | 420 | 34,700 | 5,950 | 340 | 401 | 450 | 559 |
| (±) Tramadol | 2120 | 57,700 | 42,700 | 785 | 992 | >100,000 | |
| (±) M1 | 12.1 | 911 | 242 | 1520 | 5180 | >16,000 | |
| (O-desmethyl | | | | | | | |
| Tramadol) | | | | | | | |
| (−) Morphine | 1.24 | 145 | 23.4 | | | | |
| Naltrexone | 0.08 | 8.02 | 0.51 | | | | |
| Levorphanol | 0.42 | 3.61 | 4.2 | 1210 | 86.3 | | |
| MK-801 | | | | | | 0.5 (high) | |
| | | | | | | 77 (low) | |
| Paroxetine | | | | | 0.12 | | |
| Chlorpheniramine | | | | | 510 | | |
| Venlafaxine | | | | | 39.0 | | |
| Milnacipran | | | | 300 | 100 | 6300 | |

| | |
|---|---|
| Gabapentin | α2δ Binding (59 nM) |

### SOURCE

Raffa et al., J Pharmacol Exp Ther 260:275-85, 1992
Raffa et al., J Pharmacol Exp Ther 267:331-40, 1993
Codd E E, Shank P R, Schupsky J J, and Raffa R B. Serotonin and Norepinephrine Uptake Inhibiting Activity of Centrally Acting Analgesics: Structural Determinants and Role in Antinociception. JPET 274: 1263-1270, 1995
Palmer G C. Neuroprotection by NMDA Receptor Antagonists in a Variety of Neuropathologies. Current Drug Targets, 2001; 2, 241-271
Hara K, Minami K, and Sata T. The Effects of Tramadol and Its Metabolite on Glycine, Alpha-AminobutyricAcid, and N-Methyl-D-Aspartate Receptors Expressed in Xenopus Oocytes. Anesth Analg 2005; 100: 1400-5.
Hyttel J, Comparative pharmacology of selective serotonin reuptake inhibitors (SSRIs). Nord J Psychiatry. 1993; 47 (suppl 30):5-12.
Meade C J. The Mechanism by Which Epinastine Stops an Adenosine Analog from Contracting BDE Rat Airways. Am J Respir Crit Care Med 1998; 157: 522-530.
Muth E A, Moyer J A, Haskins J T, Andree T H, Husbands G E M. Biochemical, neurophysiological, and behavioral effects of Wy-45,233 and other identified metabolites of the antidepressant venlafaxine. Drug Dev Res. 1991; 23:191-199.
Bolden-Watson C, Richelson E. Blockade by newly-developed antidepressants of biogenic amine uptake into rat brain synaptosomes. Life Sci. 1993; 52:1023-1029.
Marais E, Klugbauer N, Hofmann F. Calcium channel alpha(2)delta subunits-structure and Gabapentin binding. Mol Pharmacol. 2001 May; 59(5):1243-8.
Tatsuro K, et al., Milnacipran inhibits glutamatergic N-Methyl-D-Aspartate receptor activity in Spinal Dorsal Horn Neurons. Molecular Pain 2012, 8:45-53.

DM disappears fairly rapidly from the bloodstream (see, e.g., Vettican S J et al., Phenotypic differences in dextromethorphan metabolism, Pharmaceut Res 1989; 6: 13-19). DM is converted in the liver to two metabolites called dextrorphan and 3-methoxymorphinan, by an enzymatic process called O-demethylation; in this process, one of the two pendant methyl groups is replaced by hydrogen. If the second methyl group is removed, the resulting metabolite is called 5-hydroxymorphinan. Dextrorphan and 5-hydroxymorphinan are covalently bonded to other compounds in the liver (primarily glucuronic acid or sulfur-containing compounds such as glutathione) to form glucuronide or sulfate conjugates which are eliminated fairly quickly from the body via urine bloodstream.

In summary, Dextromethorphan and its active metabolite dextrorphan bind to the N-Methyl-D-Aspartate (NMDA) glutamate and nicotine/neuronal nicotinic receptors as inhibitors. Dextromethorphan and dextrorphan also bind to the receptor-gated (NMDA receptor mediated) and voltage-gated calcium channels, and the voltage-gated sodium channels as a blocker. Through these bindings, dextromethorphan and dextrorphan modulates the glutamate pathway in the central nervous system (CNS) and modulate most of the excitatory synaptic transmission. Dextromethorphan and dextrorphan also bind to the sigma receptors which are found in high concentrations in limbic and motor areas of the CNS sensory processing such as the dorsal root ganglia and the nucleus tractus solitarus (NTS). In addition, Dextromethorphan inhibits the reuptake of 5-HT (serotonin) and norepinephrine, thus modulating the monamine pathways.

Dextromethorphan is typically administered orally. As an antitussive, the recommended dosage for adults is 60-120 mg daily in divided doses. Each current FDA approved brand contains different quantities of dextromethorphan, generally 20-30 mg per dose. Approximate doses are: threshold dose 80-90 mg; light 100-200 mg; common 200-400 mg; strong 400-600 mg; and heavy dose 600-1500 mg.

At recommended doses, dextromethorphan produces little or no CNS depression. At higher doses, positive effects may include acute euphoria, elevated mood, dissociation of mind from body, creative dream-like experiences, and increased perceptual awareness. Other effects include disorientation, confusion, pupillary dilation, and altered time perception, visual and auditory hallucinations, and decreased sexual functioning. Doses of approximately 100-200 mg have a mild, stimulant effect (likened to MDA); doses of 200-500 mg produce a more intoxicating effect (likened to being ‘drunk and stoned’); 500-1000 mg may result in mild hallucinations and a mild dissociate effect (likened to a low dose of ketamine) and an overall disturbance in thinking, senses and memory; while doses over 1000 mg may produce a fully dissociative effect (likened to a high dose of ketamine). Abused doses are capable of impairing judgment, memory, language, and other mental performances.

Tramadol has the chemical name (+/−)-trans(RR,SS)-2-[(di-methylamino)methyl]-1-(3-methoxyphenyl)cyclohexanol, and which is often erroneously referred to in literature as the cis(RS,SR) diastereomer (FIG. 2). Tramadol is a centrally acting, binary analgesic that is neither opiate-derived, nor is it an NSAID. It is used to control moderate pain in chronic pain settings, such as osteoarthritis and post-operative analgesia, and acute pain, such as dental pain.

Tramadol is a racemate and consists of equal quantities of (+)- and (−)-enantiomers. It is known that the pure enantiomers of tramadol have a differing pharmaceutical profiles and effects when compared to the racemate. The (+)-enantiomer is distinguished by an opiate-like analgesic action due its binding with the μ-opiate receptor, and both enantiomers inhibit 5-hydroxytryptamine (serotonin) and noradrenaline (norepinephrine) reuptake, which is stronger than that of racemic mixtures of tramadol, while distinct inhibition of noradrenaline reuptake is observed with the (−)-enantiomer.

It has been proven for (+)- and (−)-tramadol that, depending upon the model, the two enantiomers mutually reinforce and enhance their individual actions (Raffa R B, et al. Complementary and synergistic antinociceptive interaction between the enantiomers of tramadol J Pharmacol Exp Ther 1993; 267: 331-40). It is obvious to conclude that the potent analgesic action of tramadol is based on this mutually dependent reinforcement of action of the enantiomers. Tramadol's major active metabolite, O-desmethyltramadol (M1), shows higher affinity for the μ-opiate receptor and has at least twice the analgesic potency of the parent drug. O-desmethyl-N-mono-desmethyltramadol (referred to as M5 in some places in the following text and in the literature) is known as one of the in vivo metabolites of tramadol (1RS,2RS)-2[(dimethylamino)methyl]-1-(3-methoxyphenyl)cyclohexanol). M5 penetrates the blood-brain barrier to only a limited extent, as the effects on the central nervous system, for example analgesic effects, are distinctly less pronounced on intravenous administration than on intracerebroventricular administration. Despite the fact that Tramadol is chemically unrelated to the opiates adverse side effects associated with administration of Tramadol are similar to those of the opiates if used at higher doses. The binding of Tramadol and M1 with various receptors are shown in Table 1.

In summary, Tramadol and its active metabolite M1, modulate neuronal pathways via contributions from both opioid (predominantly at the n-opioid receptor) and non-opioid probably related to its inhibition of neuronal release or reuptake of norepinephrine and serotonin) mechanisms at therapeutic doses. Both mechanisms contribute to the effect of tramadol in vivo, leading to the suggestion that tramadol is a novel centrally acting analgesic that mimics, in a single drug substance, the clinical practice of combining opioid analgesics with monoamine reuptake inhibitors. Opioid receptors presynaptically inhibit transmission of excitatory pathways. These pathways include acetylcholine, the catecholamines, serotonin, and substance P. The present working hypothesis is that the overall neuronal action of tramadol is dependent on the different pharmacologies of its enantiomers and, to some extent its metabolite, M1. The enantiomers appear to interact in a complementary and synergistic manner to produce antinociception, but only in an additive or counteractive manner on adverse-effect end-points. Hence, the favorable clinical profile of tramadol appears to be a consequence of the fortuitous interaction of the enantiomers and the metabolite M1 on the therapeutic endpoint, but not on adverse-effect endpoints.

Tramadol has been given is single oral doses of 50, 75, and 100 mg to patients with pain following surgical procedures and pain following oral surgery. In single-dose models of pain following oral surgery, pain relief was demonstrated in some patients at doses of 50 and 75 mg. A dose of 100 mg tended to provide analgesia superior to codeine sulfate 60 mg, but it was not as effective as the combination of aspirin 650 mg with codeine phosphate 60 mg.

Tramadol has been studied in three long-term controlled trials involving a total of 820 patients, with 530 patients receiving tramadol. Patients with a variety of chronic painful conditions were studied in double-blind trials of one to three months duration. The average daily doses of approximately 250 mg tramadol in divided doses were generally comparable to five doses of acetaminophen 300 mg with codeine phosphate 30 mg (T#3) daily, five doses of aspirin 325 mg with codeine phosphate 30 mg daily, or two to three doses of acetaminophen 500 mg with oxycodone hydrochloride 5 mg daily. Tramadol 50 to 100 mg can be administered in adults over 17 years of age as needed for pain relief every 4 to 6 hours not to exceed 400 mg per day.

The recommended daily dose of tramadol for treating neuropathic pain is between 50 and 100 mg every 4 to 6 hours, with a maximum dose of 400 mg/day. The duration of the analgesic effect after a single oral dose of tramadol 100 mg is about 6 hours. Adverse effects, and nausea in particular, are dose dependent and therefore considerably more likely to appear if the loading dose is high. The reduction of this dose during the first days of treatment is an important factor in improving tolerability. Other adverse effects are generally similar to those of opiates, although they are usually less severe, and can include respiratory depression, dysphoria and constipation.

Gabapentin (FIG. 3) is structurally related to the neurotransmitter GABA (gamma-aminobutyric acid) but it does not modify GABA_{A} or GABA_{B} radioligand binding, it is not converted metabolically into GABA or a GABA agonist, and it is not an inhibitor of GABA uptake or degradation. Gabapentin was tested in radioligand binding assays at concentrations up to 100 μM and did not exhibit affinity for a number of other common receptor sites, including benzodiazepine, glutamate, N-methyl-D-aspartate (NMDA), quisqualate, kainate, strychnine-insensitive or strychnine-sensitive glycine, α1, α2, or β-adrenergic, adenosine A1 or A2, cholinergic muscarinic or nicotinic, dopamine D1 or D2, histamine H1, serotonin S1 or S2, opiate μ, δ or κ, cannabinoid 1, voltage-sensitive calcium channel sites labeled with nitrendipine or diltiazem, or at voltage-sensitive sodium channel sites labeled with batrachotoxinin A 20-α-benzoate. Furthermore, gabapentin did not alter the cellular uptake of dopamine, noradrenaline, or serotonin. It has been shown that gabapentin presynaptically inhibits glutamate transmission and that gabapentin antagonizes AMPA-evoked responses in vivo. Furthermore, a study in trigeminal nucleus slices showed that glutamate release activated by protein kinase C is blocked by gabapentin.

Gabapentin is known to interact with both the α_{2δ-}1 and α_{2δ-}2 subunits which are voltage-gated calcium channel thus blocking calcium influx into the neuronal cells (Table 1). A specific role for α₂δ in neuropathic pain is due to the fact that an increase in α₂δ expression in the dorsal root ganglion ipsilateral to the peripheral nerve injury that corresponded to the development of tactile allodynia. In addition, gabapentin has been shown to increase brain extracellular GABA levels in both rat and human studies which is partially responsible for its effectiveness for neuropathic pain, since the pathology associated with this condition includes disruption of tonic inhibitory GABAergic transmission.

In summary, Gabapentin interacts with both the α_{2δ-}1 and α_{2δ-}2 subunits which are voltage-gated calcium channel thus blocking calcium influx into the neuronal cells. A specific role for α₂δ in neuropathic pain is due to the fact that an increase in α₂δ expression in the dorsal root ganglion ipsilateral to the peripheral nerve injury that corresponded to the development of tactile allodynia. In addition, gabapentin increases brain extracellular GABA levels in both rat and human studies which is partially responsible for its effectiveness for neuropathic pain, since the pathology associated with this condition includes disruption of tonic inhibitory GABAergic transmission.

Gabapentin is commercially supplied as Neurontin® Capsules, Neurontin Tablets, and Neurontin Oral Solution, as imprinted hard shell capsules containing 100 mg, 300 mg, and 400 mg of gabapentin, elliptical film-coated tablets containing 600 mg and 800 mg of gabapentin or an oral solution containing 250 mg/5 mL of gabapentin. Gabapentin bioavailability is not dose proportional; i.e., as dose is increased, bioavailability decreases. Bioavailability of gabapentin is approximately 60%, 47%, 34%, 33%, and 27% following 900, 1200, 2400, 3600, and 4800 mg/day given in 3 divided doses, respectively. Food has only a slight effect on the rate and extent of absorption of gabapentin (14% increase in AUC and Cₘₐₓ). Less than 3% of gabapentin circulates bound to plasma protein. The apparent volume of distribution of gabapentin after 150 mg intravenous administration is 58±6 L (Mean±SD). In patients with epilepsy, steady-state predose (Cₘᵢₙ) concentrations of gabapentin in cerebrospinal fluid were approximately 20% of the corresponding plasma concentrations. Gabapentin is eliminated from the systemic circulation by renal excretion as unchanged drug. Gabapentin is not appreciably metabolized in humans. Gabapentin elimination half-life is 5 to 7 hours and is unaltered by dose or following multiple dosing. Gabapentin elimination rate constant, plasma clearance, and renal clearance are directly proportional to creatinine clearance. In elderly patients, and in patients with impaired renal function, gabapentin plasma clearance is reduced. Gabapentin can be removed from plasma by hemodialysis.

Currently gabapentin is indicated as adjunctive therapy in the treatment of partial seizures with and without secondary generalization in patients over 12 years of age with epilepsy. Gabapentin is also indicated as adjunctive therapy in the treatment of partial seizures in pediatric patients age 3¾-12 years.

Gabapentin is not appreciably metabolized nor does it interfere with the metabolism of commonly coadministered antiepileptic drugs. Gabapentin is given orally with or without food. In adults with postherpetic neuralgia, Gabapentin therapy may be initiated as a single 300-mg dose on Day 1, 600 mg/day on Day 2 (divided BID), and 900 mg/day on Day 3 (divided TID). The dose can subsequently be titrated up as needed for pain relief to a daily dose of 1800 mg (divided TID). In clinical studies, efficacy was demonstrated over a range of doses from 1800 mg/day to 3600 mg/day with comparable effects across the dose range. Additional benefit of using doses greater than 1800 mg/day was not demonstrated. For patients >12 years of age: The effective dose of gabapentin is 900 to 1800 mg/day and given in divided doses (three times a day) using 300 or 400 mg capsules, or 600 or 800 mg tablets. The starting dose is 300 mg three times a day. If necessary, the dose may be increased using 300 or 400 mg capsules, or 600 or 800 mg tablets three times a day up to 1800 mg/day. Dosages up to 2400 mg/day have been well tolerated in long-term clinical studies. Doses of 3600 mg/day have also been administered to a small number of patients for a relatively short duration, and have been well tolerated. The maximum time between doses in the TID schedule should not exceed 12 hours.

The most commonly observed adverse events associated with the use of gabapentin in adults, not seen at an equivalent frequency among placebo-treated patients, were dizziness, somnolence, and peripheral edema. The most commonly observed adverse events associated with the use of gabapentin in combination with other antiepileptic drugs in patients >12 years of age, not seen at an equivalent frequency among placebo-treated patients, were somnolence, dizziness, ataxia, fatigue, and nystagmus. The most commonly observed adverse events reported with the use of gabapentin in combination with other antiepileptic drugs in pediatric patients 3 to 12 years of age, not seen at an equal frequency among placebo-treated patients, were viral infection, fever, nausea and/or vomiting, somnolence, and hostility.

In adults with postherpetic neuralgia, gabapentin therapy may be initiated as a single 300-mg dose on Day 1, 600 mg/day on Day 2 (divided BID), and 900 mg/day on Day 3 (divided TID). The dose can subsequently be titrated up as needed for pain relief to a daily dose of 1800 mg (divided TID). In clinical studies, efficacy was demonstrated over a range of doses from 1800 mg/day to 3600 mg/day with comparable effects across the dose range. Additional benefit of using doses greater than 1800 mg/day was not demonstrated. For patients >12 years of age: The effective dose of Neurontin is 900 to 1800 mg/day and given in divided doses (three times a day) using 300 or 400 mg capsules, or 600 or 800 mg tablets. The starting dose is 300 mg three times a day. If necessary, the dose may be increased using 300 or 400 mg capsules, or 600 or 800 mg tablets three times a day up to 1800 mg/day. Dosages up to 2400 mg/day have been well tolerated in long-term clinical studies. Doses of 3600 mg/day have also been administered to a small number of patients for a relatively short duration, and have been well tolerated. The maximum time between doses in the TID schedule should not exceed 12 hours.

The most commonly observed adverse events associated with the use of gabapentin in adults, not seen at an equivalent frequency among placebo-treated patients, were dizziness, somnolence, and peripheral edema. The most commonly observed adverse events associated with the use of gabapentin in combination with other antiepileptic drugs in patients >12 years of age, not seen at an equivalent frequency among placebo-treated patients, were somnolence, dizziness, ataxia, fatigue, and nystagmus. The most commonly observed adverse events reported with the use of gabapentin in combination with other antiepileptic drugs in pediatric patients 3 to 12 years of age, not seen at an equal frequency among placebo-treated patients, were viral infection, fever, nausea and/or vomiting, somnolence, and hostility.

Pregabalin, an analog of gabapentin, is sold commercially as LYRICA capsules and is administered orally and are supplied as imprinted hard-shell capsules containing 25, 50, 75, 100, 150, 200, 225, and 300 mg of pregabalin, along with lactose monohydrate, cornstarch, and talc as inactive ingredients. The capsule shells contain gelatin and titanium dioxide. In addition, the orange capsule shells contain red iron oxide and the white capsule shells contain sodium lauryl sulfate and colloidal silicon dioxide. Colloidal silicon dioxide is a manufacturing aid that may or may not be present in the capsule shells. The imprinting ink contains shellac, black iron oxide, propylene glycol, and potassium hydroxide.

Treatment with pregabalin 100 and 200 mg three times a day statistically significantly improved the endpoint mean pain score and increased the proportion of patients with at least a 50% reduction in pain score from baseline. There was no evidence of a greater effect on pain scores of the 200 mg three times a day dose than the 100 mg three times a day dose, but there was evidence of dose dependent adverse reactions. A 13-week study compared pregabalin 75, 150, and 300 mg twice daily with placebo. Patients with creatinine clearance (CLcr) between 30 to 60 mL/min were randomized to 75 mg, 150 mg, or placebo twice daily. Patients with creatinine clearance greater than 60 mL/min were randomized to 75 mg, 150 mg, 300 mg or placebo twice daily. In patients with creatinine clearance greater than 60 mL/min treatment with all doses of pregabalin statistically significantly improved the endpoint mean pain score and increased the proportion of patients with at least a 50% reduction in pain score from baseline. Despite differences in dosing based on renal function, patients with creatinine clearance between 30 to 60 mL/min tolerated pregabalin less well than patients with creatinine clearance greater than 60 mL/min as evidenced by higher rates of discontinuation due to adverse reactions.

An 8-week study compared pregabalin 100 or 200 mg three times a day with placebo, with doses assigned based on creatinine clearance. Patients with creatinine clearance between 30 to 60 mL/min were treated with 100 mg three times a day, and patients with creatinine clearance greater than 60 mL/min were treated with 200 mg three times daily. Treatment with pregabalin statistically significantly improved the endpoint mean pain score and increased the proportion of patients with at least a 50% reduction in pain score from baseline.

A 8-week study compared pregabalin 50 or 100 mg three times a day with placebo with doses assigned regardless of creatinine clearance. Treatment with pregabalin 50 and 100 mg three times a day statistically significantly improved the endpoint mean pain score and increased the proportion of patients with at least a 50% reduction in pain score from baseline. Patients with creatinine clearance between 30 to 60 mL/min tolerated pregabalin less well than patients with creatinine clearance greater than 60 mL/min as evidenced by markedly higher rates of discontinuation due to adverse reactions.

A 14-week study compared pregabalin total daily doses of 300 mg, 450 mg and 600 mg with placebo. Patients were enrolled with a minimum mean baseline pain score of greater than or equal to 4 on an 11-point numeric pain rating scale and a score of greater than or equal to 40 mm on the 100 mm pain visual analog scale (VAS). The baseline mean pain score in this trial was 6.7. Responders to placebo in an initial one-week run-in phase were not randomized into subsequent phases of the study. A total of 64% of patients randomized to pregabalin completed the study. There was no evidence of a greater effect on pain scores of the 600 mg daily dose than the 450 mg daily dose, but there was evidence of dose-dependent adverse reactions.

The maximum recommended dose of pregabalin for neuropathic pain associated with diabetic peripheral neuropathy is 100 mg three times a day (300 mg/day) in patients with creatinine clearance of at least 60 mL/min. Dosing should begin at 50 mg three times a day (150 mg/day) and may be increased to 300 mg/day within 1 week based on efficacy and tolerability. Because pregabalin is eliminated primarily by renal excretion, the dose should be adjusted for patients with reduced renal function. Although pregabalin was also studied at 600 mg/day, there is no evidence that this dose confers additional significant benefit and this dose was less well tolerated. In view of the dose-dependent adverse reactions, treatment with doses above 300 mg/day is not recommended.

The recommended dose of pregabalin for fibromyalgia is 300 to 450 mg/day. Dosing should begin at 75 mg two times a day (150 mg/day) and may be increased to 150 mg two times a day (300 mg/day) within 1 week based on efficacy and tolerability. Patients who do not experience sufficient benefit with 300 mg/day may be further increased to 225 mg two times a day (450 mg/day). Although pregabalin was also studied at 600 mg/day, there is no evidence that this dose confers additional benefit and this dose was less well tolerated. In view of the dose-dependent adverse reactions, treatment with doses above 450 mg/day is not recommended. Because pregabalin is eliminated primarily by renal excretion, the dose should be adjusted for patients with reduced renal function (creatinine clearance less than 60 mL/min—see Patients with Renal Impairment).

In clinical trials in patients with neuropathic pain associated with diabetic peripheral neuropathy, 9% of patients treated with pregabalin and 4% of patients treated with placebo discontinued prematurely due to adverse reactions. In the pregabalin treatment group, the most common reasons for discontinuation due to adverse reactions were dizziness (3%) and somnolence (2%). In comparison, <1% of placebo patients withdrew due to dizziness and somnolence. Other reasons for discontinuation from the trials, occurring with greater frequency in the pregabalin group than in the placebo group, were asthenia, confusion, and peripheral edema. Each of these events led to withdrawal in approximately 1% of patients.

In clinical trials of patients with fibromyalgia, 19% of patients treated with pregabalin (150-600 mg/day) and 10% of patients treated with placebo discontinued prematurely due to adverse reactions. In the pregabalin treatment group, the most common reasons for discontinuation due to adverse reactions were dizziness (6%) and somnolence (3%). In comparison, <1% of placebo-treated patients withdrew due to dizziness and somnolence. Other reasons for discontinuation from the trials, occurring with greater frequency in the pregabalin treatment group than in the placebo treatment group, were fatigue, headache, balance disorder, and weight increased. Each of these adverse reactions led to withdrawal in approximately 1% of patients.

Over 325 enzymes are magnesium dependent with many being brain enzymes. Magnesium deficiency modifies the turnover of various types of neurotransmitters including amino acids, nitric oxide, neuropeptides and cytokines (Durlach J, Bac P. Mechanisms of action on the nervous system in magnesium deficiency and dementia. In: Yasui M, Strong M J, Ota K, Verity M A, editors. Mineral and metal neurotoxicology. Boca Raton, N.Y.: CRC Press; 1997. p. 201-9). Intracellular effects of Mg2+ ions are mainly opposite to those of Ca2+ ions, possibly owing to competition at sites where Ca2+ ions activate K+ ion channels. Magnesium-deficiency produces epileptiform activity in the CNS which can be blocked by NMDA-receptor antagonists. Other mechanisms, including alterations in Na+/K(+)-ATPase activity, cAMP/cGMP concentrations and calcium currents in pre- and postsynaptic membranes, may also be at least partially responsible for the neuronal injury associated with low brain magnesium and depression (Morris M E. Brain and CSF magnesium concentrations during magnesium deficit in animals and humans: neurological symptoms. Magnes Res 1992; 5:303-13).

Magnesium-deficiency was found to cause numerous neurological and neuromuscular symptoms including hyperexcitability, depression, pain, behavior disturbances, tetany, headaches, focal seizures, ataxia, anxiety, vertigo, muscular weakness, tremors, irritability, and psychotic behavior, each of which were reversible by magnesium repletion. Hypomagnesemia was also seen in patients with various diseases such as cancer, hepatic cirrhosis, cardiovascular, cerebrovascular disease, and generally poor condition. The most common clinical findings of hypomagnesemia were personality changes and major depression showing that differentiation of brain hypomagnesemia from psychiatric disease is important (Hashizume N, Mori M. An analysis of hypermagnesemia and hypomagnesemia. Jpn J Med 1990; 29:368-72). Deficits of magnesium result due to inadequate intake or malabsorption and due to the renal loss of magnesium that occurs in certain disease states alcoholism, diabetes) and with drug therapy (antidiuretics, aminoglycosides, fluoroquinolones, cisplatin, digoxin, cyclosporin, amphotericin B)

Most of the brain's regular functions operate quickly and involve the excitatory amino acids glutamate and aspartate in the NMDA receptors. They are involved in NMDA nerve cell electrical conduction activity across brain cell synapses. Learning (long-term potentiation), memory and depression have their foundation in NMDA receptors. Magnesium-depletion is specifically deleterious to neurons by causing NMDA-coupled calcium channels to be biased towards opening (Sapolsky R M. Stress the aging brain and the mechanisms of neuron death. Cambridge, Mass.: A Bradford Book, The MIT Press; 1992. p. 192), because magnesium is nature's calcium channel blocker (Iseri L T, French J H. Magnesium: nature's physiologic calcium blocker. Am Heart J 1984; 108:188-93). The targets for glutamate binding to NMDA receptors are calcium and magnesium ion channels and to a lesser extent calcium and zinc channels (Mark L P, et al. Pictorial review of glutamate excitotoxicity: fundamental concepts for neuroimaging. AJNR Am J Neuroradiol 2001; 22:1813-24). At normal neuronal resting membrane potentials, pores of the glutamate-gated ion channel are blocked by Mg2+ ions. The ion channel of the NMDA-receptor complex is subject to voltage-dependent regulation by magnesium ions (Decollogne S, et al. NMDA receptor complex blockade by oral administration of magnesium: comparison with MK-801. Pharmacol Biochem Behav 1997; 58:261-8). Normally operating NMDA receptors admit into neurons only the amount of Ca2+ that is vital to their function, but abnormally functioning NMDA receptors increase influx of cellular Ca2+ beyond manageable levels leading to the generation of toxic reactive oxygen species and of toxic amounts of nitric oxide (NO) radicals (Carafoli E. Calcium—a universal carrier of biological signals. FEBS J 2005; 272:1073-89). It has been shown that NMDA receptor channel characteristics in the dorsal horn are altered by inflammation, and that the changes observed could contribute to the hyperalgesia and allodynia associated with tissue injury (H Guo, L Y Huang. Alteration in the voltage dependence of NMDA receptor channels in rat dorsal horn, J Physiol (2001) 537: 115-23). Imbalances in Na+ and Cl⁻ gradients as well as Ca2+ overloading are also implicated in neuronal swelling and cell death (Gillessen T, et al. Excitatory amino acid neurotoxicity. In: Alzheimer C, editor. Molecular and cellular biology of neuroprotection in the CNS series: advances in experimental medicine and biology, vol. 513. New York, N.Y.: Kluwer Academic/Plenum Publishers, Georgetown, Tex.: Landes Bioscience; 2002; 3-40), while depolarization of membranes relieves the Mg2+ block and allows Na+ and Ca2+ to enter. Certain drugs can act in place of magnesium including memantine and ketamine (Hollmann M W, et al. Modulation of NMDA receptor function by ketamine and magnesium part II: interactions with volatile anesthetics. Anesth Analg 2001; 92:1182-91) with each producing benefits in depression.

It has been shown that the use of Intravenous magnesium (30-mg/kg bolus, 10 mg/kg/h infusion for 48 hours) reduced the use of narcotics in postoperative patients (Perihan E E, et al. Role of magnesium sulfate in postoperative pain management for patients undergoing thoracotomy. Journal of cardiothoracic and vascular anesthesia, 2007; 21, 827-831). In almost all the clinical studies involving pain, very high doses of magnesium were required to have any meaningful reduction in pain. These high doses could be used for chronic pain treatment.

In summary, Magnesium-depletion is specifically deleterious to neurons by causing NMDA-coupled calcium channels to be biased towards opening, because magnesium is nature's calcium channel blocker. The targets for glutamate binding to NMDA receptors are calcium and magnesium ion channels and to a lesser extent calcium and zinc channels. At normal neuronal resting membrane potentials, pores of the glutamate-gated ion channel are blocked by Mg2+ ions. The ion channel of the NMDA-receptor complex is subject to voltage-dependent regulation by magnesium ions. Normally operating NMDA receptors admit into neurons only the amount of Ca2+ that is vital to their function, but abnormally functioning NMDA receptors increase influx of cellular Ca2+ beyond manageable levels leading to the generation of toxic reactive oxygen species and of toxic amounts of nitric oxide (NO) radicals. It has been shown that NMDA receptor channel characteristics in the dorsal horn are altered by inflammation, and that the changes observed could contribute to the hyperalgesia and allodynia associated with tissue injury. Imbalances in Na+ and Cl⁻ gradients as well as Ca2+ overloading are also implicated in neuronal swelling and cell death, while depolarization of membranes relieves the Mg2+ block and allows Na+ and Ca2+ to enter. Certain drugs can act in place of magnesium including memantine, dextromethorphan and ketamine with each producing benefits in depression.

Venlafaxine is a novel SSRI chemically unrelated to other SSRIs but chemically similar to the tramadol (FIG. 4; Markowitz J S, Patrick K S. Venlafaxine-tramadol similarities. Medical Hypotheses 1998; 51:167-8). The chemical structures of venlafaxine and tramadol are similar, demonstrating the similarity between these two antidepressant and analgesic substances, respectively. It is designated (R/S)-1-[2-(dimethylamino)-1-(4-methoxyphenyl)ethyl]cyclohexanol or (±)-1-[a-[(dimethylamino)methyl]-p-methoxybenzyl]cyclohexanol and has the empirical formula of C₁₇H₂₇NO₂. Venlafaxine hydrochloride (Effexor) is formulated as capsule for oral administration. Capsules contain venlafaxine hydrochloride equivalent to 37.5 mg, 75 mg, or 150 mg venlafaxine.

The mechanism of the antidepressant action of venlafaxine in humans is believed to be the same as with other SSRIs, associated with its potentiation of neurotransmitter activity in the CNS as with other SSRIs: preclinical studies have shown that venlafaxine and its active metabolite, O-desmethylvenlafaxine (ODV), are potent inhibitors of neuronal serotonin and norepinephrine reuptake and weak inhibitors of dopamine reuptake. That venlafaxine is analgesic is seen in studies in animals that show that venlafaxine is effective in reversing chronic neuropathic pain secondary to thermal hyperalgesia, and additionally is effective in treating the hyperalgesia of neuropathic pain due to chronic sciatic nerve constriction injury in rats (Lang E, et al. Venlafaxine hydrochloride (Effexor) relieves thermal hyperalgesia in rats with an experimental mononeuropathy. Pain 1998; 68:151-5). Studies have shown that, the antinociceptive effect of venlafaxine is mainly influenced by the κ- and δ-opioid receptor subtypes combined with the α2-adrenergic receptor. These results suggest a potential use of venlafaxine in the management of some pain syndromes.

Tricyclic antidepressants may be useful as adjunctive therapy for cancer-related neuropathic pain syndromes (Jacox A, et al. Clinical practice guideline number 9: management of cancer pain. Rockville, Md.: U.S. Department of Health and Human Services, Agency for Health Care Policy and Research, 1994. AHCPR publication no. 94-0592). Tricyclic antidepressants provide pain relief by independently providing analgesia specific for neuropathic pain, potentiating the effect of opioids, and improving underlying depression and insomnia (Levy M H. Pharmacologic treatment of cancer pain. N Engl J Med 1996; 335:1124-32). Although controlled trials using tricyclic anti-depressants in patients with cancer are limited and most data about tricyclic antidepressant analgesic effectiveness have been obtained with other chronic pain syndromes, these agents are accepted as adjunctive analgesics for cancer pain.

Tricyclic antidepressants should be administered cautiously in patients with angle-closure glaucoma, benign prostatic hypertrophy, urinary retention, constipation, cardiovascular disease, or impaired liver function. The agents should be avoided in patients with second- or third-degree heart block, arrhythmias, prolonged QT interval on the electrocardiogram, or severe liver disease and in patients who have had a recent acute myocardial infarction.

The adverse effects of tricyclic antidepressants are well known, but their prevalence rates vary by agent and patient group. In general, elderly patients experience a higher frequency of adverse effects, and slow dosage titration is recommended (Rudorfer M V, et al. Comparative tolerability profiles of the newer versus older antidepressants. Drug Saf 1994; 10:18-46). The most common adverse effects of tricyclic antidepressants (constipation, dry mouth, blurred vision, cognitive changes, tachycardia, urinary hesitation) are associated with their anticholinergic activity. Other common adverse effects are orthostatic hypotension, falls, weight gain, and sedation. In general, the secondary amines (e.g., desipramine, nortriptyline) exhibit fewer anticholinergic and sedative effects than do the tertiary amines (e.g., amitriptyline, imipramine, doxepin); therefore, the secondary amines may be more desirable in the elderly population (Lipman A G. Analgesic drugs for neuropathic and sympathetically maintained pain. Clin Geriatr Med 1996; 12:501-15).

Not all patients respond to tricyclic anti-depressant therapy within 10 days of initiation or with lower dosages. Some patients may require higher dosages and several weeks of treatment before efficacy is evident. Patients are often referred to specialty pain clinics because the tricyclic antidepressant dosage was not adequate. In addition, these drugs may be discontinued unnecessarily because of adverse effects caused by starting them at inappropriately high dosages, titrating the dosage upward too rapidly, or starting several drugs at one time (Galer B S. Painful polyneuropathy: diagnosis, patho-physiology, and management. Semin Neurol 1994; 14:237-46). An adequate trial must be given before failure of a tricyclic antidepressant is determined. Failure of one tricyclic antidepressant does not preclude success with a different agent, and the practitioner should consider trying two or, perhaps, three agents sequentially before substituting another therapeutic option.

Patients abruptly withdrawn from a tricyclic antidepressant may experience withdrawal that manifests as any of a variety of clinical symptoms (e.g., malaise, insomnia, drowsiness, anorexia, muscle aches, apathy, headache, mania, profuse sweating, irritability, abdominal pains, diarrhea, nausea, vivid and terrifying dreams, movement disorders). To avoid a withdrawal syndrome, a slow taper over 2-4 weeks (depending on the dosage) is recommended (Garner E M, et al. Tricyclic antidepressant withdrawal syndrome. Ann Pharmacother 1993; 27:1068-72).

Amitriptyline is a tricyclic agent used for the treatment of major depression (FIG. 5; Baldessarini R J (1995) Drugs and the treatment of psychiatric disorders, in The Pharmacological Basis of Therapeutics (Hardman J G, Limbird L E, Molinoff P B, Ruddon R W and Gilman A G eds) pp 431-459, McGraw-Hill, New York). Amitriptyline, nortriptyline, and de sipramine have been established as analgesics independent of their antidepressant effects. Although their mechanism of analgesic action has not been clearly defined, tricyclic antidepressants are thought to have an inhibitory effect on nociceptive pathways by blocking the reuptake of serotonin and norepinephrine (Calissi P T, Jaber L A. Peripheral diabetic neuropathy: current concepts in treatment. Ann Pharmacother 1995; 29:769-77). Originally, the major mechanism of the analgesic effect of tricyclic antidepressants was believed to be related to serotonin reuptake inhibition. Animal models of peripheral neuropathic pain have shown that tricyclic antidepressants act as sodium channel blockers, similar to local anesthetic and antiarrhythmic agents (Hunter J C, Gogas K R, Hedley L R, Jacobson L O, Kassotakis L, Thompson J, and Fontana D J (1997) The effect of novel anti-epileptic drugs in rat experimental models of acute and chronic pain. Eur J Pharmacol 324: 153-160).

Amitriptyline drug is effective in the treatment of postherpetic neuralgia, diabetic neuropathy, and other neuropathic pain syndromes. Oral amitriptyline achieves a good or moderate response in about two-thirds of patients with postherpetic neuralgia and three-quarters of patients with painful diabetic neuropathy; such neurogenic pain syndromes are often unresponsive to narcotic analgesics (Bryson H M and Wilde M I (1996) Amitriptyline. A review of its pharmacological properties and therapeutic use in chronic pain states. Drugs Aging 8: 459-476). Whether analgesic effects of amitriptyline are linked to its mood-altering activity and/or are attributable to a discrete pharmacological action is unknown. Above the therapeutic plasma concentration of 0.3 to 0.8 μM, the tricyclic antidepressants have significant effects on the cardiovascular system, including direct depression of the myocardium and evidence of prolonged conduction times (Nattel S (1985) Frequency-dependent effects of amitriptyline on ventricular conduction and cardiac rhythm in dogs. Circulation 72: 898-906); with an overdose of >3 μM, these effects may be life-threatening. The known physiological targets of tricyclic antidepressants in the central nervous system are the 5-HT₂ serotonin receptors and the α₁-adrenergic receptors.

In addition to these primary targets, tricyclic antidepressants are also effective K⁺ and Na⁺ channel blockers. For example, tricyclic imipramine inhibits transient K⁺ channels in hippocampal neurons with an IC₅₀ of ˜6 μM (Kuo C C (1998) Imipramine inhibition of transient K⁺ current: An external open channel blocker preventing fast inactivation. Biophys J 12: 2845-2857).

Another anti-depressant milnacipran and methods for its synthesis are described in U.S. Pat. No. 4,478,836. Milnacipran (FIG. 6; midalcipran, midacipran, F 2207) inhibits the uptake of both, norepinephrine (NE) and serotonin (5-HT), with an NE to 5-HT ratio of 2:1 (Moret C, et al. Biochemical profile of midalcipran (F 2207), 1-phenyl-1-diethyl-aminocarbonyl-2-aminomethyl-cyclopropane (Z) hydrochloride, a potential fourth generation antidepressant drug. Neuropharmacology 1985:24 (12): 1211-9) but does not affect the uptake of dopamine. Milnacipran has no affinity for α or β adrenergic, muscarinic, histaminergic, and dopaminergic receptors. This suggests that milnacipran has a low potential to produce anticholinergic, sedative, and stimulant effects. Milnacipran does not affect the number of beta adrenoceptors in rat cortex after chronic administration. Additional information regarding milnacipran may be found in the Merck Index, 12th Edition, at entry 6281.

Milnacipran (Ixel©, Pierre Fabre), has demonstrated numerous adverse reactions in human clinical trials with tolerability decreasing with increasing dose (Puech A. et al., 1997, Int. Clin. Psychopharm., 12:99-108). In the double-blind, randomized, multicenter clinical study the most frequent spontaneously reported adverse events for 100 mg/day milnacipran twice daily were as follows: abdominal pain (13%), constipation (10%), and headache (9%). The incidence of certain adverse events increases with dosage, including nausea, vomiting, sweating, hot flashes, palpitations, tremor, anxiety, dysuria, and insomnia.

It is important to note that in one of the early depression trials, even after one week of milnacipran dose escalation employed to reduce side effects, the most commonly reported reason for discontinuation of treatment because of adverse effects was nausea and vomiting (Leinonen E., Acta Psychiatr. Scand., 1997; 96:497-504). In the recent fibromyalgia clinical trial with the long dose escalation period (four weeks) which was implemented in order to reduce milnacipran side effects and increase patient's tolerance, the most common dose-related side effect reported by patients was nausea (Cypress Bioscience Inc., Cypress Bioscience Inc. Announces Final Results of Milnacipran Phase II Clinical Trial in Fibromyalgia, Media Release, Mar. 21, 2003).

The currently immediate available release formulation of milnacipran is not suitable for the treatment of health conditions that require milnacipran doses equal or above 100 mg/day given either as once a day or twice a day due to high incidence of treatment-emergent side effects that leads to poor patient's tolerance. Higher doses are required in the treatment of severe depression and other associated disorders. As shown in one of the early antidepressant clinical trials, milnacipran dosage of 200 mg/day was superior to the lower doses (Von Frenckell R et al., 1990, Int. Clin. Psychopharmacology, 5:49-56). Milnacipran dosing regime of 100-250 mg daily was recently reported for the treatment of fibromyalgia (U.S. Pat. No. 6,602,911). It would be very difficult to reach the upper limits of the dose range using the currently available formulation due to the dose related treatment emergent side effects and the need to titrate over a long period to reach the required dose.

U.S. Pat. No. 5,578,645 teaches the method for treating acute or chronic pain in a mammal comprising the administration of a therapeutically effective amount of an analgesic solution composed of at least one branched chain amino acid selected from the group consisting of leucine, isoleucine, and valine, or administering a therapeutically effective amount of an analgesic solution comprising an analgesic agent selected from the group consisting of an opiate, an agonistic-antagonistic agent, and an anti-inflammatory agent, and at least one branched chain amino acid selected from the group consisting of leucine, isoleucine, and valine.

U.S. Pat. No. 4,769,372 describes a method for treating chronic pain or chronic cough in a patient while preventing or alleviating the development of constipation or other symptoms of intestinal hypomotility wherein an opiate analgesic or antitussive such as morphine, meperidine, oxycodone, hydromorphone, codeine and hydrocodone is administered to the patient together with an opiate antagonist such as naloxone, naloxone glucuronide or nalmefene glucuronide. However successful this therapeutic combination may be in inhibiting the development of constipation or other symptoms of intestinal hypomotility, it does not address the problems of tolerance and/or dependence that are associated with the long term administration of narcotic analgesics.

U.S. Pat. No. 6,054,451 discloses the analgesic composition comprising (R) or (S)-5-(2-azetidinylmethoxy)-2-chloropyridine, or their salts; and an analgesic-potentiating amount of at least one nontoxic N-methyl-D-aspartate receptor antagonist for alleviating pain e.g. arthritic, lumbosacral or musculo-skeletal pain or pain associated with a sore throat. It has been claimed that reduced dosages of analgesic are required. U.S. Pat. No. 6,007,841 discloses analgesic composition comprises at least one narcotic agonist-antagonist analgesic and a narcotic agonist-antagonist analgesic-potentiating amount of at least one N-methyl-D-aspartate receptor antagonist.

U.S. Pat. No. 5,516,803 discloses a composition comprising a tramadol material and a nonsteroidal antiinflammatory drug, and its use. The compositions are pharmacologically useful in treating pain and tussive conditions. The compositions are also subject to less opioid side-effects such as abuse liability, tolerance, constipation and respiratory depression. Furthermore, where the components of the compositions are within certain ratios the pharmacological effects of the compositions are superadditive (synergistic). U.S. Pat. No. 5,336,691 discloses a composition comprising a tramadol material and acetaminophen, and its use. As used herein tramadol refers to various forms of tramadol. The compositions are pharmacologically useful in treating pain and tussive conditions. The compositions are also subject to less opioid side-effects such as abuse liability, tolerance, constipation and respiratory depression. Furthermore, where the components of the compositions are within certain ratios the pharmacological effects of the compositions are superadditive (synergistic).

U.S. Pat. No. 5,919,826 discloses the analgesic effectiveness of tramadol significantly enhanced by administering tramadol with the administration of an analgesia-enhancer which is a nontoxic NMDA receptor blocker and/or a nontoxic substance that blocks at least one major intracellular consequence of NMDA receptor activation for treating arthritis.

U.S. Pat. Nos. 4,656,177 and 4,777,174 disclose combinations of non-narcotic analgesics/nonsteroidal anti-inflammatory drugs and/or narcotic analgesics and caffeine. The compositions elicit a more potent and more rapid analgesic response than if the pain reliever is given alone.

U.S. Pat. No. 5,248,678 teaches a method of increasing the arousal and alertness of comatose patients or nea-comatose patients comprising administering to the patients effective amounts of an adenosine receptor antagonist, such as caffeine, and a GABA agonist, such as gabapentin.

U.S. Pat. No. 6,326,374 discloses compositions that comprise a GABA analog, such as gabapentin or pregabalin in combination with caffeine for the treatment of pain in mammals.

U.S. Pat. No. 6,001,876 discloses a method of using certain analogs of glutamic acid and gamma-aminobutyric acid in pain therapy

U.S. Pat. No. 6,187,338 discloses composition containing (a) neuropathic pain-alleviating amount of at least one anticonvulsant, (b) an anticonvulsant-potentiating amount of at least one nontoxic antagonist for the NMDA receptor or nontoxic substance that blocks a major intracellular consequence of NMDA receptor activation, and a therapeutically effective amount of at (c) least one analgesic. The analgesic is at least one member selected from the group consisting of acetaminophen, aspirin, diclofenac, diflusinal, etodolac, fenbufen, fenoprofen, flufenisal, flurbiprofen, ibuprofen, indomethacin, ketoprofen, ketoro ac, meclofenamic acid, mefenamic acid, nabumetone, naproxen, oxaprozin, phenylbutazone, piroxicam, sulindac, tolmetin and zomepirac.

U.S. Pat. No. 6,242,488 discloses a method for preventing or treating pain in a mammal comprising administering an effective amount of a composition comprising a GABA analog and a non-steroidal anti-inflammatory drug together with a pharmaceutically acceptable excipient, carrier, or diluent thereof.

U.S. Pat. No. 6,242,488 discloses GABA analogs that are useful to prevent and treat gastrointestinal damage and ethanol withdrawal syndrome. Preferred treatments employ gabapentin or pregabalin. Typical compounds include (1-aminomethyl-3-methylcyclohexyl)acetic acid, (1-aminomethyl-3-methylcyclopentyl)acetic acid, (S)-3-(aminomethyl)-5-methylhexanoic acid, 3-aminomethyl-5-methyl-hexanoic acid. and (1-aminomethyl-3,4-dimethylcyclopentyl)acetic acid.

U.S. Pat. No. 6,406,716 discloses the effectiveness of an anticonvulsant such as gabapentin for alleviating neuropathic pain which is potentiated by a nontoxic antagonist for the N-methyl-D-aspartate receptor or nontoxic substance that blocks a major intracellular consequence of N-methyl-D-aspartate receptor activation.

U.S. Pat. No. 6,835,398 discloses a method of treating patients, particularly for pain associated with diseases including erythromelalgia, chronic regional pain syndrome, and reflex sympathetic dystrophy, which involves orally administering high doses of magnesium. The magnesium is introduced through several daily administrations, totaling approximately 2-12 times the RDA for magnesium (600 mg to 5 gm elemental magnesium). These higher levels are achieved through increasing daily dosage amounts gradually in response to patient tolerance and using a more well-tolerated form of magnesium preferably a magnesium solution. Total magnesium intake is divided over several doses per day and taken with copious amounts of water.

U.S. Pat. No. 6,417,184 discloses a triple drug therapy, pharmaceutical kit, composition, and method of treatment regimen utilized as a combination of effective amounts of an anxiolytic agent, centrally acting alpha anti-adrenergic agent, and central nervous system stimulant for the reduction or prevention of dizziness, drowsiness, depression, delirium, lethargy, mania, orthostatic hypotension, restlessness, weakness in the extremities, and difficulty in being mobile negative side effects caused by therapeutic agents utilized in the treatment of acute and chronic pain syndromes.

US Pat. Application No. 20070087977 discloses a pharmaceutical composition comprising an analgesic agent, a blood brain barrier (BBB) transport protein activator and a pharmaceutically acceptable excipient, wherein the analgesic agent is present in an amount sufficient to produce an analgesic effect, and wherein the BBB transport protein activator is present in an amount sufficient to reduce a central nervous system (CNS) effect of the analgesic agent.

US Pat. Application No. 20070042969 discloses a method for treating pain in painful diabetic neuropathy which comprises administering in combination a first agent that comprises a compound as defined, illustratively lacosamide, and a second agent effective to provide enhanced treatment of pain, by comparison with the first agent alone. The second agent illustratively comprises an analgesic, an anticonvulsant, an antidepressant or an NMDA receptor antagonist.

US Pat. Application No. 20060264509 discloses a method for using α₂δ subunit calcium channel modulators or other compounds that interact with the α₂δ calcium channel subunit in combination with one or more compounds with smooth muscle modulatory effects to treat pain. According to application, α₂δ subunit calcium channel modulators include GABA analogs (e.g., gabapentin and pregabalin), fused bicyclic or tricyclic amino acid analogs of gabapentin, and amino acid compounds. Compounds with smooth muscle modulatory effects include antimuscarinics, β3 adrenergic agonists, spasmolytics, neurokinin receptor antagonists, bradykinin receptor antagonists, and nitric oxide donors.

US Pat. Application No. 20060159743 provides a method of treating a patient suffering from a pain state by administering to the patient a gastric retentive dosage form of gabapentin that is capable of administration in once-daily or twice daily dosing regimens. By reducing the need to administer gabapentin from the thrice-daily administrations characteristic of immediate release gabapentin, the gastric retentive gabapentin dosage forms provided herein have the advantages of improving patient compliance for gabapentin treatment. In addition to the foregoing, the gastric retentive gabapentin dosages forms also exhibit decreased blood plasma concentrations and increased bioavailability throughout the dosing regimen.

US Pat. Application No. 20050009916 discloses a treatment for central neuropathic pain with an analgesic composition that consists essentially of an N-methyl-D-aspartate (NMDA) receptor antagonist. In one embodiment, the application includes chronic administration of the (NMDA) receptor antagonist. In another embodiment, the application is the use of an NMDA receptor antagonist or component thereof for the manufacture of a medicament than includes an analgesic component that consists essentially of an NMDA receptor antagonist for the chronic treatment of central neuropathic pain.

US Pat. Application No. 20060009478 discloses methods and materials, including novel compositions, dosage forms and methods of administration, useful for treating back pain using opioid antagonists, including combinations of opioid antagonists and opioid agonists. Methods and materials comprising opioid antagonists or combinations opioid antagonists and agonists may optionally include one or more additional therapeutic agents.

US Pat. Application No. 20050209319 discloses methods and compositions for treating or preventing local pain or discomfort, particularly local neuropathic pain via topical application directly to skin or mucosal tissue at the site of pain or discomfort are disclosed. Compositions comprising prodrugs of gamma amino butyric acid analogs, such as prodrugs of gabapentin or pregabalin, and optionally a topical anesthetic agent are also disclosed.

US Pat. Application No. 20010008889 discloses the analgesic effectiveness of tramadol is significantly enhanced by administering tramadol prior to, with or following the administration of an analgesia enhancer which is a nontoxic NMDA receptor blocker and/or a nontoxic substance that blocks at least one major intracellular consequence of NMDA receptor activation.

US Pat. Application No. 20010036943 discloses pharmaceutical compositions for the treatment of acute, chronic and/or neuropathic pain. The pharmaceutical compositions are comprised of a therapeutically effective combination of a nicotine receptor partial agonist and an analgesic agent and a pharmaceutically acceptable carrier. The analgesic agent is selected from opioid analgesics, NMDA antagonists, substance P antagonists, COX 1 and COX-2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, and botulinum toxin. The method of using these compounds and a method of treating acute, chronic and/or neuropathic pain and migraine in a mammal including a human is also disclosed.

US Pat. Application No. 20020058656 discloses a triple drug therapy, pharmaceutical kit, composition, and method of treatment regimen utilized as a combination of effective amounts of an anxiolytic agent, centrally acting alpha anti-adrenergic agent, and central nervous system stimulant for the reduction or prevention of dizziness, drowsiness, depression, delirium, lethargy, mania, orthostatic hypotension, restlessness, weakness in the extremities, and difficulty in being mobile negative side effects caused by therapeutic agents utilized in the treatment of acute and chronic pain syndromes.

US Pat. Application No. 20030133951 discloses pharmaceutical compositions for the treatment of acute, chronic and/or neuropathic pain. The pharmaceutical compositions are comprised of a therapeutically effective combination of a nicotine receptor partial agonist and an analgesic agent and a pharmaceutically acceptable carrier. The analgesic agent is selected from opioid analgesics, NMDA antagonists, substance P antagonists, COX-1 and COX-2 inhibitors, tricyclic antidepressants (TCA), selective serotonin reuptake inhibitors (SSRI), capsaicin receptor agonists, anesthetic agents, benzodiazepines, skeletal muscle relaxants, migraine therapeutic agents, anti-convulsants, anti-hypertensives, anti-arrythmics, antihistamines, steroids, caffeine, and botulinum toxin. The method of using these compounds and a method of treating acute, chronic and/or neuropathic pain and migraine in a mammal including a human is also disclosed.

US Pat. Application No. 20040002543 discloses composition and method for treating sinus headache or sinus pains including analogs of glutamic acid and gamma-aminobutyric acid in combination with a decongestant.

US Pat. Application No. 20030232787 discloses a novel combination effective for alleviating pain comprising a pain alleviating effective amount of an endothelin receptor antagonist or a pharmaceutically acceptable salt thereof and from 1 to 3 compounds independently selected from the group consisting of antiepileptic compounds having pain alleviating properties and analgesics, and pharmaceutically acceptable salts thereof, and pharmaceutical compositions comprising same. The administration of endothelin receptor antagonists in these novel combinations results in an improved reduction in the frequency and severity of pain. The incidence of unwanted side effects can be reduced by these novel combinations in comparison to using higher doses of a single agent treatment to achieve a similar therapeutic effect. The present invention is also directed to methods of using effective amounts of the novel combinations and pharmaceutical compositions thereof to treat pain in mammals, including a human.

US Pat. Application No. 20060241134 relates to the combination of certain active compounds from the acid pump antagonist class and compounds which modify gastrointestinal motility.

US Pat. Application No. 20060240128 pertains to an analgesic composition comprising an analgesic drug in an extended release form in combination with an analgesia-enhancing amount of a nontoxic N-methyl-D-aspartate receptor antagonist in an immediate release form.

US Pat. Application No. 20060178354 relates to the treatment of chronic pain using DHEA or derivatives thereof either alone or in combination with at least one other drug. The application also includes compositions comprising DHEA or a derivative thereof and a second drug.

US Pat. Application No. 20050282859 discloses compositions and methods for treatment of genitourinary disorders (e.g., urge incontinence). The compositions may generally include a dual-acting SNRI-NMDA antagonist (e.g., bicifadine and/or milnacipran). Alternatively, the compositions may generally include an SNRI and an NMDA antagonist.

US Pat. Application No. 20050245460 relates to methods and compositions for treating CNS-related disorders. A pharmaceutical composition comprising: (a) an NMDA receptor antagonist; (b) a second agent, wherein said agent is an anti-epileptic drug (AED); and (c) a pharmaceutically acceptable carrier, wherein at least one of said NMDA receptor antagonist or said second agent is provided in an extended release dosage form.

US Pat. Application No. 20050203142 and 20050119194 disclose methods of treating, preventing, modifying and managing various types of pain. Specific methods comprise the administration of an immunomodulatory compound, or a pharmaceutically acceptable salt, solvate, hydrate, stereoisomer, clathrate, or prodrug thereof, alone or in combination with a second active agent and/or surgery, psychological or physical therapy. Pharmaceutical compositions, single unit dosage forms, and kits suitable for use in methods of the invention are also disclosed.

US Pat. Application No. 20050065176 relates to a combination of an α₂δ ligand and an AChE inhibitor for use in therapy, particularly in the treatment of pain, particularly neuropathic pain. Particularly preferred α₂δ ligands are gabapentin and pregabalin. Particularly preferred ACHE inhibitors are donepezil (Aricept™), tacrine (Cognex™), rivastigmine (Exelon™) physostgmine (Synapton™), galantamine (Reminyl), metrifonate (Promem), neostigmine (Prostigmin) and icopezil.

US Pat. Application No. 20050059715 relates to a combination, particularly a synergistic combination, of an α₂δ ligand and a dual serotonin-noradrenaline re-uptake inhibitor (DSNRI) or one or both of a selective serotonin re-uptake inhibitor (SSRI) and a selective noradrenaline re-uptake inhibitor (SNRI), and pharmaceutically acceptable salts thereof, pharmaceutical compositions thereof and their use in the treatment of pain, particularly neuropathic pain.

US Pat. Application No. 20040092522 relates to a combination of an α₂δ ligand and a PDE-V inhibitor for use in therapy, particularly in the curative, prophylactic or palliative treatment of pain, particularly neuropathic pain. Particularly preferred α₂δ ligands are gabapentin and pregabalin. Particularly preferred PDE-V inhibitors are sildenafil, vardenafil and tadalafil.

WO/2005/102390 application relates to a synergistic combination of an α2δ ligand and an NMDA receptor antagonist (NMDA antagonist), suitably having affinity for the NR2B-subtype (NR2B antagonist), useful for the treatment of pain. It also relates to a method for treating pain through the use of effective amounts of synergistic combinations of an α2δ ligand and an NMDA antagonist.

US Pat. Application No. 20040063751 discloses a method of treating, preventing, or inhibiting ALS, in a subject in need of such treatment, inhibition or prevention. The method comprises administering to a subject one or more cyclooxygenase-2 selective inhibitor(s) or isomer(s) or pharmaceutically acceptable salt(s), ester(s), or prodrug(s) thereof, in combination with one or more second drugs, wherein the amount of the cyclooxygenase-2 selective inhibitor(s) or isomer(s) or pharmaceutically acceptable salt(s), ester(s), or prodrug(s) thereof in combination with the amount of second drug(s) constitutes an ALS treatment, inhibition or prevention effective amount.

US Pat. Application No. 20030176505 is directed to novel combinations of anti-epileptic compounds that demonstrate pain alleviating properties, with compounds selected from the group consisting of analgesics, NMDA receptor antagonists, and NSAIDs and pharmaceutical compositions comprising same. It has been discovered that the administration of anti-epileptic compounds that demonstrate pain alleviating properties in these novel combinations results in an improved reduction in the frequency and severity of pain. It is also believed that the incidence of unwanted side effects can be reduced by these novel combinations in comparison to using higher doses of a single agent treatment to achieve a similar therapeutic effect. It is also directed to methods of using effective amounts of the novel pharmaceutical compositions to treat pain in mammals.

US Pat. Application No. 20020115705 is directed to novel combinations of anti-epileptic compounds that demonstrate pain alleviating properties, with compounds selected from the group consisting of analgesics, NMDA receptor antagonists, and NSAIDs and pharmaceutical compositions comprising same. It has been discovered that the administration of anti-epileptic compounds that demonstrate pain alleviating properties in these novel combinations results in an improved reduction in the frequency and severity of pain. It is also believed that the incidence of unwanted side effects can be reduced by these novel combinations in comparison to using higher doses of a single agent treatment to achieve a similar therapeutic effect. It is also directed to methods of using effective amounts of the novel pharmaceutical compositions to treat pain in mammals.

U.S. Pat. No. 6,593,368 and U.S. Pat. No. 6,942,876 disclose novel combinations of anti-epileptic compounds that demonstrate pain alleviating properties, with compounds selected from the group consisting of analgesics, N-methyl-D aspartate (NMDA) receptor antagonists and non-steroidal anti-inflammatory drugs (NSAIDS) and pharmaceutical compositions comprising same. Specifically, the patents disclose a combination of an effective amount of an anti-epileptic compound having pain alleviating properties and a compound which is a NMDA receptor antagonist and another combination, comprising a synergistic amounts of gabapentin and celecoxib in a weight/weight ratio of from 1:50 to 50:1, respectively.

US Pat. Application No. 20050038062 discloses methods and compositions for treating subjects with pain, including neuropathic pain, using opioid antagonists or combinations of opioid antagonists and opioid agonists, including, for example, the amount of an opioid antagonist enhances the neuropathic pain-alleviating potency of an opioid agonist. The composition of opioid antagonists or combinations of opioid antagonists and opioid agonists further comprises a local anesthetic that is bupivicaine hydrochloride, chloroprocaine hydrochloride, dibucaine, dibucaine hydrochloride, etidocaine hydrochloride, lidocaine, lidocaine hydrochloride, mepivacaine hydrochloride, piperocaine hydrochloride, prilocaine hydrochloride, procaine hydrochloride, propoxycaine hydrochloride tetracaine, or tetracaine hydrochloride.

US Pat. Application No. 20060240128 and WO application 2004022002 disclose an analgesic composition comprising an analgesic drug in an extended release form in combination with an analgesia-enhancing amount of a nontoxic N-methyl-D-aspartate receptor antagonist in an immediate release form. The nontoxic NMDA receptor antagonist is at least one member selected from the group consisting of dextromethorphan, dextrorphan, memantine, amantidine, d-methadone and their pharmaceutically acceptable salts; or the nontoxic NMDA receptor antagonist is present in an immediate release carrier; or the analgesic drug is selected from the group consisting essentially of non-narcotic analgesics, coal tar analgesics, nonsteroidal anti-inflammatory drugs, gabapentin, sub stance P antagonists, cap saicin, cap saicinoids, and cyclooxygenase-II (COX II) inhibitors; or the weight ratio of the analgesic drug to the nontoxic NMDA receptor antagonist ranges from about 2:1 to about 1:10; or the weight ratio of the analgesic drug to the nontoxic NMDA receptor antagonist ranges from about 1:1 to about 1:5. The analgesic composition wherein the analgesic drug is an analgesically effective amount of at least one opioid analgesic and the analgesic composition is substantially free of opioid antagonist. The opioid analgesic is at least one member selected from the group consisting of alfentanil, allylprodine, alphaprodine, anileridine, benzylmorphine, b ezitrami de, buprenorphine, butorphanol, clonitazine, codeine, desomorphine, dextromoramide, dezocine, di ampromide, diamorphone, dihydrocodeine, dihydromorphine, dimenoxadol, dimepheptanol, dimethylthiambutene, dioxaphetyl butyrate, dipipanone, eptazocine, ethoheptazine, ethymethylthiambutene, ethylmorphine, etonitazene, fentanyl, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papaveretum, pentazocine, phenadoxne, phenomorphan, phenazocine, phenoperidine, piminodine, piritramide, propheptazine, promedol, properidine, propoxyphene, sufentanyl, tilidine, tramadol and their pharmaceutically acceptable salts.

US Pat. Application No. 20060167032 discloses the treatment of disorders of the ventral nervous system (CNS) by the administration of a GABA analog such as gabapentin or pregablin, an NMDA receptor antagonist such as dextromethorphan or d-methodone and, optionally, another pharmacologically active substance, e.g., one which is effective for the treatment of a CNS disorder. The pharmaceutical composition contains a therapeutically effective amount of at least one other pharmaceutically active substance (c) which is a drug or drug combination for the treatment of a CNS disorder selected from the group consisting of nicotine, nicotinic compounds, tacrine, donezepil, carbidopa in combination with levodopa, selegiline, bromocriptine, haloperidol, clonidine, pimozide, fluphenazine, benzodiazepines, clonazepam, clorpromazine, fluoxetine, clomipramine, amitriptyline, nortriptyline, imipramine, buspirone, bupropion hydrochloride, venlafaxine, milnacipran, duloxetine, mirtazapine, nefazodone, paroxetine, sertraline, riluzole, trazodone, doxepin and methylphenidate. The CNS disorder is presenile dementia, senile dementia, movement disorder, hyperkinesias, mania, attention deficit disorder, depression, anxiety, obsessive-compulsive disorder, dyslexia, schizophrenia, headache disorder, epilepsy, Tourette's syndrome or Asperger's syndrome.

WO application 2004/089343 discloses water-soluble tablets that dissolve to form clear aqueous solutions, and processes for their preparation. The process includes compressing a mixture of (a) at least one water-soluble active ingredient; (b) one or more water soluble sugar alcohols; (c) one or more water-soluble lubricants; and (d) one or more pH modifiers. The tablet dissolves in about 3 minutes in about 30 ml of water to give a clear solution. The one or more water-soluble active ingredients may be metfonnin hydrochloride, gabapentin, glibenclamide, glipizide, diltiazem hydrochloride, verapamil hydrochloride, bupropion hydrochloride, propranolol hydrochloride, dextromethorphan hydrobromide, diphenhydramine hydrochloride, disopyramide hydrochloride, tramadol, fluoxetine hydrochloride, paroxetine hydrochloride, pentoxifylline hydrochloride, and diclofenac sodium. The one or more water soluble sugar alcohols may be one or more of sorbitol, mannitol, spray dried mannitol, xylitol, erythritol isomalt and hydrogenated starch hydrolysates and combinations thereof. The one or more water-soluble lubricants may be one or more of DL-leucine, sodium lauryl sulphate, magnesium lauryl sulphate and polyethylene glycol. The one or more pH modifiers may be one or more of potassium hydroxide, sodium hydroxide, monosodium citrate, citric acid and the like. While the patent application claims the process for water soluble tablets are novel, U.S. Pat. Nos. 4,347,235 and 3,976,601 discloses such process for making such water soluble tablets. US Pat. Application No. 20060240128 discloses a combined analgesic composition having at least one analgesic drug in an extended release form and at least one nontoxic N-methyl-D-aspartate receptor antagonist in an immediate release form, where the activity of the analgesic drug is enhanced by the at least one nontoxic N-methyl-D-aspartate receptor antagonist. Preferably, the analgesic drug is an opioid analgesic, the at least one nontoxic N-methyl-D-aspartate receptor antagonist is dextromethorphan, and the analgesic composition is substantially free of opioid antagonist.

US Pat. Application No. 20020035105 describes the neuropathic pain alleviating effectiveness of an antidepressant is significantly potentiated by administering the antidepressant prior to, with or following the administration of a nontoxic NMDA receptor antagonist.

U.S. Pat. No. 7,244,767 relates to the use of N-acylvanillinamide derivatives capable of activating the peripheral receptor CB1 of cannabinoids. In particular, the present invention relates to the use of compounds for the preparation of a medicinal product which is capable of activating the peripheral receptor CB1 of cannabinoids.

Various treatments and/or methods suggested for the treatment of dysmenorrhea and/or endometriosis include Amidinoureas (U.S. Pat. No. 4,241,087); combination of aspirin or ibuprofen and a diuretic (U.S. Pat. No. 4,888,343); .beta.-adrenergic agonist (U.S. Pat. No. 6,126,959); histidine (U.S. Pat. No. 6,207,696); noninvasive electrical stimulation of a single acupuncture site (U.S. Pat. No. 6,718,202); proanthocyanidins (U.S. Pat. No. 6,372,266); thromboxane A₂ receptor antagonist such as ifetroban, alone or in combination with a non-steroidal anti-inflammatory drug (U.S. Pat. No. 5,605,917); a progestational hormone (U.S. Pat. No. 4,016,270); modulating antileukoprotease activity (U.S. Pat. No. 6,544,740); danazol (U.S. Pat. No. 4,997,653); enzothiophenes (U.S. Pat. No. 5,827,844); composition containing activated chlorine dioxide and phosphates (U.S. Pat. No. 5,935,592); Anti-VEGF agents (U.S. Pat. No. 6,121,230); low doses of an estrogen agent and a progestin agent (U.S. Pat. No. 6,265,393); Bisphosphonates (U.S. Pat. No. 8,257,742); interleukin-5 antagonist (U.S. Pat. No. 8,192,736); a combination of an aromatase inhibitor, a progestin and an oestrogen (U.S. Pat. No. 7,910,570); short term induction treatment with an LH-RH antagonist (U.S. Pat. No. 7,666,836); antiallergic agent (U.S. Pat. No. 7,585,854); composition comprising the phytochemical Diindolylmethane (DIM), as well as its precursor, Indole-3-carbinol (I3C), and cogener, 2-(Indol-3-ylmethyl)-3,3′diindolylmethane (LTR-1) (U.S. Pat. No. 7,384,972); Tetrahydroquinoline derivatives (U.S. Pat. No. 6,962,928); Tumor necrosis factor antagonists (U.S. Pat. No. 6,663,865); ethinylestradiol and drospirenone (U. S. Patent Application No. 20060128679); alpha-adrenergic blockers (U. S. Patent Application No. 20060128719); an antagonist of a prostaglandin EP2 and/or EP4 receptor (U. S. Patent Application No. 20080206309); Trandermal NSAIDs (U. S. Patent Application No. 20090291140); tranilast (U. S. Patent Application No. 20120157535); Oxytocin Receptor Antagonists (U. S. Patent Application No. 20080242666); N-Acetyl-L-Cysteine (U.S. Patent Application No. 20120238627); quinagolide (U. S. Patent Application No. 20120157489); mifepristone (U. S. Patent Application No. 20110208118); Eicosapentaenoic acid and/or docosahexahenoic acid (U. S. Patent Application No. 20110054030); progesterone receptor antagonist (U. S. Patent Application No. 20110046098); a progestin and an oestrogen (U. S. Patent Application No. 20110009373); mineral corticoid receptor antagonists (U. S. Patent Application No. 20110003778); Prostglandin E2 Receptors Inhibitors (U. S. Patent Application No. 20100249125); IKK Inhibitors (U. S. Patent Application No. 20100069387); A Combination of a Gestagen and (6s)-5-Methyltetrahydrofolate (U. S. Patent Application No. 20090060997); Vitamin D Compounds (U. S. Patent Application No. 20080280860); Alkyl substituted Pyridines (U. S. Patent Application No. 20080182830); Piperazine urea derivatives (U. S. Patent Application No. 20080119471); prokineticin-1 inhibitors (U. S. Patent Application No. 20060287258); human chorionic gonadotropin (20060258568); COX-2 inhibitors (U. S. Patent Application No. 20060173062); Anti-inflammatory composition (U. S. Patent Application No. 20050220912); and an aryl hydrocarbon receptor binding ligand (U. S. Patent Application No. 20020147155).

The principal inventor has disclosed in U.S. Patent Application No. 20110039875 methods and compositions for the treatment of neuropathic pain. In certain embodiments of that invention, compositions comprising, dextromethorphan (or other N-methyl-D-aspartate receptor antagonist), tramadol, and gabapentin can synergistically act to reduce pain in a human patient. Pharmaceutical compositions may also comprise a capsaicinoid, an esterified capsaicinoid, and/or a tricyclic antidepressant.

Now, the inventors have surprisingly found in a highly unexpected fashion, that a combination of a non-toxic NMDA receptor antagonist such as dextromethorphan with tramadol or its analog and an anticonvulsant and/or a tricyclic anti-depressant exhibits significant palliative effects on the pain associated with primary and secondary dysmenorrhea.

Accordingly, an object of the invention is to provide methods and compositions for the treatment of pain associated with primary and secondary dysmenorrhea which provide effective control of pain without the harmful side effects associated with traditional analgesics, such as respiratory depression, disturbed sleep patterns, diminished appetite, seizures, and psychological and/or physical dependency.

In addition, the present invention can avoid the liability of gastrointestinal and liver toxicity by omitting acetaminophen, aspirin and other NSAID's. Acetaminophen toxicity is well known and represents a significant drawback of all formulations that contain it. The limiting dose of acetaminophen is on the order of 2 grams per day. It has also been determined that intentional overdose of acetaminophen is the second most common method of committing suicide in Europe. Thus, reducing or eliminating exposure to acetaminophen is of significant importance.

These and other objects and features of the invention will be apparent from the following description.

### SUMMARY OF THE INVENTION

It is an object of the present invention to provide a method and pharmaceutical formulation, (medicament), which allows for reduced plasma concentrations of active ingredients, while still providing effective pain management for primary and secondary dysmenorrhea.

It is a further object of the present invention to provide a method and a pharmaceutical formulation (medicament) for effectively treating patients in pain associated with primary and secondary dysmenorrhea. Accordingly, the present invention provides a method that comprises administering a pharmaceutical composition comprising an analgesic combination that includes a NMDA receptor antagonist or a pharmaceutically acceptable salt thereof, an anticonvulsant and/or a tricyclic anti-depressant or a pharmaceutically acceptable salt thereof, and tramadol or its analog, or a pharmaceutically acceptable salt thereof. By this method is achieved an analgesic preparation which produces prolonged and effective pain management, while at the same time exhibits reduced side effects and decreases the liability to dependence and tolerance which the patients may experience when subjected to prolonged treatment with an opiate.

In accordance with the present invention, a NMDA receptor antagonist can be dextromethorphan, magnesium, dextrorphan, ketamine, amantadine, memantine, eliprodil, ifenprodil, phencyclidine, MK-801, dizocilpine, CCPene, flupirtine, or derivatives or salts thereof. Even though magnesium exerts various physiological effects, for the purpose of teaching the present invention, it will be simply referred as NMDA receptor antagonist.

An anticonvulsant can be, for example, gabapentin, pregabalin, 3-methyl gabapentin or derivatives thereof.

A tramadol or its analog can be any one of (1R,2R or 1S,2S)-(dimethylaminomethyl)-1-(3-methoxyphenyl)-cyclohexanol (tramadol), its N-oxide derivative (“tramadol N-oxide”), its O-desmethyl derivative (“O-de smethyl tramadol”), venlafaxine, (R/S)-1-[2-(dimethylamino)-1-(4-methoxyphenyl)ethyl]cyclohexanol and O-desmethylvenlafaxine or mixtures, stereoisomers or racemates thereof.

The present invention further provides a method and composition for effectively treating patients in pain associated with primary and secondary dysmenorrhea which avoids the toxicities associated with NSAID or acetaminophen therapy. The method comprises administering a pharmaceutical composition to a patient in need of treatment for pain, wherein the pharmaceutical composition comprises an analgesic combination comprising a NMDA antagonist or a pharmaceutically acceptable salt thereof, an anticonvulsant and/or a tricyclic anti-depressant or a pharmaceutically acceptable salt thereof, and tramadol or its analog, or a pharmaceutically acceptable salt thereof. In accordance with the present invention, the composition can be essentially free of a NSAID or acetaminophen. Particularly relevant NSAIDs include ibuprofen, diclofenac, diflunisal, etodolac, fenoprofen, flurbiprofen, indomethacin, ketoprofen, ketorolac, mefenamic acid, meclofenamate, nabumetone, naproxen, oxaprozin or piroxicam. If the patient is separately administered a NSAID and/or acetaminophen, the amount administered is not enough to induce one or more toxicities associated with the use of the NSAID and/or acetaminophen.

Although tramadol/acetaminophen formulations containing a slew of other pharmaceutically active agents such as decongestants, antitussives, antihistamines or suspected adjuvants have been suggested in a general fashion, the particular combination of NMDA receptor antagonist, tramadol or its analog and anticonvulsant and/or a tricyclic anti-depressant has not been previously recognized or appreciated for the treatment of pain associated with primary and secondary dysmenorrhea. Similarly, the particular combination of NMDA receptor antagonist, tramadol or its analog and anticonvulsant and/or a tricyclic anti-depressant in a composition essentially free of a NSAID and/or acetaminophen has not been recognized or appreciated.

In accordance with the present invention, the ratio of NMDA antagonist to tramadol or its analog can be from about 15:1 to 1:15, about 10:1 to 1:10, about 5:1 to 1:5, or about 1:2 to 1:2. The ratio of NMDA antagonist to anticonvulsant and/or a tricyclic anti-depressant to tramadol or its analog can be from about 90:1:1 to 1:90:1 to 1:1:90, preferably from about 10:1:1 to 1:10:1 to 1:1:10 and more preferably from 3:1:1 to 1:3:1 to 1:1:3.

It is yet a further object to provide a method and pharmaceutical formulation (medicament) for the effective treatment of pain in patients afflicted with primary and secondary dysmenorrhea by augmenting the analgesic effect of tramadol or its analog.

The invention is directed to the surprising and unexpected synergy obtained via the administration of a NMDA receptor antagonist together with an anticonvulsant and/or a tricyclic anti-depressant and tramadol or its analog.

The present invention is related in part to analgesic pharmaceutical compositions comprising a NMDA receptor antagonist together with an anticonvulsant and/or a tricyclic anti-depressant and tramadol or its analog. The pharmaceutical compositions can be administered intravenously, intrathecally, orally, via controlled release implant or pump, parenterally, sublingually, rectally, topically, via inhalation, etc.

The invention allows for the use of lower doses of a tramadol or its analog or a NMDA receptor antagonist, (referred to as apparent “one-way synergy” herein), or lower doses of both drugs (referred to as “two-way synergy” herein) than would normally be required when either drug is used alone. By using lower amounts of either or both drugs, the side effects associated with effective pain management in humans and other species are significantly reduced.

In certain preferred embodiments, the invention is directed in part to synergistic combinations of dextromethorphan or other NMDA receptor antagonist in an amount sufficient to render a therapeutic effect together with an anticonvulsant and/or a tricyclic anti-depressant and tramadol or its analog, such that an analgesic effect is attained which is at least about 5 (and preferably at least about 10) times greater than that obtained with the dose of tramadol or its analog alone. This is exemplified by the apparent fact that patients with diabetic neuropathy and fibromyalgia, who could not get even 30-40% reduction in pain even with the administration of 400 mg of tramadol per day, can have shown 90-100% pain relief with the inventive composition containing 35 mg of tramadol, 35-45 mg of dextromethorphan and 90 mg of gabapentin over a period of 12-16 hours.

In certain embodiments, the synergistic combination provides an analgesic effect which is up to about 10 to 20 times greater than that obtained with the dose of an anticonvulsant and/or a tricyclic anti-depressant if administered as a single agent. In such embodiments, the synergistic combinations display what is referred to herein as an “apparent mutual synergy”, meaning that the dose of NMDA antagonist and anticonvulsant and/or a tricyclic anti-depressant synergistically potentiates the effect of tramadol or its analog and the dose of tramadol or its analog appears to potentiate the effect of the NMDA antagonist and the anticonvulsant and/or a tricyclic anti-depressant.

The combination of NMDA antagonist, anticonvulsant and/or a tricyclic anti-depressant and tramadol or its analog can be administered in a single dosage form. Alternatively the combination can be administered separately, preferably concomitantly.

In certain preferred embodiments, the synergism exhibited between the three types of drugs, is such that the dosage of tramadol or its analog would be sub-therapeutic if administered without the dosage of the NMDA antagonist and anticonvulsant and/or a tricyclic anti-depressant. This synergy can be further augmented by the addition of a fourth drug. Similarly, in certain preferred embodiments wherein the pharmaceutical composition comprises a combination of NMDA antagonist, anticonvulsant and/or a tricyclic anti-depressant and tramadol or its analog and is essentially free of a NSAID or acetaminophen, the dosage of tramadol or its analog would be sub-therapeutic if administered without the dosage of the NMDA antagonist and anticonvulsant and/or a tricyclic anti-depressant. In other preferred embodiments, the present invention relates to a pharmaceutical composition comprising an analgesically effective dose of tramadol or its analog together with a dose of a NMDA antagonist and an anticonvulsant and/or a tricyclic anti-depressant effective to augment the analgesic effect of tramadol or its analog, or a composition essentially free of a NSAID or acetaminophen and comprising an analgesically effective dose of tramadol or its analog together with a dose of a NMDA antagonist effective to augment the analgesic effect of tramadol or its analog

It is believed that in actuality these combinations exhibit two-way synergism, meaning that the NMDA antagonist and the anticonvulsant and/or a tricyclic anti-depressant potentiate the effect of tramadol or its analog, and tramadol or its analog, potentiates the effect of the NMDA antagonist and the anticonvulsant and/or a tricyclic anti-depressant. Thus, other embodiments of the invention relate to combinations of NMDA antagonist, anticonvulsant and/or a tricyclic anti-depressant and tramadol or its analog where the dose of each drug is reduced due to the synergism demonstrated between the drugs, and the analgesia derived from the combination of drugs in reduced doses is surprisingly and strongly enhanced. The two-way synergism is not always readily apparent in actual dosages due to the potency ratio of tramadol or its analog to the NMDA antagonist and anticonvulsant and/or a tricyclic anti-depressant. By this we mean that tramadol or its analog generally displays unexpectedly enhanced analgesic potency.

In certain preferred embodiments, the invention is directed to pharmaceutical formulations comprising a NMDA antagonist such as dextromethorphan and magnesium, an anticonvulsant and/or a tricyclic anti-depressant in an amount sufficient to render a therapeutic effect, and a therapeutically effective or sub-therapeutic amount of tramadol or its analog. Preferably, tramadol or its analog is selected from the group consisting of tramadol, its metabolites thereof, salts thereof, recemates thereof, and complexes thereof.

In certain preferred embodiments, the invention is directed to pharmaceutical formulations comprising a NMDA antagonist such as dextromethorphan and magnesium, and an anticonvulsant and/or a tricyclic anti-depressant in an amount sufficient to render a therapeutic effect together with a therapeutically effective or sub-therapeutic amount of tramadol or its analog. Preferably, tramadol or its analog is selected from the group consisting of tramadol, its metabolites thereof, salts thereof, recemates thereof, and complexes thereof.

In certain embodiments, the invention is directed to pharmaceutical formulations comprising a NMDA antagonist such as dextromethorphan and magnesium, and an anticonvulsant and/or a tricyclic anti-depressant in an amount sufficient to render a therapeutic effect together with a dose of tramadol or its analog that is analgesic if administered without the NMDA antagonist and the anticonvulsant and/or a tricyclic anti-depressant. Preferably, tramadol or its analog is tramadol. The dose of tramadol is preferably from about 30 to about 100 mg.

The invention further relates to a method of effectively treating pain in humans suffering from primary and secondary dysmenorrhea, comprising administration to a human or mammalian patient a therapeutically effective amount of a NMDA antagonist and an anticonvulsant and/or a tricyclic anti-depressant together with a dose of tramadol or its analog, such that the combination provides an analgesic effect which is at least about 5, and preferably at least about 10, times greater than that obtained with the dose of tramadol or its analog alone. In certain embodiments, the synergistic combination provides an analgesic effect which is up to about 30 to 40 times greater than that obtained with the dose of tramadol or its analog alone.

In certain preferred embodiments, the doses of the NMDA antagonist, anticonvulsant and/or a tricyclic anti-depressant and tramadol or its analog are administered orally. In further preferred embodiments the doses of the NMDA antagonist, anticonvulsant and/or a tricyclic anti-depressant and tramadol or its analog are administered in a single oral dosage form. In certain preferred embodiments, the dose of tramadol or its analog would be sub-therapeutic if administered without the dose of the NMDA antagonist and the anticonvulsant and/or a tricyclic anti-depressant. In other preferred embodiments, the dose of tramadol or its analog is effective to provide analgesia alone, but the dose of tramadol or its analog provides at least a five fold greater analgesic effect than typically obtained with that dose of tramadol or its analog alone.

The invention further relates to the use of a pharmaceutical combination of a NMDA antagonist(s) together with a tramadol or its analog and an anticonvulsant and/or a tricyclic anti-depressant to provide effective pain management in humans afflicted with primary and secondary dysmenorrhea. The instant invention is a method of using a pharmaceutical combination in the treatment of pain, especially for treatment of pain associated with primary and secondary dysmenorrhea.

The invention further relates to the use of a NMDA antagonist in the manufacture of a pharmaceutical preparation containing a NMDA antagonist, an anticonvulsant and/or a tricyclic anti-depressant and tramadol or its analog for the treatment of pain.

The invention further relates to the use of a tramadol or its analog in the manufacture of a pharmaceutical preparation containing a NMDA antagonist, an anticonvulsant and/or a tricyclic anti-depressant, and tramadol or its analog for the treatment of pain of chronic, intermittent or acute nature.

The invention is also directed to a method for providing effective pain management in humans suffering from primary and secondary dysmenorrhea, comprising administration of either an analgesically effective or sub-therapeutic amount of a tramadol or its analog, administration of an effective amount of an anticonvulsant and/or a tricyclic anti-depressant in an amount effective to augment synergistically the analgesic effect provided by said tramadol or its analog, and administration of an effective amount of a NMDA antagonist such as dextromethorphan in an amount effective to augment synergistically the analgesic effect provided by said tramadol or its analog. The NMDA antagonist and anticonvulsant and/or a tricyclic anti-depressant can be administered prior to, concurrently with, or after administration of tramadol or its analog, as long as the dosing interval of NMDA antagonist overlaps with the dosing interval of tramadol or its analog and/or its analgesic effects.

The anticonvulsant and/or a tricyclic anti-depressant can be administered prior to, concurrently with, or after administration of tramadol or its analog and a NMDA antagonist, as long as the dosing interval of the anticonvulsant and/or a tricyclic anti-depressant and a NMDA antagonist overlaps with the dosing interval of tramadol or its analog and/or its analgesic effects. In other words, according to the method of the present invention, in certain preferred embodiments the NMDA antagonist and the anticonvulsant and/or a tricyclic anti-depressant need not be administered in the same dosage form or even by the same route of administration as tramadol or its analog. Rather, the method is directed to the surprising synergistic and/or additive analgesic benefits obtained in humans or other mammals, when analgesically effective levels of tramadol or its analog have been administered to a human or other mammals, and, prior to or during the dosage interval for tramadol or its analog or while the human or other mammal is experiencing analgesia, an effective amount of NMDA antagonist and anticonvulsant and/or a tricyclic anti-depressant to augment the analgesic effect of tramadol or its analog is administered. If the NMDA antagonist and the anticonvulsant and/or a tricyclic anti-depressant are administered prior to the administration of tramadol or its analog, it is preferred that the dosage intervals for the two drugs overlap, i.e., such that the analgesic effect over at least a portion of the dosage interval of tramadol or its analog is at least partly coincident with the period of useful therapeutic effect of the NMDA antagonist and the anticonvulsant and/or a tricyclic anti-depressant.

In an additional method of the invention, the surprising synergistic and/or additive benefits obtained in humans suffering from primary and secondary dysmenorrhea are achieved when analgesically effective levels of a tramadol or its analog have been administered to a human during the time period of the therapeutic effect of a NMDA antagonist and an anticonvulsant and/or a tricyclic anti-depressant. Alternatively the method comprises the effective analgesia obtained when the human or other mammal is experiencing analgesia by virtue of the administration of a NMDA antagonist and an anticonvulsant and/or a tricyclic anti-depressant and an effective amount of a tramadol or its analog to synergistically augment the analgesic effect of tramadol or its analog.

In a further embodiment of the present invention, the invention comprises an oral solid dosage form comprising an analgesically effective amount of tramadol or its analog together with an amount of a NMDA antagonist and an anticonvulsant and/or a tricyclic anti-depressant which augment the effect of tramadol or its analog.

Optionally, the oral solid dosage form includes a sustained release carrier that effectuates the sustained release of tramadol or its analog, or both the tramadol or its analog and the NMDA antagonist when the dosage form contacts gastrointestinal fluid. The sustained release dosage form may comprise a multiplicity of substrates and carriers that include the drugs. The substrates may comprise matrix spheroids or may comprise inert pharmaceutically acceptable beads that are coated with the drugs. The coated beads are then preferably overcoated with a sustained release coating comprising the sustained release carrier. The matrix spheroid may include the sustained release carrier in the matrix itself, or the matrix may comprise a simple disintegrating or prompt release matrix containing the drugs, the matrix having a coating applied thereon which comprises the sustained release carrier. In yet other embodiments, the oral solid dosage form comprises a tablet core containing the drugs within a normal or prompt release matrix with the tablet core being coated with a sustained release coating comprising the sustained release carrier.

In yet further embodiments, the tablet or capsule contain the drugs within a sustained release matrix comprising the sustained release carrier. In yet further embodiments, the tablet contains tramadol or its analog within a sustained release matrix, and the NMDA antagonist and anticonvulsant and/or a tricyclic anti-depressant coated into the tablet as an immediate release layer.

In many preferred embodiments of the invention, the pharmaceutical compositions containing the NMDA antagonist, an anticonvulsant and/or a tricyclic anti-depressant and tramadol or its analog set forth herein are administered orally. Such oral dosage forms may contain one or all of the drugs in immediate or sustained release form. For ease of administration, it is preferred that the oral dosage form contains all the three drugs. The oral dosage forms may be in the form of tablets, troches, lozenges, aqueous, solid or semi-solid solutions or mixtures, or oily suspensions or solutions, dispersible powders or granules, emulsions, multiparticulate formulations, syrups, elixirs, and the like.

In other embodiments, a pharmaceutical composition containing the NMDA antagonist, anticonvulsant and/or a tricyclic anti-depressant and tramadol or its analog can be administered in dosage form as a topical preparation, a solid state and or depot type transdermal delivery device(s), a suppository, a buccal tablet, or an inhalation formulation such as a controlled release particle formulation or spray, mist or other topical vehicle, intended to be inhaled or instilled into the sinuses.

The pharmaceutical compositions containing the NMDA antagonist, anticonvulsant and/or a tricyclic anti-depressant and/or tramadol or its analog set forth herein may alternatively be in the form of microparticles such as microcapsules, microspheres and the like, which may be injected or implanted into a human patient, or other implantable dosage forms known to those skilled in the art of pharmaceutical formulation.

Similarly, pharmaceutical compositions essentially free of a NSAID or acetaminophen and comprising a combination of a NMDA antagonist, an anticonvulsant and/or a tricyclic anti-depressant and a tramadol or its analog can be prepared in solid oral dosage forms or other dosage forms as described above. Accordingly, the pharmaceutical compositions can be administered orally, by means of an implant, parenterally, sub-dermally, sublingually, rectally, topically, or via inhalation.

Another embodiment of the invention is directed to a method of alleviating pain associated with primary and secondary dysmenorrhea without the use of a narcotic analgesic. The method comprises administering to a patient a pharmaceutical composition comprising a NMDA antagonist, an anticonvulsant and/or a tricyclic anti-depressant and tramadol or its analog, or comprising a pharmaceutical composition essentially free of a NSAID or acetaminophen and comprising a combination of a NMDA antagonist, an anticonvulsant and/or a tricyclic anti-depressant and tramadol or its analog. In accordance with this embodiment, the active agents can be administered either together or separately, and the patient is not administered a narcotic analgesic.

### BRIEF DESCRIPTION OF THE DRAWING

The following drawings form part of the present specification and are included to further demonstrate certain aspects of the present invention. The invention may be better understood by reference to one or more of these drawings in combination with the detailed description of specific embodiments presented herein.

FIG. 1 provides the chemical structure of Dextromethorphan.

FIG. 2 provides the chemical structure of Tramadol.

FIG. 3 provides the chemical structures of certain Gabapentin and Pregabalin.

FIG. 4 provides chemical structure of Venlafaxine.

FIG. 5 provides chemical structure Amitriptyline.

FIG. 6 provides chemical structure of Milnacipran.

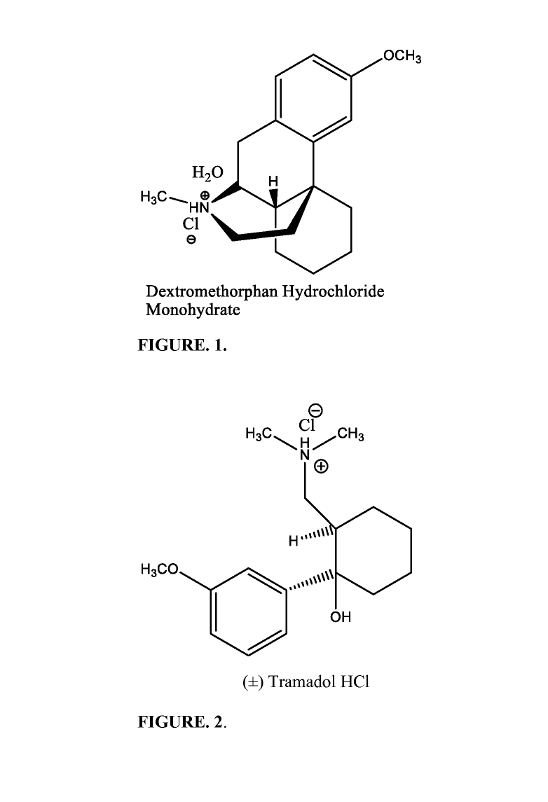

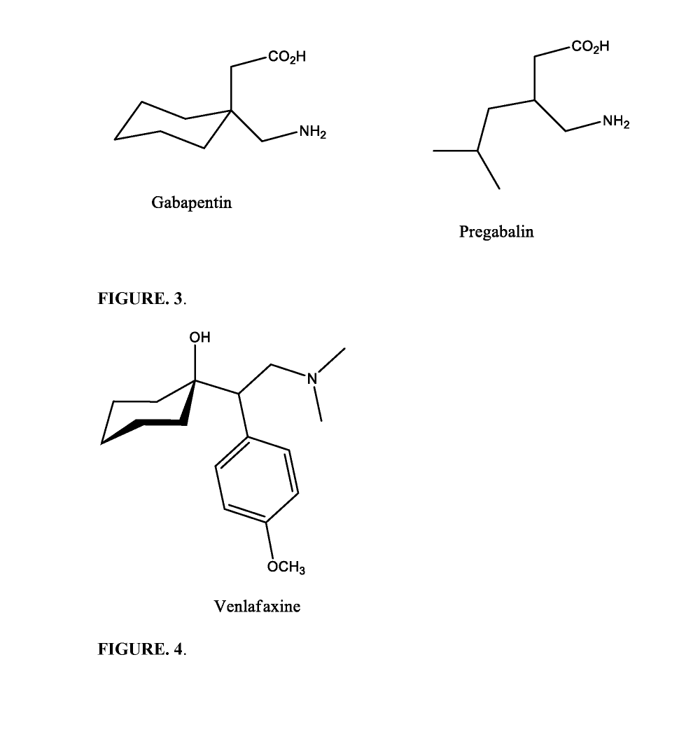

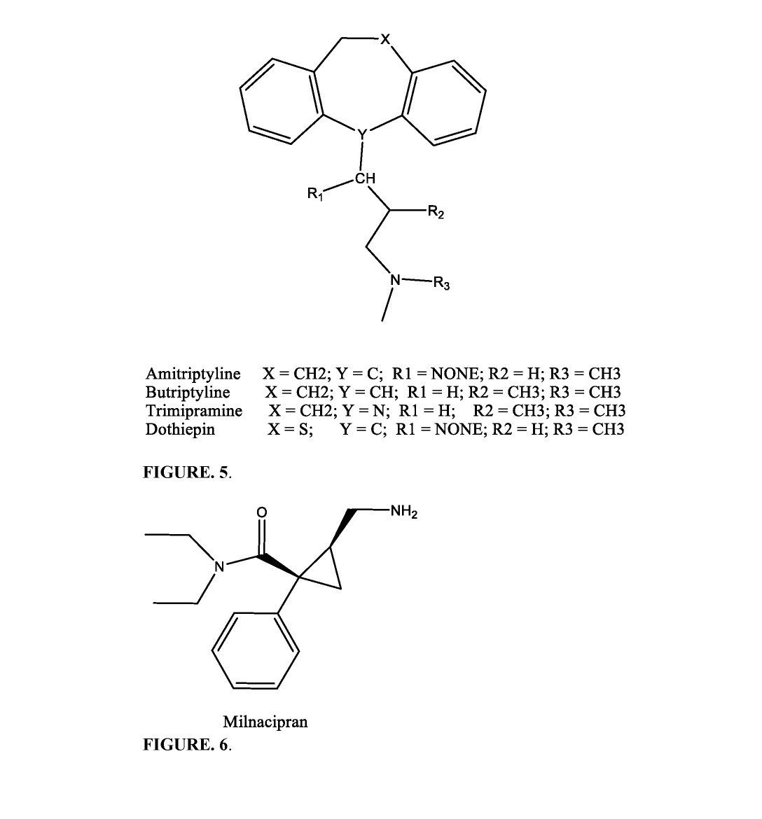

### DETAILED DESCRIPTION OF THE INVENTION

### Definition of Terms

It should be understood that for purposes of the present invention, the following terms have the following meanings:

The term “effective analgesia” is defined for purposes of the present invention as a satisfactory reduction in or elimination of pain, along with the production of a tolerable level of side effects, as determined by the human patient.

The term “pain management or effective pain management” is defined for the purposes of the present invention as the objective evaluation or opinion of a human patient's response (pain experienced versus side effects) to analgesic treatment by a physician as well as subjective evaluation of therapeutic treatment by the patient undergoing such treatment. The skilled artisan will understand that effective analgesia will vary widely according to many factors, including individual patient variables.

The term “tramadol or its analog” is defined for purposes of the present invention as the drug in its base form, or a pharmaceutically acceptable salt or complex thereof. Even though it is known that the pure enantiomers of tramadol have a differing pharmaceutical profiles and effects when compared to the racemate as discussed in the background of the invention, it should be understood for the purpose of the invention, both the optical isomers and the recemic mixtures of tramadol will be referred simply as “tramadol or its analog”.

The term “NMDA antagonist” as used herein is intended to encompass compounds that deactivate the NMDA receptor. The NMDA receptor is a ligand gated ion channel that allows for the transfer of electrical signals between neurons in the brain and in the spinal column. For electrical signals to pass, the NMDA receptor must be open. To remain open, an NMDA receptor must bind to glutamate and to glycine. An NMDA receptor that is bound to glycine and glutamate and has an open ion channel is called “activated”. NMDA antagonists fall into four categories: Competitive antagonists, which bind to and block the binding site of the neurotransmitter glutamate; glycine antagonists, which bind to and block the glycine site; noncompetitive antagonists, which inhibit NMDARs by binding to allosteric sites; and uncompetitive antagonists, which block the ion channel by binding to a site within it. Examples of NMDA receptor antagonists include, but not limited to, dextromethorphan, magnesium, dextrorphan, ketamine, amantadine, memantine, eliprodil, ifenprodil, phencyclidine, MK-801, dizocilpine, CCPene, flupirtine, or derivatives or salts thereof. Even though, magnesium exerts various physiological effects, for the purpose of teaching the present invention, it will be simply referred as NMDA receptor antagonist.

The term “dextromethorphan” is defined for purposes of the present invention as the drug in its base form, or a pharmaceutically acceptable salt or complex thereof.

The term “magnesium” is defined for purposes of the present invention as the pharmaceutically acceptable salt of magnesium which include, but not limited to, magnesium chloride, magnesium sulfate, magnesium gluconate, magnesium citrate, magnesium aspartate, magnesium lactate, magnesium levulinate, magnesium pidolate, magnesium orotate, magnesium oxide and magnesium malate. The amount of magnesium refers to the amount of elemental magnesium present in a pharmaceutically acceptable salt of magnesium.

The term “anticonvulsant” as used herein is intended to encompass compounds which possess anti-epileptic activity and some of them bind to the family of proteins called α₂δ. Examples of such compound include, but not limited to, sodium channel blockers such as carbamazepine, phenytoin, oxcarbazepine, lamotrigine and zonisamide, benzodiazepine analogs, valproate, glutamate blockers such as felbamate and topiramate, levetiracetam, gabapentin, derivatives or analogs of gabapentin or any compounded mixture thereof (see FIG. 2). Examples of analog of gabapentin include, but not limited to, pregabalin, 3-methyl-gabapentin, [(1R,5R,6S)-6-(Aminomethyl)bicyclo[-3.2.0]hept-6-yl]acetic acid, 3-(1-Aminomethyl-cyclohexylmethyl)-4H-[1,2,4]-oxadiazol-5-one, C-[1-(1H-Tetrazol-5-ylmethyl)-cycloheptyl]-methylamine, (3S,4S)-(1-Aminomethyl-3,4-dimethyl-cyclopentyl)-acetic acid, (1a,3a,5a)(3-amino-methyl-bicyclo[3.2.0]hept-3-yl)-acetic acid, (3S,5R)-3-Aminomethyl-5-methyl-octanoic acid, (3S,5R)-3-amino-5-methyl-heptanoic acid, (3S,5R)-3-amino-5-methyl-nonanoic acid and (3S,5R)-3-Amino-5-methyl-octanoic acid, or a pharmaceutically acceptable salt thereof.

The term “tricyclic anti-depressant” (abbreviation TCA) as used herein is intended to encompass a class of anti-depressant drugs and these drugs are named after their molecular structures, which contain three rings of atoms (See FIG. 4). Prominent among the tricyclic anti-depressants are the linear tricyclics, e.g., imipramine, desipramine, amitriptyline, nortriptyline, protriptyline, doxepin, ketipramine, mianserin, dothiepin, amoxapine, dibenzepin, melitracen, maprotiline, flupentixol, azaphen, tianeptine and related compounds showing similar activity. Angular tricyclics include indriline, clodazone, nomifensin, and related compounds. A variety of other structurally diverse anti-depressants, e.g., iprindole, wellbatrin, nialamide, milnacipran, phenelzine and tranylcypromine have been shown to produce similar activities. They are functionally equivalent to the tricyclic anti-depressants and are therefore included within the scope of the invention. Thus, the term tricyclic anti-depressant is intended by the present inventors to embrace the broad class of anti-depressants described above together with related compounds sharing the common property that they all possess anti-depressant activity.

The term “an anticonvulsant and/or a tricyclic anti-depressant” as used herein is intended to encompass either a combination of an anticonvulsant and a tricyclic anti-depressant or an anticonvulsant alone or a tricyclic anti-depressant alone.

The term “pain relieving” is generally defined herein to include the expressions “pain-suppressing”, “pain-reducing”, and “pain-inhibiting” as the invention is applicable to the alleviation of existing pain, as well as the suppression or inhibition of pain which would otherwise ensue from the imminent pain-causing event.

The term “sustained or controlled release” is defined for purposes of the present invention as the release of the drug (tramadol or its analog) from the transdermal formulation at such a rate that blood (plasma) concentrations (levels) of the drugs are maintained within the therapeutic range that is above the minimum effective analgesic concentration or “MEAC”, but below toxic levels over a period of time of several hours to several days.

The term “steady state” means that the blood plasma time/concentration curve for a given drug level has been substantially stable within a set range from dose to dose.

The term “minimum effective analgesic concentration” or “MEAC” is defined for purposes of this invention as the minimum effective therapeutic blood plasma level of the drug at which at least some pain relief is achieved in a given patient. It will be well understood by those skilled in the medical art that pain measurement is highly subjective and great individual variations may occur among patients.

It must be noted that, as used in this specification, the singular forms “a,” “an” and “the” include plural referents unless the context clearly dictates otherwise. Thus, for example, reference to “a pharmacologically active agent” includes a combination of two or more pharmacologically active agents, and the like.

As used herein in the claim(s), when used in conjunction with the word “comprising”, the words “a” or “an” may mean one or more than one. As used herein “another” may mean at least a second or more.

The term “NSAID” refers to non-steroidal anti-inflammatory drug. NSAIDs include the salicylates such as salicylamide and acetylsalicylic acid (aspirin). Non-aspirin NSAIDs include para-aminophenol derivatives such as phenacetin, the pyrazole derivatives such as antipyrine, aminopyrine, dypyrone, nefenamic acid, indomethacin, methimazole, paracetamol, diclophenac sodium/potassium, ibuprofen, naproxen, and ketorolac tromethamine. The analgesic acetaminophen is often categorized as a NSAID even though the compound does not exhibit significant anti-inflammatory activity. Unless otherwise indicated, acetaminophen will be referred to herein as a NSAID.

The term “chronic pain” means pain associated with an idiopathic or undiagnosed or an undiagnosible disease, disorder or condition, or pain associated with any one of: myofascial pain syndrome, trigger points, tender points, thorasic outlet syndrome, complex regional pain syndrome, reflex sympathetic dystrophy (RSD), sympathetically maintained pain (SMP), diabetic neuropathy syndrome (DNS); chronic pain associated with fibromyalgia syndrome (FMS), multiple sclerosis (MS); chronic pain associated with traumatic injury to the peripheral nervous system; chronic pain resulting from herpes zoster (also known as shingles, or post-herpetic neuropathy) or similar infections that attack and damage nerve fibers or endings; post-operative pain, which arises after surgery and then lingers far beyond a normal convalescent period; pain associated with nerve and root damage, such as pain associated with peripheral nerve disorders, including, nerve entrapment and brachial plexus avulsions, amputation, peripheral neuropathies, tic douloureux, atypical facial pain, nerve root damage, and arachnoiditis syndrome, in which an amputee suffers from feelings of pain or discomfort that seems to originate in the missing limb (“phantom limb” pain); pain associated with carcinoma, often referred to as cancer pain; neuropathic pain associated with chemotherapy treatment; central nervous system pain, including pain due to spinal cord or brain stem damage; low back pain; sciatica; headache, including migraine, chronic tension headache, cluster headache, temporomandibular disorder (TMJ) pain and maxillary sinus pain; complex regional pain syndromes, including reflex sympathetic dystrophy and causalgia, or from burn injury; the chronic pain associated with hyperesthesia, allodynia, hyperalgesia, deafferentation pain, sympathetically maintained pain, non-nociceptive chronic pain. It should be noted that eventhough the present invention relates to pain assoictaed with primary and secondary dysmenorrhea which may not be chronic in nature, the chronic pain is included for the purpose of describing the present invention.

The term “acute pain” refers to short-term pain. Acute pain may occur as a result of trauma, surgery, medical procedures, child birth, primary and secondary dysmenorrhea and/or brief disease states. Acute pain can be experienced as a physical sensation and may be felt as stabbing, burning, twisting, tearing, or squeezing.

The term “about” as used herein means±10% of the indicated numerical value.

### Description of the Applications of the Invention

The pharmacological management of acute pain and chronic pain syndromes has been traditionally based on various regimens of opiates and their congeners or NSAIDs. All opiates have side effects, of which the most dangerous are respiratory and cardiovascular depression associated with excessive sedation. NSAIDs may also induce side effects such as exacerbation of bleeding tendencies and the impairment renal function. The search for alternative pain control strategies has focused on the N-methyl-D-aspartate (NMDA) receptors and their antagonists which were shown to alleviate somatic and neuropathic pain sensation in both animal and human models (Plesan et al, 1998 and Klepstad et al, 1990). The clinical utility of these agents stems from the high affinity binding of the drugs to NMDA receptors resulting in blockade of the NMDA receptors located at the junction where pain is generated by peripheral nociceptive stimuli and is thence conveyed to central receptors via A * and C sensory fibres (Woolf et al, 1993). From a clinical standpoint, the amounts of conventional pain killers that are needed for effective pain control would be much smaller. One of these compounds is dextromethorphan (DM), a low affinity, non-competitive NMDA receptor antagonist that has a long history of clinical safety as a cough suppressant (Bem et al, 1992).

To date DM, ketamine and amantadine are the only drugs with NMDA receptor antagonistic properties that are FDA approved drugs for clinical use. However, due to the high affinity of ketamine to its receptors and its related dysphoric effects, together with the need to administer it intravenously, research in pain control has turned its focus to DM as the preferred NMDA antagonist for clinical use.

Also, unlike opiates, DM has an established safety record, i.e., the therapeutic cough suppressant dose (1-2 mg·kg⁻¹·dy⁻¹) has no major opiate like respiratory or hemodynamic side effects, neither does it induce histamine release complications.

### Elaboration of the Properties of the Preferred Active Ingredients

Satisfactory pain control achieved with the least amount of opiates has always been an important goal in view of both the psychological and somatic dependence these drugs may induce and the often intolerable side effects that may follow their extensive use. Moreover, in view of the contention that persistent NMDA receptor activation can evoke central hyperexcitability that can lead to secondary pain, proper pain control should both modulate primary pain sensation and preempt an analgesic state that would prevent acute pain from progressing into chronic pain. This concept of preemptive analgesia (i.e., reducing pain sensation in advance) is feasible via NMDA modulation, as had been demonstrated by the administration of opiates and ketamine to patients before surgery (Tverskoy M, et al. Preemptive effect of fentanyl and ketamine on postoperative pain and wound hyperalgesia. Anesth Analg 1994; 78: 205-9). Importantly, this neuropharmacological receptor conditioning is also beneficial for reducing the need for additional doses of opiates post-operatively. In addition, while the neurovegetative stimulation and adrenergic overproduction that accompany the continuous neurally transmitted acute and, to a greater extent, secondary pain are clearly detrimental to all patients, they may be particularly harmful for cardiac patients. In this regard, the preemptive approach is an especially promising and beneficial one. The use of DM may, therefore, become an established component in protocols of treating pain and of alleviating the accompanying neurovegetative phenomena. Finally, the bioavailability of DM administered orally makes it much more convenient than the other anti-NMDA drugs, all of which are administered by injection, such as ketamine. As a potential morphine sparing agent for pain, the use of DM was shown to be efficient and well tolerated (Henderson et al, 1999).

It is noteworthy that NMDA receptor antagonists, including DM, are not in themselves anti-nociceptive (Ilkjaer S, et al. Effect of systemic N-methyl-D-aspartate receptor antagonist (dextromethorphan) on primary and secondary hyperalgesia in humans. Br J Anaesth 1997; 79:600-5) but rather they inhibit central sensitization and, thus, the perception of primary and secondary pain. The preemptive use of these antagonists, while blunting the development of a central sensitization of a nociceptive stimulus (Yamamoto T, Yaksh T L. Comparison of the antinociceptive effects of pre-and posttreatment with intrathecal morphine and MK-801, a NMDA antagonist, on formalin test in rat. Anesthesiology 1992; 77: 757-63), still requires the use of an analgesic for complete abolition of pain perception.

Additional substances that block a major intracellular consequence of NMDA receptor activation and as such are useful in the practice of the invention include inhibitors of calmodulin such as the phenothiazines, in particular, chlorpromazine, chlorpromazine sulfoxide, prochlorperazine dimaleate, perphenazine, trifluoperazine, fluphenazine, fluphenazine enanthate, fluphenazine decanoate, thioridazine, mesoridazine besylate, piperacetazine, acetophenazine dimaleate, carphenazine dimaleate, butaperazine dimaleate and phenothiazine sulfoxide; naphthalenesulfonamides such as N-(6-aminohexyl)-5-chloro-1-naphthalene sulfonamide, N-(6-aminohexyl)-5-chloro-2-naphthalene sulfonamide and N-(6-aminohexyl)-5-bromo-2-naphthalene sulfonamide; 4-substituted-4H,6H-pyrrolo[1,2-a][4,1]benzoxazepines such as 1,3-dihydro-1-{1-[(4-methyl-4H,6H-pyrrolo[1,2-a][4,1]benzoxazepin-4-yl)methyl]-4-piperidinyl}-2H-benzimidazol-2-one; benzhydryls such as N-[2](diphenylmethylthioethyl]-2-(trifluoromethyl)-benzeneethanamine, N-[2-(bis(4-fluorophenyl)methylthio)-)ethyl]-2-(trifluoromethyl)benzene ethanamine and N-(bis(4-fluorophenyl)methylthio)ethyl]-3-(trifluoromethyl)benzene ethanamine; tricyclic antidepressant drugs such as imipramine, 2-chloroimipramine and amitriptyline; penfluridol; haloperidol; pimozide; clozapine; calmidazolin; and, mixtures and pharmaceutically acceptable salts of any of the foregoing.

Dextromethorphan and its active metabolite dextrorphan bind to the N-Methyl-D-Aspartate (NMDA) glutamate and nicotine/neuronal nicotinic receptors as inhibitors. Dextromethorphan and dextrorphan also bind to the receptor-gated (NMDA receptor mediated) and voltage-gated calcium channels, and the voltage-gated sodium channels as a blocker. Through these bindings, dextromethorphan and dextrorphan modulates the glutamate pathway in the central nervous system (CNS) and modulate most of the excitatory synaptic transmission. Dextromethorphan and dextrorphan also bind to the sigma receptors which are found in high concentrations in limbic and motor areas of the CNS sensory processing such as the dorsal root ganglia and the nucleus tractus solitarus (NTS). In addition, Dextromethorphan inhibits the reuptake of 5-HT (serotonin) and norepinephrine, thus modulating the monamine pathways.

Tramadol and its active metabolite M1, modulate neuronal pathways via contributions from both opioid (predominantly at the μ-opioid receptor) and non-opioid probably related to its inhibition of neuronal release or reuptake of norepinephrine and serotonin) mechanisms at therapeutic doses. Both mechanisms contribute to the effect of tramadol in vivo, leading to the suggestion that tramadol is a novel centrally acting analgesic that mimics, in a single drug substance, the clinical practice of combining opioid analgesics with monoamine reuptake inhibitors. Opioid receptors presynaptically inhibit transmission of excitatory pathways. These pathways include acetylcholine, the catecholamines, serotonin, and substance P. The present working hypothesis is that the overall neuronal action of tramadol is dependent on the different pharmacologies of its enantiomers and, to some extent its metabolite, M1. The enantiomers appear to interact in a complementary and synergistic manner to produce antinociception, but only in an additive or counteractive manner on adverse-effect end-points. Hence, the favorable clinical profile of tramadol appears to be a consequence of the fortuitous interaction of the enantiomers and the metabolite M1 on the therapeutic endpoint, but not on adverse-effect endpoints.

Venlafaxine is a novel SSRI chemically unrelated to other SSRIs but chemically similar to the tramadol. The mechanism of the antidepressant action of venlafaxine in humans is believed to be the same as with other SSRIs, associated with its potentiation of neurotransmitter activity in the CNS as with other SSRIs: preclinical studies have shown that venlafaxine and its active metabolite, O-desmethylvenlafaxine (ODV), are potent inhibitors of neuronal serotonin and norepinephrine reuptake and weak inhibitors of dopamine reuptake. That venlafaxine is analgesia is seen in studies in animals that show that venlafaxine is effective in reversing chronic neuropathic pain secondary to thermal hyperalgesia, and additionally is effective in treating the hyperalgesia of neuropathic pain due to chronic sciatic nerve constriction injury in rats. The antinociceptive effect of venlafaxine is mainly influenced by the κ- and δ-opioid receptor subtypes combined with the α2-adrenergic receptor. These results suggest a potential use of venlafaxine in the management of some pain syndromes.

Gabapentin (GBP; Neurontin®) is an anticonvulsant that has found increased utility for the treatment of clinical neuropathic pain. Gabapentin interacts with both the α_{2δ-}1 and α_{2δ-}2 subunits which are voltage-gated calcium channel thus blocking calcium influx into the neuronal cells. A specific role for α₂δ in neuropathic pain is due to the fact that an increase in α₂δ expression in the dorsal root ganglion ipsilateral to the peripheral nerve injury that corresponded to the development of tactile allodynia. In addition, gabapentin increases brain extracellular GABA levels in both rat and human studies which is partially responsible for its effectiveness for neuropathic pain, since the pathology associated with this condition includes disruption of tonic inhibitory GABAergic transmission.

Amitriptyline is a tricyclic agent used for the treatment of major depression. Amitriptyline, nortriptyline, and desipramine have been established as analgesics independent of their antidepressant effects. Although their mechanism of analgesic action has not been clearly defined, tricyclic antidepressants are thought to have an inhibitory effect on nociceptive pathways by blocking the reuptake of serotonin and norepinephrine (Calissi 1995). Originally, the major mechanism of the analgesic effect of tricyclic antidepressants was believed to be related to serotonin reuptake inhibition. Animal models of peripheral neuropathic pain have shown that tricyclic antidepressants act as sodium channel blockers, similar to local anesthetic and antiarrhythmic agents.

Magnesium-depletion is specifically deleterious to neurons by causing NMDA-coupled calcium channels to be biased towards opening, because magnesium is nature's calcium channel blocker. The targets for glutamate binding to NMDA receptors are calcium and magnesium ion channels and to a lesser extent calcium and zinc channels. At normal neuronal resting membrane potentials, pores of the glutamate-gated ion channel are blocked by Mg2+ ions. The ion channel of the NMDA-receptor complex is subject to voltage-dependent regulation by magnesium ions. Normally operating NMDA receptors admit into neurons only the amount of Ca2+ that is vital to their function, but abnormally functioning NMDA receptors increase influx of cellular Ca2+ beyond manageable levels leading to the generation of toxic reactive oxygen species and of toxic amounts of nitric oxide (NO) radicals. It has been shown that NMDA receptor channel characteristics in the dorsal horn are altered by inflammation, and that the changes observed could contribute to the hyperalgesia and allodynia associated with tissue injury. Imbalances in Na+ and Cl⁻ gradients as well as Ca2+ overloading are also implicated in neuronal swelling and cell death, while depolarization of membranes relieves the Mg2+ block and allows Na+ and Ca2+ to enter. Certain drugs can act in place of magnesium including memantine and ketamine with each producing benefits in depression.

### Description of Alternative Ingredients

A non-limiting list of tramadol or its analog drugs which may be utilized in the present invention include any one of (1R,2R or 1S,2S)-(dimethylaminomethyl)-1-(3-methoxyphenyl)-cyclohexanol (tramadol), its N-oxide derivative (“tramadol N-oxide”), its O-desmethyl derivative (“O-desmethyl tramadol”), venlafaxine, (R/S)-1-[2-(dimethylamino)-1-(4-methoxyphenyl)ethyl]cyclohexanol and O-desmethylvenlafaxine or mixtures, stereoisomers, recemates, metabolites, salts or complexes thereof.

A non-limiting list of NMDA antagonist drugs which may be utilized in the present invention include dextromethorphan, magnesium, dextrorphan, ketamine, amantadine, memantine, eliprodil, ifenprodil, phencyclidine, MK-801, dizocilpine, CCPene, flupirtine, or derivatives, salts, metabolites or complexes thereof.

A non-limiting list of analogs of gabapentin which may be used in the present invention include gabapentin, pregabalin, 3-methyl gabapentin, [(1R,5R,6S)-6-(Aminomethyl)bicyclo[-3.2.0]hept-6-yl]acetic acid, 3-(1-Aminomethyl-cyclohexylmethyl)-4H-[1,2,4]-oxadiazol-5-one, C-[1-(1H-Tetrazol-5-ylmethyl)-cycloheptyl]-methylamine, (3S,4S)-(1-Aminomethyl-3,4-dimethyl-cyclopentyl)-acetic acid, (1a,3a,5a)(3-amino-methyl-bicyclo[3.2.0]hept-3-yl)-acetic acid, (3S,5R)-3-Aminomethyl-5-methyl-octanoic acid, (3S,5R)-3-amino-5-methyl-heptanoic acid, (3S,5R)-3-amino-5-methyl-nonanoic acid and (3S,5R)-3-Amino-5-methyl-octanoic acid, (1-aminomethyl-3-methylcyclohexyl)acetic acid, (1-aminomethyl-3-methylcyclopentyl)acetic acid, (S)-3-(aminomethyl)-5-methylhexanoic acid, 3-aminomethyl-5-methyl-hexanoic acid, (1-aminomethyl-3,4-dimethylcyclopentyl)acetic acid or a pharmaceutically acceptable salt thereof, or an ester or amide derivative thereof.

A non-limiting list of pharmaceutically acceptable salt of magnesium which may be used in the present invention include magnesium chloride, magnesium sulfate, magnesium gluconate, magnesium citrate, magnesium aspartate, magnesium lactate, magnesium levulinate, magnesium pidolate, magnesium orotate, magnesium oxide and magnesium malate.

A non-limiting list of a tricyclic anti-depressant which may be used in the present invention includes amitriptyline, butriptyline, amoxapine, clomipramine, desipramine, dothiepin, imipramine, dibenzepin, iprindole, lofepramine, nortriptyline, opipramol, protriptyline, tianeptine, milnacipran and trimipramine.

### Description of Quantitative Pharmacological Parameters of the Mixture

Preferred embodiments of the present invention are pain relieving preparations for oral administration that provide a combination of a NMDA antagonist or a pharmaceutically acceptable salt thereof, an anticonvulsant and/or a tricyclic anti-depressant or a pharmaceutically acceptable salt thereof, and a tramadol or its analog or a pharmaceutically acceptable salt thereof. The combination preferably provides a synergistic or at least additive effect for analgesic dosages.

Dosage levels of the NMDA antagonist on the order of from about 0.3 mg to about 3 mg per kilogram of body weight per day and anticonvulsant and/or a tricyclic anti-depressant on the order of from about 0.05 mg to about 3 mg per kilogram of body weight are therapeutically effective in combination with tramadol or its analog. Alternatively, about 10 mg to about 200 mg per patient per day of a NMDA antagonist and about 5 mg to about 300 mg per patient per day of anticonvulsant and/or a tricyclic anti-depressant are administered in combination with tramadol or its analog. For example, chronic pain may be effectively treated by the administration of from about 0.3 to 3 mg of the NMDA antagonist per kilogram of body weight per day, or alternatively about 30 mg to about 300 mg per patient per day.

The amount of NMDA antagonist that may be combined with the carrier materials to produce a single dosage form having NMDA antagonist, anticonvulsant and/or a tricyclic anti-depressant and tramadol or its analog in combination will vary depending upon the patient and the particular mode of administration. For example, a formulation intended for the oral administration of humans may contain from 10 mg to 300 mg of NMDA antagonist compounded with an appropriate and convenient amount of carrier material that may vary from about 5 to about 95 percent of the total composition. Unit dosages will generally contain between from about 10 mg to about 100 mg of a NMDA antagonist.

Tramadol or its analog can be provided in a sustained release oral dosage form with as the therapeutically active analgesic in an amount from about 25 mg to about 400 mg tramadol hydrochloride. Alternatively, the dosage form may contain molar equivalent amounts of other tramadol salts or of the tramadol base. The dosage form may contain a mixture of tramadol and a derivative of tramadol to provide a substantially equivalent therapeutic effect.

Preferred combinations of the invention comprise an effective amount of a NMDA antagonist selected from the group consisting of dextromethorphan and magnesium, an effective amount tramadol and an effective amount of anticonvulsant and/or a tricyclic anti-depressant.

The amount of anticonvulsant in the composition will be an amount sufficient to further enhance analgesia or to hasten its onset. In humans, this amount will typically be from about 10 to about 3600 mg (preferably 20 to 1000 mg), an amount generally sufficient to both hasten onset and enhance analgesia. The daily dosage of anticonvulsant again will generally not exceed 3600 mg. Of course, greater amounts can be used if tolerated by the patient.

The amount of tricyclic anti-depressant in the composition will be an amount sufficient to further enhance analgesia or to hasten its onset. In humans, this amount will typically be from about 1 to about 1000 mg (preferably 5 to 300 mg), an amount generally sufficient to both hasten onset and enhance analgesia. The daily dosage of tricyclic anti-depressant again will generally not exceed 300 mg. Of course, greater amounts can be used if tolerated by the patient.

In certain preferred embodiments according to the present invention, an oral dosage form is preferred which includes the following tramadol or its analog/NMDA antagonist/anticonvulsant combinations: Tramadol 35 mg plus 35 mg dextromethorphan plus 90 mg gabapentin; tramadol 35 mg plus 35 mg dextromethorphan plus 180 mg gabapentin; tramadol 35 mg plus 45 mg dextromethorphan plus 45 mg gabapentin or 50 mg of tramadol plus 30 mg of dextromethorphan plus 90 mg gabapentin; Tramadol 35 mg plus 45 mg dextromethorphan plus 90 mg gabapentin.

In another preferred embodiments according to the present invention, an oral dosage form is preferred which includes the following tramadol or its analog/NMDA antagonist/anticonvulsant combinations: Tramadol 35 mg plus 35 mg dextromethorphan plus 20 mg pregabalin; tramadol 35 mg plus 35 mg dextromethorphan plus 30 mg pregabalin; tramadol 35 mg plus 45 mg dextromethorphan plus 15 mg pregabalin or 50 mg of tramadol plus 30 mg of dextromethorphan plus 15 mg gabapentin; Tramadol 35 mg plus 45 mg dextromethorphan plus 30 mg pregabalin.

In another preferred embodiments according to the present invention, an oral dosage form is preferred which includes the following tramadol or its analog/NMDA antagonist/anticonvulsant/magnesium combinations: Tramadol 35 mg plus 35 mg dextromethorphan plus 90 mg gabapentin plus 24 mg of magnesium; tramadol 35 mg plus 30 mg dextromethorphan plus 100 mg gabapentin plus 24 mg of magnesium; tramadol 35 mg plus 45 mg dextromethorphan plus 45 mg gabapentin plus 24 mg of magnesium; 50 mg of tramadol plus 30 mg of dextromethorphan plus 90 mg gabapentin plus 24 mg of magnesium.

In certain preferred embodiments according to the present invention, an oral dosage form is preferred which includes the following tramadol or its analog/NMDA antagonist/tricyclic antidepressant combinations: Tramadol 35 mg plus 35 mg dextromethorphan plus 10 mg amitriptyline or milnacipran; tramadol 35 mg plus 45 mg dextromethorphan plus 5 mg amitriptyline or milnacipran; or 50 mg of tramadol plus 30 mg of dextromethorphan plus 10 mg amitriptyline or milnacipran.

In certain preferred embodiments according to the present invention, an oral dosage form is preferred which includes the following tramadol or its analog/NMDA antagonist/anticonvulsant and tricyclic antidepressant combinations: Tramadol 35 mg plus 35 mg dextromethorphan plus 90 mg gabapentin plus 10 mg amitriptyline or milnacipran; tramadol 35 mg plus 45 mg dextromethorphan plus 45 mg gabapentin plus 5 mg amitriptyline or milnacipran; tramadol 35 mg plus 45 mg dextromethorphan plus 45 mg gabapentin plus 10 mg amitriptyline or milnacipran; or 35 mg of tramadol plus 30 mg of dextromethorphan plus 90 mg gabapentin plus 10 mg amitriptyline or milnacipran.

In another preferred embodiments according to the present invention, an oral dosage form is preferred which includes the following tramadol or its analog/NMDA antagonist/anticonvulsant combinations: Tramadol 44 mg plus 24.0 mg magnesium plus 100 mg gabapentin; and tramadol 44 mg plus 40.8 mg magnesium plus 100 mg gabapentin.

The dosage administered will of course vary depending upon known factors such as the pharmacodynamic characteristics of each agent of the combination and its mode and route of administration and upon the age, health and weight of the patient. The dosage will also depend upon the nature and extent of symptoms, concurrent treatment, if any, frequency of treatment and the desired result. A composition comprising any of the above identified combinations of tramadol or its analog, gabapentin or analog of gabapentin and NMDA antagonist may be administered in divided doses ranging from 2 to 6 times per day or in a sustained release form that will provide a rate of release effective to attain the desired results.

The optimal NMDA antagonist to tramadol or its analog ratios can be determined by standard assays well known in the art for determining opiate and analgesic activity. For example, the phenyl-p-benzoquinone test may be used to establish analgesic effectiveness. The phenyl-p-benzoquinone induced writhing test in mice as described in Blumberg et al, 1965, Proc. Soc. Exp. Med. 118:763-766, hereby incorporated by reference, and known modifications thereof, is a standard procedure which may be used for detecting and comparing the analgesic activity of different classes of analgesic drugs with a good correlation with human analgesic activity. Data for the mouse, as presented in an isobologram, can be translated to other species where the orally effective analgesic dose of the individual compounds are known or can be estimated.

Without wishing to be bound to any particular theory, the Applicants believe that the compositions of this invention significantly reduce the pain associated with primary and secondary dysmenorrhea through modulating pain signals without affecting the uterus contractions as they are needed to shed the wste from the uterus.

### Elaboration of Preferred and Alternative Formulations and Vehicles

The present invention encompasses immediate release dosage forms of an effective analgesic amount of dextromethorphan, gabapentin or an analog of gabapentin and tramadol or its analog combination. An immediate release dosage form may be formulated as a tablet or multi-particulate that may be encapsulated. Other immediate release dosage forms known in the art can be employed.

Compositions of the invention present the opportunity for obtaining relief from moderate to severe pain. Due to the synergistic and/or additive effects provided by the inventive combination of tramadol or its analog, anticonvulsant and/or a tricyclic anti-depressant and NMDA antagonist, it may be possible to use reduced dosages of each of NMDA antagonist and tramadol or its analog. By using lesser amounts of other or both drugs, the side effects associated with each may be reduced in number and degree. Moreover, the inventive combination avoids side effects to which some patients are particularly sensitive.

The present invention encompasses a method of inhibiting NMDA receptor and treating primary and secondary dysmenorrhea comprising administering to a patient in need of such treatment a non-toxic therapeutically effective amount of the NMDA antagonist, anticonvulsant and/or a tricyclic anti-depressant and tramadol or its analog combination of the present invention.

Further, the combination of NMDA antagonist, anticonvulsant and/or a tricyclic anti-depressant and tramadol or its analog is useful as an alternative to conventional non-steroidal anti-inflammatory drugs or combinations of NSAIDS with other drugs particularly where such non-steroidal anti-inflammatory drugs may be contraindicated such as in patients with peptic ulcers, gastritis, regional enteritis, ulcerative colitis, diverticulitis or with a recurrent history of gastrointestinal lesions, GI bleeding, coagulation disorders including anemia such as hypoprothrombinemia, haemophilia or other bleeding problems, kidney disease and in those prior to surgery or taking anticoagulants.

The sustained release dosage forms of the present invention generally achieve and maintain therapeutic levels substantially without significant increases in the intensity and/or degree of concurrent side effects, such as nausea, vomiting, seizures or drowsiness, which are often associated with high blood levels of tramadol or its analogs. There is also evidence to suggest that the use of the present dosage forms leads to a reduced risk of drug addiction.

The combination of NMDA antagonist, anticonvulsant and/or a tricyclic anti-depressant and tramadol or its analog may be formulated to provide for an increased duration of analgesic action allowing once daily dosing. These formulations, at comparable daily dosages of conventional immediate release drug, are associated with a lower incidence in severity of adverse drug reactions and can also be administered at a lower daily dose than conventional oral medication while maintaining pain control.

The combination of NMDA antagonist, anticonvulsant and/or a tricyclic anti-depressant and tramadol or its analog can be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For parenteral application, particularly suitable are oily or aqueous solutions, as well as suspensions, emulsions, or implants, including suppositories. For oral application, particularly suitable are tablets, troches, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients which are suitable for the manufacture of tablets. Such excipients include, for example an inert diluent such as lactose, granulating and disintegrating agents such as cornstarch, binding agents such as starch, and lubricating agents such as magnesium stearate. The tablets may be uncoated or they may be coated by known techniques for elegance or to delay release of the active ingredients. Formulations for oral use may also be presented as hard gelatin capsules wherein the active ingredient is mixed with an inert diluent.

Aqueous suspensions that contain the aforementioned combinations of drugs and that such a mixture has one or more excipients suitable as suspending agents, for example pharmaceutically acceptable synthetic gums such as hydroxypropylmethylcellulose or natural gums. Oily suspensions may be formulated by suspending the aforementioned combinations of drugs in a vegetable oil or mineral oil. The oily suspensions may contain a thickening agent such as bees' wax or cetyl alcohol. A syrup, elixir, or the like can be used wherein a sweetened vehicle is employed. Injectable suspensions may also be prepared, in which case appropriate liquid carriers, suspending agents and the like may be employed. It is also possible to freeze-dry the active compounds and use the obtained lyophilized compounds, for example, for the preparation of products for injection.

### Controlled Release Dosage Forms

The NMDA antagonist, anticonvulsant and/or a tricyclic anti-depressant and tramadol or its analog combination can be formulated as a controlled or sustained release oral formulation in any suitable tablet, coated tablet or multiparticulate formulation known to those skilled in the art. The sustained release dosage form may optionally include a sustained released carrier which is incorporated into a matrix along with tramadol or its analog, or which is applied as a sustained release coating.

The sustained release dosage form may include the tramadol or its analog in sustained release form and the NMDA antagonist and anticonvulsant and/or a tricyclic anti-depressant in sustained release form or in immediate release form. The NMDA antagonist and anticonvulsant and/or a tricyclic anti-depressant may be incorporated into the sustained release matrix along with tramadol or its analog, incorporated into the sustained release coating; incorporated as a separated sustained release layer or immediate release layer, or may be incorporated as a powder, granulation, etc., in a gelatin capsule with the substrates of the present invention. Alternatively, the sustained release dosage form may have the NMDA antagonist in sustained release form and the tramadol or its analog and anticonvulsant and/or a tricyclic anti-depressant in sustained release form or immediate release form.

An oral dosage form according to the invention may be provided as, for example, granules, spheroids, beads, and pellets or pills. These formulations are hereinafter collectively referred to as “multiparticulates” and/or particles. An amount of the multiparticulates that is effective to provide the desired dose of tramadol or its analog over time may be placed in a capsule or may be incorporated in any other suitable oral solid form.

In one preferred embodiment of the present invention, the sustained release dosage form comprises such particles containing or comprising the active ingredient, wherein the particles have diameter from about 0.1 mm to about 2.5 mm, preferably from about 0.5 mm to about 2 mm.

In certain embodiments, the particles comprise normal release matrixes containing the tramadol or its analog with or without the NMDA antagonist and anticonvulsant and/or a tricyclic anti-depressant. These particles are then coated with the sustained release carrier. In embodiments where the NMDA antagonist and anticonvulsant and/or a tricyclic anti-depressant are immediately released, the NMDA antagonist and anticonvulsant and/or a tricyclic anti-depressant may be included in separate normal release matrix particles, or may be co-administered in a different immediate release composition which is either enveloped within a gelatin capsule or is administered separately. In other embodiments, the particles comprise inert beads that are coated with tramadol or its analog with or without the NMDA antagonist and anticonvulsant and/or a tricyclic anti-depressant. Thereafter, a coating comprising the sustained release carrier is applied onto the beads as an overcoat.

The particles are preferably film coated with a material that permits release of the tramadol or its analog or its salt, and if desired, the NMDA antagonist and anticonvulsant and/or a tricyclic anti-depressant at a sustained rate in an aqueous medium. The film coat is chosen so as to achieve, in combination with the other stated properties, a desired in vivo release rate. The sustained release coating formulations of the present invention should be capable of producing a strong, continuous film that is smooth and elegant, capable of supporting pigments and other coating additives, non-toxic, inert, and tack free.

### Coatings

The dosage forms of the present invention may optionally be coated with one or more materials suitable for the regulation of release or for the protection of the formulation. In one embodiment, coatings are provided to permit either pH dependent or pH independent release, e.g., when exposed to gastrointestinal fluid. A pH dependent coating serves to release the tramadol or its analog in desired areas of the gastro-intestinal (GI) tract, e.g., the stomach or small intestine, such that an absorption profile is provided which is capable of providing at least about twelve hour and preferably up to twenty four hour analgesia to a patient. When a pH independent coating is desired, the coating is designed to achieve optimal release regardless of pH changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine.

Formulations according to the invention that utilize pH dependent coatings to obtain formulations may also impart a repeat-action or pulsatile release effect whereby unprotected drug is coated over the enteric coat and is released in the stomach, while the remainder, being protected by the enteric coating, is released further down the gastrointestinal tract. Coatings which are pH dependent may be used in accordance with the present invention include shellac, cellulose acetate phthalate (CAP), polyvinyl acetate phthalate (PVAP), hydroxypropylmethylcellulose phthalate, and methacrylic acid ester copolymers, zein, and the like.

The substrate (e.g., tablet core bead, matrix particle) containing the tramadol or its analog (with or without the NMDA antagonist and anticonvulsant and/or a tricyclic anti-depressant) is coated with a hydrophobic material selected from (i) an alkylcellulose; (ii) an acrylic polymer, or (iii) mixtures thereof. The coating may be applied in the form of an organic or aqueous solution or dispersion. The coating may be applied to obtain a weight gain from about 2 to about 25% of the substrate in order to obtain a desired sustained release profile. Such formulations are described in detail in U.S. Pat. Nos. 5,273,760 and 5,286,493, hereby incorporated by reference in their entirety.

Other examples of sustained release formulations and coatings that may be used in accordance with the present invention include U.S. Pat. Nos. 5,324,351, 5,356,467, and 5,472,712, hereby incorporated by reference in their entirety.

### Alkylcellulose Polymers

Cellulosic materials and polymers, including alkylcelluloses, provide hydrophobic materials well suited for coating the beads according to the invention. Simply by way of example, one preferred alkylcellulosic polymer is ethylcellulose, although the artisan will appreciate that other cellulose and/or alkylcellulose polymers may be readily employed, singly or in any combination, as all or part of a hydrophobic coating according to the invention.

One commercially available aqueous dispersion of ethylcellulose is sold as Aquacoat™ (FMC Corp., Philadelphia, Pa., U.S.A.). Aquacoat™ is prepared by dissolving the ethylcellulose in a water immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudo-latex during the manufacturing phase. Thus, prior to using the same as a coating, it is necessary to intimately mix the Aquacoat™ with a suitable plasticizer prior to use.

Another aqueous dispersion of ethylcellulose is commercially available as Surelease™ (Colorcon, Inc., West Point, Pa., U.S.A.). This product is prepared by incorporating plasticizer into the dispersion during the manufacturing process. A hot melt of a polymer containing for example a plasticizer such as dibutyl sebacate, and a stabilizer such as oleic acid is prepared as a homogeneous mixture, which is then diluted with an alkaline solution to obtain an aqueous dispersion which can be applied directly onto substrates.

### Acrylic Polymers

The hydrophobic material comprising the controlled release coating is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers. The acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral methacrylic esters.

Certain methacrylic acid ester type polymers are useful for preparing pH dependent coatings that may be used in accordance with the present invention.

For example, there are a family of copolymers synthesized from diethylaminoethyl methacrylate and other neutral methacrylic esters, also known as methacrylic acid copolymer or polymeric methacrylates, commercially available as Eudragit™ from Rohm Tech, Inc. There are several different types of Eudragit™. For example Eudragit™ E is an example of a methacrylic acid copolymer that swells and dissolves in acidic media. Eudragit™ L is a methacrylic acid copolymer which does not swell at about pH <5.7 and is soluble at about pH >6. Eudragit™ S does not swell at about pH<6.5 and is soluble at about pH >7. Eudragit™ L and Eudragit™ S are water swellable, and the amount of water absorbed by these polymers is pH dependent. However, dosage forms coated with Eudragit™ L and S are pH independent.

The acrylic coating comprises a mixture of two acrylic resin lacquers commercially available from Rohm Pharma under the Tradenames Eudragit™ L30D and Eudragit™ S30D, respectively. Eudragit™ L30D and Eudragit™ S30D are copolymers of acrylic and methacrylic esters with a low content of quaternary ammonium groups, the molar ratio of ammonium groups to the remaining neutral methacrylic esters being 1:20 in Eudragit™ L30D and 1:40 in Eudragit™ S30D. The mean molecular weight is about 150,000. The code designations RL (high permeability) and RS (low permeability) refer to the permeability properties of these agents. Eudragit™ RL/RS mixtures are insoluble in water and in digestive fluids. However, coatings formed from the same are swellable and permeable in aqueous solutions and digestive fluids.

The Eudragit™ RL/RS dispersions of the present invention may be mixed together in any desired ratio in order to ultimately obtain a sustained release formulation having a desirable dissolution profile. Desirable sustained release formulations may be obtained, for instance, from a retardant coating derived from 100% Eudragit™ RL, 50% Eudragit™ RL and 50% Eudragit™ RS, and 10% Eudragit™ RL Eudragit™ 90% RS. Of course, one skilled in the art will recognize that other acrylic polymers may also be used, such as, for example, Eudragit™ L.

### Plasticizers

In the present invention where the coating comprises an aqueous dispersion of a hydrophobic material, the inclusion of an effective amount of a plasticizer in the aqueous dispersion of hydrophobic material will further improve the physical properties of the sustained release coating. For example, because ethylcellulose has a relatively high glass transition temperature and does not form flexible films under normal coating conditions, it is preferable to incorporate a plasticizer into an ethylcellulose coating containing sustained release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers, such as acetylated monoglycerides, phthalate esters, castor oil, etc., may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate, tributyl citrate, dibutyl phthalate, and possibly 1,2-propylene glycol. Other plasticizers that have proved to be suitable for enhancing the elasticity of the films formed from acrylic films such as Eudragit™ RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

It has further been found that the addition of a small amount of talc reduces the tendency of the aqueous dispersion to stick during processing, and acts as a polishing agent.

### Processes for Preparing Coated Beads

When the aqueous dispersion of hydrophobic material is used to coat inert pharmaceutical beads such as nu-pariel 18/20 beads, a plurality of the resultant stabilized solid controlled release beads may thereafter be placed in a gelatin capsule in an amount sufficient to provide an effective controlled release dose when ingested and contacted by an environmental fluid, e.g., gastric fluid or dissolution media.

The stabilized controlled release bead formulations of the present invention slowly release the therapeutically active agent, e.g., when ingested and exposed to gastric fluids, and then to intestinal fluids. The controlled release profile of the formulations of the invention can be altered, for example, by varying the amount of overcoating with the aqueous dispersion of hydrophobic material, altering the manner in which the plasticizer is added to the aqueous dispersion of hydrophobic material, by varying the amount of plasticizer relative to hydrophobic material, by the inclusion of additional ingredients or excipients, by altering the method of manufacture, etc. The payload release profile of the product may also be modified by increasing or decreasing the thickness of the retardant coating.

Spheroids or beads coated with a therapeutically active agent are prepared, e.g., by dissolving the therapeutically active agent in water and then spraying the solution onto a substrate, for example, nu pariel 18/20 beads, using a Wuster insert. Optionally, additional ingredients are also added prior to coating the beads in order to assist the binding of the tramadol or its analog to the beads, and/or to color the solution, etc. For example, a product that includes hydroxypropylmethylcellulose, etc. with or without a colorant, such as Opadry™, commercially available from Colorcon, Inc., may be added to the solution and the solution mixed for about 1 hour prior to application of the same onto the beads. The resultant coated substrate, in this example beads, may then be optionally overcoated with a barrier agent, to separate the therapeutically active agent from the hydrophobic controlled release coating. An example of a suitable barrier agent is one that comprises hydroxypropylmethylcellulose. However, any film former known in the art may be used. It is preferred that the barrier agent does not affect the dissolution rate of the final product.

The beads may then be overcoated with an aqueous dispersion of the hydrophobic material. The aqueous dispersion of hydrophobic material preferably further includes an effective amount of plasticizer, e.g. triethyl citrate. Pre-formulated aqueous dispersions of ethylcellulose, such as Aquacoat™ or Surelease™, may be used. If Surelease™ is used, it is not necessary to separately add a plasticizer. Alternatively, pre-formulated aqueous dispersions of acrylic polymers such as Eudragit™ can be used. The coating solutions of the present invention preferably contain, in addition to the film former, plasticizer, and solvent system such as water and a colorant to provide elegance and product distinction. Color may be added to the solution of the therapeutically active agent instead, or in addition to the aqueous dispersion of hydrophobic material. For example, color be added to Aquacoat™ via the use of alcohol or propylene glycol based color dispersions, milled aluminum lakes and opacifiers such as titanium dioxide by adding color with shear to water soluble polymer solution and then using low shear to the plasticized Aquacoat™. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the release retarding effect of the coating.

The plasticized aqueous dispersion of hydrophobic material may be applied onto the substrate comprising the therapeutically active agent by spraying using any suitable spray equipment known in the art. In a preferred method, a Wurster fluidized bed system is used in which an air jet, injected from underneath, fluidizes the core material and effects drying while the acrylic polymer coating is sprayed on. A sufficient amount of the aqueous dispersion of hydrophobic material to obtain a predetermined controlled release of said therapeutically active agent when said coated substrate is exposed to aqueous solutions, such as gastric fluid, is preferably applied, taking into account the physical characteristics of the therapeutically active agent, the manner of incorporation of the plasticizer, etc. After coating with the hydrophobic material, a further overcoat of a film-former, such as Opadry™, is optionally applied to the beads. This overcoat is provided, if at all, in order to substantially reduce agglomeration of the beads.

The release of the therapeutically active agent from the controlled release formulation of the present invention can be further influenced and adjusted to a desired rate by the addition of one or more release modifying agents. Controlled release may be achieved in the alternative by providing one or more passageways through the coating through which the drug or a solution of the drug can diffuse. The ratio of hydrophobic material to water soluble material is determined by, among other factors, the release rate required to produce the desired therapeutic effect and the solubility characteristics of the materials selected.

The release modifying agents which function as pore formers may be organic or inorganic, and include materials that can be dissolved, extracted or leached from the coating in the environment of use. The pore-formers may comprise one or more hydrophilic materials such as hydroxypropylmethylcellulose.

The sustained release coatings of the present invention can also include erosion promoting agents such as starches and gums.

The sustained release coatings of the present invention can also include materials useful for making microporous lamina in the environment of use, such as polycarbonates comprised of linear polyesters of carbonic acid in which carbonate groups reoccur in the polymer chain. The release modifying agent may also comprise a semi-permeable polymer.

The release modifying agent can be preferably selected from hydroxypropylmethylcellulose, lactose, metal stearates, and mixtures of any of the foregoing.

The sustained release coatings of the present invention may also include an exit means comprising at least one passageway, orifice, or the like. The passageway may be formed by such methods as those disclosed in U.S. Pat. Nos. 3,845,770, 3,916,889, 4,063,064 and 4,088,864, all of which are hereby incorporated by reference. The passageway can have any shape such as round, triangular, square, elliptical, irregular, etc.

### Matrix Bead Formulations

The controlled release formulation is achieved via a matrix having a controlled release coating as set forth above. The present invention may also utilize a controlled release matrix that affords in vitro dissolution rates of the tramadol or its analog within the preferred ranges and that releases the tramadol or its analog in a pH dependent or pH independent manner. The materials suitable for inclusion in a controlled release matrix will depend on the method used to form the matrix.

For example, a matrix in addition to the tramadol or its analog and, optionally, a NMDA antagonist and an anticonvulsant and/or a tricyclic anti-depressant may include:

Hydrophilic and/or hydrophobic materials, such as gums, cellulose ethers, acrylic resins, protein derived materials; the list is not meant to be exclusive, and any pharmaceutically acceptable hydrophobic material or hydrophilic material which is capable of imparting controlled release of the active agent and which melts or softens to the extent necessary to be extruded may be used in accordance with the present invention.

Digestible, long chain (C₈ to C₅₀, especially C₁₂ to C₄₀), substituted or unsubstituted hydrocarbons, such as fatty acids, fatty alcohols, glyceryl esters of fatty acids, mineral and vegetable oils and waxes, and stearyl alcohol; and polyalkylene glycols.

Of these polymers, acrylic polymers, especially Eudragit™, RSPO, the cellulose ethers, especially hydroxyalkylcelluloses and carboxyalkylcelluloses, are preferred. The oral dosage form may contain between 1% and 80% by weight of at least one hydrophilic or hydrophobic material.

When the hydrophobic material is a hydrocarbon, the hydrocarbon preferably has a melting point of between 25 and 90 carbon atoms. Of the long chain hydrocarbon materials, fatty aliphatic alcohols are preferred. The oral dosage form may contain up to 60% (by weight) of at least one digestible, long chain hydrocarbon.

Preferably, the oral dosage form contains up to 60% by weight of at least one polyalkylene glycol.

The hydrophobic material is preferably selected from the group consisting of alkylcelluloses, acrylic and methacrylic acid polymers and copolymers, shellac, zein, hydrogenated castor oil, hydrogenated vegetable oil, or mixtures thereof. In certain preferred embodiments of the present invention, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cynaoethyl methacrylate, aminoalkyl methacrylate copolymer, polyacrylic acid, polymethacrylic acid, methacrylic acid alkylamine copolymer, polymethyl methacrylate, polymethacrylic acid anhydride, polymethacrylate, polyacrylamide, poly(methacrylic acid anhydride), and glycidyl methacrylate copolymers. In other embodiments, the hydrophobic material is selected from materials such as hydroxyalkylcellulo ses such as hydroxypropylmethylcellulose and mixtures of the foregoing.

Preferred hydrophobic materials are water-insoluble with more or less pronounced hydrophilic and/or hydrophobic trends. Preferably, the hydrophobic materials useful in the invention have a melting point from about 30 to about 200° C., preferably from about 45 to about 90° C. Specifically, the hydrophobic material may comprise natural or synthetic waxes, fatty alcohols such as lauryl, myristyl, stearyl, cetyl or preferably cetostearyl alcohol, fatty acids, including but not limited to fatty acid esters, fatty acid glycerides (mono-, di-, and tri-glycerides), hydrogenated fats, hydrocarbons, normal waxes, stearic aid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material that is normally solid at room temperature and has a melting point of from about 30 to about 100° C.

Suitable hydrophobic materials which may be used in accordance with the present invention include digestible, long chain (C₈ to C₅₀, especially C₁₂ to C₄₀), substituted or unsubstituted hydrocarbons, such as fatty acids, fatty alcohols, glyceryl esters of fatty acids, mineral and vegetable oils and natural and synthetic waxes. Hydrocarbons having a melting point of between 25 and 90° C. are preferred. Of the long chain hydrocarbon materials, fatty (aliphatic) alcohols are preferred in certain embodiments. The oral dosage form may contain up to 60% by weight of at least one digestible, long chain hydrocarbon.

Preferably, a combination of two or more hydrophobic materials is included in the matrix formulations. If an additional hydrophobic material is included, it is preferably selected from natural and synthetic waxes, fatty acids, fatty alcohols, and mixtures of the same. Examples include beeswax, carnauba wax, stearic acid and stearyl alcohol. This list is not meant to be exclusive.

One particular suitable matrix comprises at least one water soluble hydroxyalkyl cellulose, at least one C₁₂ to C₃₆, preferably C₁₄ to C₂₂, aliphatic alcohol and, optionally, at least one polyalkylene glycol. The at least one hydroxyalkyl cellulose is preferably a hydroxy (C₁ to C₆) alkyl cellulose, such as hydroxypropylcellulose, hydroxypropylmethylcellulose and, especially, hydroxyethylcellulose. The amount of the at least one hydroxyalkyl cellulose in the present oral dosage form will be determined, inter alia, by the precise rate of tramadol or its analog release required. The at least one aliphatic alcohol may be, for example, lauryl alcohol, myristyl alcohol or stearyl alcohol. In particularly preferred embodiments of the present oral dosage form, however, the at least one aliphatic alcohol is cetyl alcohol or cetostearyl alcohol. The amount of the at least one aliphatic alcohol in the present oral dosage form will be determined, as above, by the precise rate of tramadol or its analog release required. It will also depend on whether at least one polyalkylene glycol is present in or absent from the oral dosage form. In the absence of at least one polyalkylene glycol, the oral dosage form preferably contains between 20% and 50% by weight of the at least one aliphatic alcohol. When at least one polyalkylene glycol is present in the oral dosage form, then the combined weight of the at least one aliphatic alcohol and the at least one polyalkylene glycol preferably constitutes between 20% and 50% by weight of the total dosage.

The ratio of hydroxyalkyl cellulose or acrylic resin to the aliphatic alcohol/polyalkylene glycol determines, to a considerable extent, the release rate of the tramadol or its analog from the formulation. A ratio of the hydroxyalkyl cellulose to the aliphatic alcohol/polyalkylene glycol of between 1:2 and 1:4 is preferred, with a ratio of between 1:3 and 1:4 being particularly preferred.

The polyalkylene glycol may be, for example, polypropylene glycol or, which is preferred, polyethylene glycol. The number average molecular weight of the polyalkylene glycol is preferred between 1,000 and 15,000 especially between 1,500 and 12,000.

Another suitable controlled release matrix would comprise an alkylcellulose, especially ethyl cellulose, a C₁₂ to C₃₆ aliphatic alcohol and optionally a polyalkylene glycol.

The preferred matrix includes a pharmaceutically acceptable combination of at least two hydrophobic materials.

In addition to the above ingredients a controlled release matrix may also contain suitable quantities of other materials, for example diluents, lubricants, binders, granulating aids, colorants, flavorants and glidants that are conventionally used in the art of pharmaceutical formulation.

### Processes for Preparing Matrix Based Beads

In order to facilitate the preparation of a solid, controlled release, oral dosage form according to this invention, any method of preparing a matrix formulation known to those skilled in the art may be used. For example incorporation in the matrix may be effected, for example, by (a) forming granules comprising at least one water soluble hydroxyalkyl cellulose and tramadol or its analog or a tramadol or its analog salt; (b) mixing the hydroxyalkyl cellulose containing granules with at least one C₁₂ to C₃₆ aliphatic alcohol; and (c) optionally, compressing and shaping the granules. Preferably, the granules are formed by wet granulating the hydroxyalkyl cellulose/tramadol or its analog with water. In a particularly preferred embodiment of this process, the amount of water added during tie wet granulation step is preferably between 1.5 and 5 times, especially between 1.75 and 3.5 times, the dry weight of the tramadol or its analog.

A spheronizing agent, together with the active ingredient can be spheronized to form spheroids. Microcrystalline cellulose is preferred. A suitable microcrystalline cellulose is, for example, the material sold as Avicel PH 101™ (FMC Corporation). In such embodiments, in addition to the active ingredient and spheronizing agent, the spheroids may also contain a binder. Suitable binders, such as low viscosity water soluble polymers, will be well known to those skilled in the pharmaceutical arts. However water soluble hydroxy lower alkyl cellulose, such as hydroxypropylcellulose are preferred. Additionally, or alternatively, the spheroids may contain a water insoluble polymer, especially an acrylic polymer, an acrylic copolymer, such as a methacrylic acid-ethyl acrylate copolymer, or ethyl cellulose. In such embodiments, the sustained release coating will generally include a hydrophobic material such as (a) a wax, either alone or in admixture with a fatty alcohol, or (b) shellac or zein.

### Melt Extrusion Matrix

Sustained release matrices can also be prepared via melt-granulation or melt-extrusion techniques. Generally, melt-granulation techniques involve melting a normally solid hydrophobic material, such as a wax, and incorporating a powdered drug therein. To obtain a sustained release dosage form, it may be necessary to incorporate an additional hydrophobic substance, such as ethylcellulose or a water insoluble acrylic polymer, into the molten wax hydrophobic material. Examples of sustained release formulations prepared by melt granulation techniques as are found in U.S. Pat. No. 4,861,598, assigned to the Assignee of the present invention and hereby incorporated by reference in its entirety.

The additional hydrophobic material may comprise one or more water-insoluble wax like thermoplastic substances possibly mixed with one or more wax like thermoplastic substances being less hydrophobic than said one or more water insoluble wax like substances. In order to achieve constant release, the individual wax like substances in the formulation should be substantially non-degradable and insoluble in gastrointestinal fluids during the initial release phases. Useful water-insoluble wax like substances may be those with a water solubility that is lower than about 1:5,000 (w/w).

In addition to the above ingredients, a sustained release matrix may also contain suitable quantities of other materials, such as diluents, lubricants, binders, granulating aids, colorants, flavorants and glidants that are conventionally used in the pharmaceutical arts. The quantities of these additional materials will be sufficient to provide the desired effect to the desired formulation. In addition to the above ingredients, a sustained release matrix incorporating melt-extruded multiparticulates may also contain suitable quantities of other materials, such as diluents, lubricants, binders, granulating aids, colorants, flavorants and glidants that are conventional in the pharmaceutical art in amounts up to about 50% by weight of the particulate if desired.

Specific examples of pharmaceutically acceptable carriers and excipients that may be used to formulate oral dosage forms are described in the Handbook of Pharmaceutical Excipients, American Pharmaceutical Association (1986), incorporated by reference herein.

### Melt Extrusion Multiparticulates

The preparation of a suitable melt-extruded matrix according to the present invention may, for example, include the steps of blending tramadol or its analog, together with at least one hydrophobic material and preferably the additional hydrophobic material to obtain a homogeneous mixture. The homogeneous mixture is then heated to a temperature sufficient to at least soften the mixture sufficiently to extrude the same. The resulting homogeneous mixture is then extruded to form strands. The extrudate is preferably cooled and cut into multiparticulates by any means known in the art. The strands are cooled and cut into multiparticulates. The multiparticulates are then divided into unit doses. The extrudate preferably has a diameter of from about 0.1 to about 5 mm and provides sustained release of the therapeutically active agent for a time period of from about 8 to about 24 hours.

An optional process for preparing the melt extrusions of the present invention includes directly metering into an extruder a hydrophobic material, a therapeutically active agent, and an optional binder, heating the homogenous mixture; extruding the homogenous mixture to thereby form strands; cooling the strands containing the homogeneous mixture, cutting the strands into particles having a size from about 0.1 mm to about 12 mm, and dividing said particles into unit doses. In this aspect of the invention, a relatively continuous manufacturing procedure is realized.

The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

The melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a hydrophobic material as described herein. In this regard, the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

The oral dosage forms can be prepared to include an effective amount of melt-extruded multiparticulates within a capsule. For example, a plurality of the melt-extruded multiparticulates may be placed in a gelatin capsule in an amount sufficient to provide an effective sustained release dose when ingested and contacted by gastric fluid.

A suitable amount of the multiparticulate extrudate can be compressed into an oral tablet using conventional tableting equipment using standard techniques. Techniques and compositions for making tablets that are compressed and/or molded, capsules of hard and soft gelatin, and pills are also described in Remington's Pharmaceutical Sciences, (Arthur Osol, editor), 1553-1593 (1980), incorporated by reference herein.

The extrudate can be shaped into tablets as set forth in U.S. Pat. No. 4,957,681, (Klimesch, et al), described in additional detail above and hereby incorporated by reference.

Optionally, the sustained release melt-extruded multiparticulate systems or tablets can be coated, or the gelatin capsule can be further coated, with a sustained release coating such as the sustained release coatings described above. Such coatings preferably include a sufficient amount of hydrophobic material to obtain a weight gain level from about 2 to about 30 percent, although the overcoat may be greater depending upon the physical properties of the particular tramadol or its analog compound utilized and the desired release rate, among other things.

The melt extruded unit dosage forms of the present invention may further include combinations of melt extruded multiparticulates containing one or more of the therapeutically active agents disclosed above before being encapsulated. Furthermore, the unit dosage forms can also include an amount of an immediate release therapeutically active agent for prompt therapeutic effect. The immediate release therapeutically active agent may be incorporated as separate pellets within a gelatin capsule, or may be coated on the surface of the multiparticulates after preparation of the dosage forms such as within a controlled release coating or matrix base. The unit dosage forms of the present invention may also contain a combination of controlled release beads and matrix multiparticulates to achieve a desired effect.

The sustained release formulations of the present invention preferably slowly release the therapeutically active agent, such that when the dosage form is ingested and exposed to gastric fluids, and then to intestinal fluids a therapeutically desirable plasma level is obtained. The sustained release profile of the melt extruded formulations of the invention can be altered, for example, by varying the amount of retardant which may be a hydrophobic material, by varying the amount of plasticizer relative to hydrophobic material, by the inclusion of additional ingredients or excipients, or by altering the method of manufacture, etc.

The melt extruded material can be prepared without the inclusion of the therapeutically active agent, which is added thereafter to the extrudate. Such formulations typically will have the therapeutically active agent blended together with the extruded matrix material, and then the mixture would be tableted in order to provide a slow release formulation. Such formulations may be advantageous, for example, when the therapeutically active agent included in the formulation is sensitive to temperatures needed for softening the hydrophobic material and/or the retardant material.

The following examples illustrate various aspects of the present invention. They are not to be construed to limit the claims in any manner whatsoever.

### Example 1

### Capsule Formulation Containing Gabapentin

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION WITH GABAPENTIN

| | | Overage API % 5 | | | | |
|---|---|---|---|---|---|---|
| | NAME OF INGREDIENT | mg | mg | mg | mg | mg |
| | FORMULATION | I | II | III | IV | V |
| 1 | DXM•HCl•H2O | 42 | 42 | 54 | 36 | 54 |
| 2 | Tramadol•HCl | 39.9 | 39.9 | 39.9 | 57 | 39.9 |
| 3 | Gabapentin | 90 | 180 | 45 | 90 | 90 |
| 4 | MCC | 61.9 | 41.9 | 94.9 | 50.8 | 49.9 |
| 5 | SiO2 | 3.1 | 3.1 | 3.1 | 3.1 | 3.1 |
| 6 | SLS | 1.6 | 1.6 | 1.6 | 1.6 | 1.6 |
| 7 | MgStr | 1.6 | 1.6 | 1.6 | 1.6 | 1.6 |
| | Total | 240.1 | 310.1 | 240.1 | 240.1 | 240.1 |
| | Capsule Size | 2 | 1 | 2 | 2 | 2 |
| | Number of Capsules | 500 | 200 | 200 | 200 | 200 |

### Example 2

### Capsule Formulation Containing Prepabalin

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION WITH PREGABALIN

| | | Overage API % 5 | | | | |
|---|---|---|---|---|---|---|
| | NAME OF INGREDIENT | mg | mg | mg | mg | mg |
| | FORMULATION | I | II | III | IV | V |
| 1 | DXM•HCl•H2O | 42 | 42 | 54 | 36 | 54 |
| 2 | Tramadol•HCl | 39.9 | 39.9 | 39.9 | 57 | 39.9 |
| 3 | Pregabalin | 20 | 30 | 15 | 15 | 30 |
| 5 | MCC | 91.9 | 81.9 | 84.9 | 85.8 | 69.9 |
| 6 | SiO2 | 3.1 | 3.1 | 3.1 | 3.1 | 3.1 |
| 7 | SLS | 1.6 | 1.6 | 1.6 | 1.6 | 1.6 |
| 8 | MgStr | 1.6 | 1.6 | 1.6 | 1.6 | 1.6 |
| | Total | 200.1 | 200.1 | 200.1 | 200.1 | 200.1 |
| | Capsule Size | 2 | 2 | 2 | 2 | 2 |
| | Number of Capsules | 200 | 200 | 200 | 200 | 200 |

### Example 3

### Capsule Formulation Containing Amitriptyline or Milnacipran

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION WITH AMITRIPTYLINE
OR MILNACIPRAN

| | NAME OF | Overage % 5 | | | | | |
|---|---|---|---|---|---|---|---|
| | INGREDIENT | mg | mg | mg | mg | mg | mg |
| | FORMULATION | I | II | III | IV | V | VI |
| 1 | DXM•HCl•H2O | 42 | 54 | 36 | 42 | 54 | 36 |
| 2 | Tramadol•HCl | 39.9 | 39.9 | 57 | 39.9 | 39.9 | 57 |
| 3 | Milnacipran HCl | 11.5 | 5.8 | 11.5 | | | |
| 4 | Amitriptyline HCl | | | | 11.3 | 5.7 | 11.3 |
| 5 | MCC | 31.3 | 25 | 20 | 31.5 | 25 | 20.4 |
| 6 | SiO2 | 3.1 | 3.1 | 3.1 | 3.1 | 3.1 | 3.1 |
| 7 | SLS | 1.6 | 1.6 | 1.6 | 1.6 | 1.6 | 1.6 |
| 8 | MgStr | 1.6 | 1.6 | 1.6 | 1.6 | 1.6 | 1.6 |
| | Total | 131 | 131 | 130.8 | 131 | 130.9 | 131 |
| | Capsule Size | 3 | 3 | 3 | 3 | 3 | 3 |
| | Number of Capsules | 200 | 200 | 200 | 200 | 200 | 200 |

### Example 4

Capsule Formulation Containing Gabapentin and Milnacipran or Amitriptyline

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION WITH AMITRIPTYLINE
OR MILNACIPRAN

| | | Overage API % 5 | | | | | |
|---|---|---|---|---|---|---|---|
| | NAME OF | mg | mg | mg | mg | mg | mg |
| | INGREDIENT | I | II | III | IV | V | V |
| 1 | DXM•HCl•H2O | 42 | 54 | 36 | 42 | 54 | 36 |
| 2 | Tramadol•HCl | 39.9 | 39.9 | 57 | 39.9 | 39.9 | 57 |
| 3 | Gabapentin | 90 | 45 | 90 | 90 | 45 | 90 |
| 4 | Milnacipran HCl | 11.5 | 5.8 | 11.5 | | | |
| 5 | Amitriptyline HCl | | | | 11.3 | 5.7 | 11.3 |
| 6 | MCC | 31.3 | 70 | 20 | 31.5 | 70 | 20.4 |
| 7 | SiO2 | 3.1 | 3.1 | 3.1 | 3.1 | 3.1 | 3.1 |
| 8 | SLS | 1.6 | 1.6 | 1.6 | 1.6 | 1.6 | 1.6 |
| 9 | MgStr | 1.6 | 1.6 | 1.6 | 1.6 | 1.6 | 1.6 |
| | Total | 221 | 221 | 220.8 | 221 | 220.9 | 221 |
| | Capsule Size | 2 | 2 | 2 | 2 | 2 | 2 |
| | Number of Capsules | 200 | 200 | 200 | 200 | 200 | 200 |

### Example 5

### Capsule Formulation Containing Magnesium without Dextromethorphan

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION WITH GABAPENTIN + Mg

Overage
API % 5

| | NAME OF INGREDIENT | mg | mg |
|---|---|---|---|
| | FORMULATION | I | II |
| 1 | Magnesium Sulfate | 120 | 240 |
| 2 | Tramadol•HCl | 50 | 50 |
| 3 | Gabapentin | 100 | 100 |
| 5 | MCC | 31.9 | 1.9 |
| 6 | SiO2 | 3.1 | 3.1 |
| 7 | SLS | 1.6 | 1.6 |
| 8 | MgStr | 1.6 | 1.6 |
| | Total | 308.2 | 398.2 |
| | Capsule Size | 1 | 0 |
| | Number of Capsules | 500 | 300 |

### Example 6

### Capsule Formulation Containing Gabapentin/Pregabalin and Magnesium without Dextromethorphan

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION WITH GABAPENTIN or
PREGABALIN + Mg

| | | Overage API % 5 | | | |
|---|---|---|---|---|---|
| | NAME OF INGREDIENT | mg | mg | mg | mg |
| | FORMULATION | I | II | III | IV |
| 1 | Magnesium Sulfate | 120 | 240 | 120 | 240 |
| 2 | Tramadol•HCl | 50 | 50 | 50 | 50 |
| 3 | Gabapentin | 100 | 100 | | |
| 4 | Pregabalin | | | 25 | 25 |
| 5 | MCC | 31.9 | 1.9 | 21.9 | 11.9 |
| 6 | SiO2 | 3.1 | 3.1 | 3.1 | 3.1 |
| 7 | SLS | 1.6 | 1.6 | 1.6 | 1.6 |
| 8 | MgStr | 1.6 | 1.6 | 1.6 | 1.6 |
| | Total | 308.2 | 398.2 | 223.2 | 333.2 |
| | Capsule Size | 1 | 0 | 2 | 1 |
| | Number of Capsules | 300 | 200 | 200 | 200 |

### Example 7

### Capsule Formulation Containing Dextromethorphan & Mg

The following ingredients in each one of the capsule formulations were weighed accurately, ground using a pestle and mortar to fine and homogeneous powders. These powders were sieved through 100 mesh and filled into hard gelatin capsules. The composition of each capsule formulation is listed below.

CAPSULE FORMULATION WITH GABAPENTIN & Mg

| | | Overage APIs % 5 | | | |
|---|---|---|---|---|---|
| | NAME OF INGREDIENT | mg | mg | mg | mg |
| | FORMULATION | I | II | III | IV |
| 1 | DXM•HCl•H2O | 42 | 36 | 54 | 36 |
| 2 | Tramadol•HCl | 39.9 | 39.9 | 39.9 | 57 |
| 3 | Gabapentin | 90 | 100 | 45 | 90 |
| 4 | Magnesium Sulfate | 120 | 240 | 120 | 120 |
| 5 | MCC | 11.9 | 11.9 | 44.9 | 10.8 |
| 6 | SiO2 | 3.1 | 3.1 | 3.1 | 3.1 |
| 7 | SLS | 1.6 | 1.6 | 1.6 | 1.6 |
| 8 | MgStr | 1.6 | 1.6 | 1.6 | 1.6 |
| | Total | 310.1 | 434.1 | 310.1 | 320.1 |
| | Capsule Size | 1 | 0 | 1 | 1 |
| | Number of Capsules | 300 | 200 | 200 | 200 |

### Example 8

### Efficacy of the Combination Therapy in Humans

The capsules (formulation 1 described in Example 1) were provided to 6 females between ages 20 and 27 who have menstrual pain during periods. They were asked to take 1 capsule every 12 hours during the menstrual cycles. After 3 menstrual cycles, they reported the effect of the composition on their pain. According to 4 females, they required only 1 capsule to relieve the pain to the comfort level so that they can do their normal work and 2 females required 2 capsules per day to their full comfort level for normal work.

After the initial feed back from the 6 females, they were provided the capsules (formulation 1 described in Example 7) and were asked to follow the same protocols as in the previous study. After 3 menstrual cycles, they reported the same level of comfort for doing normal work and their pain was almost completely eliminated during the time of the therapy.

### LITERATURE CITED

The following citations are incorporated herein by reference:
1. Plesan A, Hedman U, Xu X J, Wiesenfeld-Hallin Z. Comparison of ketamine and dextromethorphan in potentiating the antinociceptive effect of morphine in rats. Anesth Analg 1998; 86: 825-9.
2. Klepstad P, Maurset A, Moberg E R, Oye I. Evidence of a role for NMDA receptors in pain perception. Eur J Pharmacol 1990; 187: 513-8.
3. Woolf C J, Chong M S. Preemptive analgesia-treating postoperative pain by preventing the establishment of central sensitization. Anesth Analg 1993; 77: 362-79.
4. Bem J L, Peck R. Dextromethorphan. An overview of safety issues. Drug Saf 1992; 7: 190-9.
5. Davies S N, Lodge D. Evidence for involvement of Nmethyl-D-aspartate receptors in ‘wind-up’ of class 2 neurones in the dorsal horn of the rat. Brain Res 1987; 424: 402-6.
6. Felsby S, Nielsen J, Arendt-Nielsen L, Jensen T S. NMDA receptor blockade in chronic neuropathic pain: a comparison of ketamine and magnesium chloride. Pain 1995; 64: 283-91.
7. Dickenson A H. A cure for wind up: NMDA receptor antagonists as potential analgesics. Trends Pharmacol Sci 1990; 11: 307-9.
8. Albers G W, Atkinson R P, Kelley R E, Rosenbaum D M. Safety, tolerability, and pharmacokinetics of the Nmethyl-D-aspartate antagonist dextrorphan in patients with acute stroke. Stroke 1995; 26: 254-8.
9. Benson W M, Stefko P L, Randall L O. Comparative pharmacology of levorphanol, racemorphan and dextrorphan and related methyl ethers. J Pharmacol Exp Ther 1953; 109: 189-200.
10. Karlsson M O, Dahlstro\ddot{m} N A, Neil A. Characterization of high-affinity binding sites for the antitussive [3H] noscapine in guinea pig brain tissue. Eur J Pharmacol 1988; 145: 195-203.
11. Albers G W, Sáenz R E, Moses J A Jr, Choi D W. Safety and tolerance of oral dextromethorphan in patients at risk from brain ischemia. Stroke 1991; 22: 1075-7.
12. Choi D W. Dextrorphan and dextromethorphan attenuate glutamate neurotoxicity. Brain Res 1987; 403: 333-6.
13. Dayer P, Desmeules J, Collart L. Pharmacology of tramadol Drugs 1997; 53 Suppl 2:18-24.
14. Lee C R, McTavish D, Sorkin E M. Tramadol. A preliminary review of its pharmacodynamic and pharmacokinetic properties, and therapeutic potential in acute and chronic pain states. Drugs 1993; 46(2):313-40.
15. Rodd E H. Chemistry of Carbon Compounds, Elsevier Publ, New York, 1960.
16. Testa B and Jenner P Inhibitors Of Cytochrome P-450s and Their Mechanism of Action, DRUG METABOLISM REVIEWS 1981; 12: 1-117.
17. Murray M and Reidy G F. Selectivity in the Inhibition of Mammalian Cytochrome P-450 By Chemical Agents, PHARMACOLOGICAL REVIEWS 1990; 42: 85-101.
18. Porter T D and Coon M J. Cytochrome P-450: Multiplicity of Isoforms, Substrates, and Catalytic and Regulatory Mechanisms, J BIOL CHEM 1991; 266: 13469-13472.
19. Guengerich F P. Characterization of Human Microsomal Cytochrome P-450 Enzymes, ANNU REV PHARMACOL TOXICOL 1989; 29: 241-264 (1989).
20. George et al, Brain Res 1988; 440:35-379.
21. Unlugenc H, Gunduz M, Ozalevli M, Akman H. A comparative study on the analgesic effect of tramadol, tramadol plus magnesium, and tramadol plus ketamine for postoperative pain management after major abdominal surgery. Acta anaesthesiologica Scandinavica 2002; 46:1025-30.
22. Bull S A, et al., Discontinuing or switching selective serotonin-reuptake inhibitors, Annals of Pharmacotherapy 2002; 36(4): 578-584.
23. Barbui C, et al., Selective serotonin reuptake inhibitors versus tricyclic and heterocyclic antidepressants: comparison of drug adherence., In: The Cochrane Library, Issue 4, 2000. Oxford: Update Software.
24. Skaehill P A and Welch E B, SSRI withdrawal syndrome, 1997 American Society of Consultant Pharmacists, Inc.
25. Lieberman J A, History of the use of antidepressants in primary care, Primary Care Companion, Journal of Clinical Psychiatry 2003; 5 (supplement 7).
26. Field M J, Hughes J, and Singh L (2000) Further evidence for the role of the alpha2delta subunit of voltage dependent calcium channels in models of neuropathic pain. Br J Pharmacol 131: 282-286.
27. Gee N S, Brown J P, Dissanayake V U K, Offord J, Thurlow R, and Woodruff G N (1996) The novel anticonvulsant drug, gabapentin (neurontin), binds to the alpha2delta subunit of a calcium channel. J Biol Chem 271: 5768-5776.
28. Gu Y and Huang L Y M (2002) Gabapentin potentiates N-methyl-D-aspartate receptor mediated currents in rat GABAergic dorsal horn neurons. Neurosci Lett 324: 177-180.
29. Lu Y and Westlund K N (1999) Gabapentin attenuates nociceptive behaviors in an acute arthritis model in rats. J Pharmacol Exp Ther 290: 214-219.
30. Taylor C P (2004) The biology and pharmacology of calcium channel alpha2-delta proteins. CNS Drug Rev 10: 183-188.
31. Taylor C P, Gee N S, Su T Z, Kocsis J D, Welty D F, Brown J P, Dooley D J, Boden P, and Singh L (1998) A summary of mechanistic hypotheses of gabapentin pharmacology. Epilepsy Res 29: 233-249.
32. Piechan J L, Donevan S D, Taylor C P, Dickerson M R, and Li Z (2004) Pregabalin, a novel anticonvulsant, analgesic and anxiolytic drug, exhibits class-specifric α2δ-1 and α2δ-2 calcium channel subunit binding. Soc Neurosci Abstr 30: 115.11.
33. Belliotti T, Ekhato I V, Capiris T, Kinsora J, Vartanian M G, Field M, Meltzer L T, Heffner T, Schwarz J B, Taylor C P, et al. (2005) Structure-activity relationships of pregabalin and analogs that target the α2δ protein. J Med Chem 48: 2294-2307.
34. Dooley D J, Donovan C M, Meder W P, and Whetzel S Z (2002) Preferential action of gabapentin and pregabalin at P/Q-type voltage-sensitive calcium channels: inhibition of K⁺-evoked [³H]-norepinephrine release from rat neocortical slices. Synapse 45: 171-190.
35. Dooley D J, Mieske C A, and Borosky S A (2000) Inhibition of K(+)-evoked glutamate release from rat neocortical and hippocampal slices by gabapentin. Neurosci Lett 280: 107-110.
36. Fehrenbacher J C, Taylor C P, and Vasko M R (2003) Pregabalin and gabapentin reduce release of substance P and CGRP from rat spinal tissues only after inflammation or activation of protein kinase C. Pain 105: 133-144.
37. Schreiber S, Backer M M, Pick C G. The antinociceptive effect of venlafaxine in mice is mediated through opioid and adrenergic mechanisms. Neuroscience Letters 1999; 273: 85-8.
38. Galer B S. Neuropathic pain of peripheral origin: advances in pharmacologic treatment. Neurology 1995; 45(suppl 9):S17-25.
39. McQuay H J, Tramèr M, Nye B A, Carroll D, Wiffen P J, Moore R A. A systematic review of antidepressants in neuropathic pain. Pain 1996; 68:217-27.
40. Sindrup S H, Jensen T S. Efficacy of pharmacological treatments of neuropathic pain: an update and effect related to mechanism of drug action. Pain 1999; 83:389-400.
41. Watson C P N, Vernich L, Chipman M, et al. Nortriptyline versus amitriptyline in postherpetic neuralgia. Neurology 1998; 51:1166-71.
42. Bryson H M, Wilde M I. Amitriptyline: a review of its pharmacological properties and therapeutic use in chronic pain states. Drugs Aging 1996; 8:459-76.
43. Magni G. The use of antidepressants in the treatment of chronic pain: a review of the current evidence. Drugs 1991; 42:730-48.
44. Garner E M, Kelly M W, Thompson D F. Tricyclic antidepressant withdrawal syndrome. Ann Pharmacother 1993; 27:1068-72.
45. Catterall W A (1995) Structure and function of voltage-gated ion channels. Annu Rev Biochem 64: 493-531.
46. Fozzard H A and Hanck D A (1996) Structure and function of voltage-dependent sodium channels: Comparison of brain II and cardiac isoforms. Physiol Rev 76: 887-926.
47. Song J H, Ham S S, Shin Y K, Lee C S. Amitriptyline modulation of Na channels in rat dorsal root ganglion neurons. Eur J Pharmacol 2000; 401: 297-305.
48. Sawynok J, Esser M J, Reid A R. Antidepressants as analgesics: an overview of central and peripheral mechanisms of action. J Psychiatry Neurosci 2001; 26: 21-8.
49. Puozzo C, Panconi E, Deprez D (2002). “Pharmacology and pharmacokinetics of milnacipran”. International clinical psychopharmacology 17 Suppl 1: S25-35.
50. Arnold L M, Goldenberg D L, Stanford S B, Lalonde J K, et al. (2007): Gabapentin in the treatment of fibromyalgia: A randomized, double-blind, placebo-controlled, multicenter trial. Arthritis Rheum., 56(4):1336-44.
51. Biasi G, Manca S, Manganelli S, Marcolongo R (1998): Tramadol in the fibromyalgia syndrome: a controlled clinical trial versus placebo. Int J Clin Pharmacol Res, 18(1):13-19.
52. Mark L P, Prost R W, Ulmer J L, et al. Pictorial review of glutamate excitotoxicity: fundamental concepts for neuroimaging. AJNR Am J Neuroradiol 2001; 22:1813-24.
53. Romano T J, Stiller Magnesium deficiency fibromyalgia syndrome. J Nutr Med 1994165-167.

We claim:

1. A method for the treatment of pain associated with dysmenorrhea by administering a pharmaceutical composition comprising a synergistic combination of:
a) between 30 mg and 45 mg of dextromethorphan (between 36 mg and 54 mg of dextromethorphan hydrochloride monohydrate);
b) between 35 mg and 50 mg of tramadol (between 39 mg and 57 mg of tramadol hydrochloride); and
c) between 45 mg and 180 mg of gabapentin;
wherein said dextromethorphan, said tramadol, and said gabapentin are in an immediate release formulation; wherein the said dextromethorphan potentiates the pain relieving effect of tramadol and the gabapentin; the said tramadol potentiates the pain relieving effect of dextromethorphan and the gabapentin; and the said gabapentin potentiates the pain relieving effect of dextromethorphan and the tramadol.

2. A method for the treatment of pain associated with dysmenorrhea by administering a pharmaceutical composition comprising a synergistic combination of:
a) between 30 mg and 45 mg of dextromethorphan (between 36 mg and 54 mg of dextromethorphan hydrochloride monohydrate);
b) between 35 mg and 50 mg of tramadol (between 39.8 mg and 57 mg of tramadol hydrochloride); and
c) between 15 mg and 30 mg of pregabalin;
wherein said dextromethorphan, said tramadol, and said gabapentin are in an immediate release formulation; wherein the said dextromethorphan potentiates the pain relieving effect of tramadol and the gabapentin; the said tramadol potentiates the pain relieving effect of dextromethorphan and the gabapentin; and the said gabapentin potentiates the pain relieving effect of dextromethorphan and the tramadol.

3. The method for the treatment of pain associated with dysmenorrhea according to claims 1 and 2, wherein the pharmaceutical composition further contains magnesium (between 120 mg and 240 mg of magnesium sulfate or an equivalent pharmaceutically acceptable salt thereof).

4. A method for the treatment of pain associated with dysmenorrhea by administering a pharmaceutical composition comprising a synergistic combination of:
a) between 24 mg and 48 mg of magnesium (between 120 mg and 240 mg of magnesium sulfate or an equivalent pharmaceutically acceptable salt thereof);
b) about 50 mg of tramadol (about 39.8 mg of tramadol hydrochloride); and
c) about 100 mg of gabapentin or about 25 mg of pregabalin;
wherein said magnesium, said tramadol, and said gabapentin or pregabalin are in an immediate release formulation; wherein the said magnesium potentiates the pain relieving effect of tramadol and the gabapentin or pregabalin; the said tramadol potentiates the pain relieving effect of magnesium and the gabapentin or pregabalin; and the said gabapentin or pregabalin potentiates the pain relieving effect of magnesium and the tramadol.

5. The method for the treatment of pain associated with dysmenorrhea by administering a pharmaceutical composition according to claims 1-3, wherein the pharmaceutical composition is formulated for oral administration, a solution, a suspension or elixir for oral administration, an injectable formulation, comprised in an implantable device, a topical preparation, comprised in a solid state or depot type transdermal delivery device, a suppository, a buccal tablet, or an inhalation formulation.

6. The method for the treatment of pain associated with dysmenorrhea by administering a pharmaceutical composition according to claim 4, wherein the pharmaceutical composition is formulated for oral administration as a tablet or encapsulated multiparticulate formulation.

7. The method for the treatment of pain associated with dysmenorrhea by administering a pharmaceutical composition according to claims 1-3, wherein the composition is free or essentially free of a NSAID or acetaminophen.

8. The method according to claims 1-3, wherein the pharmaceutical composition is administered as a single formulation.
